SEQUENCE LISTING (42 sequences)

SEQ ID NO: 1 - DNA, 639 bp, Artificial Sequence
  HDAg genotype 1 A DNA sequence
agccgcagcgaaagcaaaaaaaaccgcggcggccgcgaagaaattctggaacagtgggtgggcgcgcgcaaaaaactggaagaactggaacgcgatctgcgcaaaattaaaaaaaaaattaaaaaactggaagaagaaaacccgtggctgggcaacattaaaggcattctgggcaaaaaagatcgcgaaggcgaaggcgcgccgccggcgaaacgcgcgcgcgcggatcagatggaagtggatagcggcccgcgcaaacgcccgtttcgcggcgaatttaccgataaagaacgccgcgatcatcgccgccgcaaagcgctggaaaacaaacgcaaacagctgagcagcggcggcaaaagcctgagcaaagaagaagaagaagaactgcgcaaactgaccgaagaagatgaacgccgcgaacgccgcgtggcgggcccgcgcgtgggcggcgtgaacccgctggaaggcggcacccgcggcgcgccgggcggcggctttgtgccgagcatgcagggcgtgccggaaagcccgtttgcgcgcaccggcgaaggcctggatgtgcgcggcaaccagggctttccgtgggatattctgtttccggcggatccgccgtttagcccgcagagctgccgcccgcag

SEQ ID NO: 2 - DNA, 639 bp, Artificial Sequence
  HDAg genotype 1 B DNA sequence
agccgcagcgaaagcaaaaaaaaccgcggcggccgcgaagaagtgctggaacagtgggtgaacggccgcaaaaaactggaagaactggaacgcgaactgcgccgcgcgcgcaaaaaaattaaaaaactggaagatgataacccgtggctgggcaacgtgaaaggcattctgggcaaaaaagataaagatggcgaaggcgcgccgccggcgaaacgcgcgcgcaccgatcagatggaaattgatagcggcccgcgcaaacgcccgctgcgcggcggctttaccgatcgcgaacgccaggatcatcgccgccgcaaagcgctgaaaaacaaaaaaaaacagctgagcgcgggcggcaaaagcctgagcaaagaagaagaagaagaactgaaacgcctgacccgcgaagatgaagaacgcaaaaaagaagaacatggcccgagccgcctgggcgtgaacccgagcgaaggcggcccgcgcggcgcgccgggcggcggctttgtgccgagcatgcagggcattccggaaagccgctttacccgcaccggcgaaggcctggatgtgcgcggcagccgcggctttccgcaggatattctgtttccgagcgatccgccgtttagcccgcagagctgccgcccgcag

SEQ ID NO: 3 - DNA, 639 bp, Artificial Sequence
  HDAg genotype 2 A DNA sequence
agccagagcgaaacccgccgcggccgccgcggcacccgcgaagaaaccctggaaaaatggattaccgcgcgcaaaaaagcggaagaactggaaaaagatctgcgcaaaacccgcaaaaccattaaaaaactggaagaagaaaacccgtggctgggcaacattgtgggcattattcgcaaaggcaaagatggcgaaggcgcgccgccggcgaaacgcccgcgcaccgatcagatggaagtggatagcggcccgggcaaacgcccgcataaaagcggctttaccgataaagaacgcgaagatcatcgccgccgcaaagcgctggaaaacaaaaaaaaacagctgagcgcgggcggcaaaattctgagcaaagaagaagaagaagaactgcgccgcctgaccgatgaagatgaagaacgcaaacgccgcgtggcgggcccgcgcgtgggcgatgtgaacccgagccgcggcggcccgcgcggcgcgccgggcggcggctttgtgccgcagatggcgggcgtgccggaaagcccgtttagccgcaccggcgaaggcctggatattcgcggcacccagggctttccgtgggtgagcccgagcccgccgcagcagcgcctgccgctgctggaatgcaccccgcag

SEQ ID NO: 4 - DNA, 639 bp, Artificial Sequence
  HDAg genotype 2 B DNA sequence
agccagagcgaaagcaaaaaaaaccgccgcggcggccgcgaagatattctggaaaaatggattaccacccgccgcaaagcggaagaactggaaaaagatctgcgcaaagcgcgcaaaaccattaaaaaactggaagatgaaaacccgtggctgggcaacattattggcattattcgcaaaggcaaagatggcgaaggcgcgccgccggcgaaacgcccgcgcaccgatcagatggaaattgatagcggcaccggcaaacgcccgcataaaagcggctttaccgataaagaacgcgaagatcatcgccgccgcaaagcgctggaaaacaaaaaaaaacagctgagcagcggcggcaaaaacctgagccgcgaagaagaagaagaactgggccgcctgaccgtggaagatgaagaacgccgccgccgcgtggcgggcccgcgcaccggcgatgtgaacctgagcggcggcggcccgcgcggcgcgccgggcggcggctttgtgccgcgcatggaaggcgtgccggaaagcccgtttacccgcaccggcgaaggcctggatattcgcggcaaccagggctttccgtgggtgcgcccgagcccgccgcagcagcgcctgccgctgctggaatgcaccccgcag

SEQ ID NO: 5 - protein, 213 aa, Artificial Sequence
  HDAg genotype 1 A polypeptide sequence
SRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSCRPQ

SEQ ID NO: 6 - protein, 213 aa, Artificial Sequence
  HDAg genotype 1 B polypeptide sequence
SRSESKKNRGGREEVLEQWVNGRKKLEELERELRRARKKIKKLEDDNPWLGNVKGILGKKDKDGEGAPPAKRARTDQMEIDSGPRKRPLRGGFTDRERQDHRRRKALKNKKKQLSAGGKSLSKEEEEELKRLTREDEERKKEEHGPSRLGVNPSEGGPRGAPGGGFVPSMQGIPESRFTRTGEGLDVRGSRGFPQDILFPSDPPFSPQSCRPQ

SEQ ID NO: 7 - protein, 213 aa, Artificial Sequence
  HDAg genotype 2 A polypeptide sequence
SQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLECTPQ

SEQ ID NO: 8 - protein, 213 aa, Artificial Sequence
  HDAg genotype 2 B polypeptide sequence
SQSESKKNRRGGREDILEKWITTRRKAEELEKDLRKARKTIKKLEDENPWLGNIIGIIRKGKDGEGAPPAKRPRTDQMEIDSGTGKRPHKSGFTDKEREDHRRRKALENKKKQLSSGGKNLSREEEEELGRLTVEDEERRRRVAGPRTGDVNLSGGGPRGAPGGGFVPRMEGVPESPFTRTGEGLDIRGNQGFPWVRPSPPQQRLPLLECTPQ

SEQ ID NO: 9 - DNA, 141 bp, Artificial Sequence
  PreS1 A DNA sequence
ggcaccaacctgagcaccagcaacccgctgggcttttttccggatcatcagctggatccggcgtttcgcgcgaacagcgcgaacccggattgggattttaacccgaacaaagatacctggccggatgcgaacaaagtgggc

SEQ ID NO: 10 - DNA, 141 bp, Artificial Sequence
  PreS1 B DNA sequence
ggccagaacctgagcaccagcaacccgctgggcttttttccggatcatcagctggatccggcgtttcgcgcgaacaccgcgaacccggattgggattttaacccgaacaaagatacctggccggatgcgaacaaagtgggc

SEQ ID NO: 11 - protein, 47 aa, Artificial Sequence
  PreS1 A polypeptide sequence
GTNLSTSNPLGFFPDHQLDPAFRANSANPDWDFNPNKDTWPDANKVG

SEQ ID NO: 12 - protein, 47 aa, Artificial Sequence
  PreS1 B polypeptide sequence
GQNLSTSNPLGFFPDHQLDPAFRANTANPDWDFNPNKDTWPDANKVG

SEQ ID NO: 13 - DNA, 66 bp, Artificial Sequence
  P2A autocatalytic peptide cleavage site DNA sequence
ggaagcggagctactaacttcagcctgctgaagcaggctggagacgtggaggagaaccctggacct

SEQ ID NO: 14 - protein, 22 aa, Artificial Sequence
  P2A autocatalytic peptide cleavage site polypeptide sequence
GSGATNFSLLKQAGDVEENPGP

SEQ ID NO: 15 - DNA, 3198 bp, Artificial Sequence
  Delta-1 codon optimized DNA sequence
atggccagcagaagtgaatcaaaaaagaatcggggagggcgggaagaaatcctggaacagtgggtcggagcacggaagaaactggaagaactggagagggacctgcgcaagatcaagaagaagatcaagaagctggaggaggagaacccctggctgggcaatatcaagggcatcctgggcaagaaggatcgggagggagagggagcaccacctgcaaagagggccagagccgaccagatggaggtggatagcggaccaaggaagcgccctttcagaggagagtttaccgacaaggagcggagagatcacaggcgccggaaggccctggagaacaagaggaagcagctgagctccggcggcaagtccctgtctaaggaggaggaggaggagctgcgcaagctgacagaggaggacgagagaagggagaggagggtggcaggacctagggtgggaggcgtgaacccactggagggaggaaccagaggagcacctggaggaggattcgtgccatccatgcagggagtgcccgagtctccttttgcccggacaggcgagggcctggatgtgagaggcaatcagggcttcccctgggacatcctgtttcctgccgatccacccttctctcctcagagctgccggccacagagcagatccgagtctaagaagaacaggggaggaagagaggaggtgctggagcagtgggtgaatggccggaagaagctggaggagctggagcgggagctgagaagggccagaaagaagatcaagaagctggaagacgataatccttggctgggcaatgtgaaaggcatcctgggcaagaaggacaaggatggagagggagcacctccagcaaagagggcaagaaccgaccagatggagatcgattctggaccaaggaagcgccccctgagaggaggcttcacagaccgggagagacaggatcaccgccggagaaaggccctgaagaacaagaagaagcagctgtccgccggaggcaagagcctgtccaaagaagaggaagaggagctgaagaggctgacccgcgaggacgaggagaggaagaaggaggagcacggaccaagcaggctgggagtgaatccttccgagggaggacctaggggagcaccaggaggaggcttcgtgccatctatgcagggcatccccgagagccggtttaccagaacaggagagggcctggacgtgaggggctcccgcggctttcctcaggacatcctgttcccatctgatccccctttttccccccagtcttgtaggcctcagggcaccaacctgtctacaagcaatccactgggcttctttcccgaccaccagctggatcctgccttccgcgccaacagcgccaatcccgactgggacttcaacccaaataaggacacctggccagatgccaacaaggtcggcggccagaacctgtccacatctaatcctctgggcttctttccagaccaccagctggatccagccttccgggccaacacagctaaccctgactgggacttcaaccccaataaggatacttggcccgacgccaacaaggtcggcggaagcggagctactaacttcagcctgctgaagcaggctggagacgtggaggagaaccctggacctatgagccagtccgagacaaggaggggccggagaggaaccagggaggagacactggagaagtggatcacagcccgcaagaaggccgaggagctggagaaggacctgcggaagaccagaaagacaatcaagaagctggaagaagagaacccatggctgggcaatatcgtgggcatcatcagaaagggcaaggacggcgagggagcaccaccagcaaagaggcccaggactgatcagatggaagtcgatagcggaccaggcaagcggcctcacaagtccggcttcacagacaaggagagagaggaccataggcgccggaaggccctggaaaacaagaagaagcaattatccgccggcggcaagatcctgtccaaagaggaagaagaggagctgagaaggctgaccgacgaggatgaggagaggaaaagaagggtggcaggaccaagggtgggcgacgtgaatcccagcaggggaggaccaagaggcgcccctggcggcggcttcgtgccacagatggcaggagtgccagagagccccttttccaggacaggagagggcctggatatcagaggcacccagggctttccttgggtgtctccaagccctccacagcagcggctgccactgctggagtgcacccctcagtcccagtctgagagcaagaagaacagaaggggcggcagagaggacatcctggagaagtggatcaccacacgcagaaaagctgaagaactggaaaaggacctgaggaaggcccgcaaaacaatcaagaagctggaggatgaaaatccatggctgggaaacatcatcggcatcatcaggaagggcaaggacggggaaggcgcaccacctgcaaagcggcctagaacagatcagatggaaatcgattctggcaccggcaagaggccacacaagagcggcttcaccgacaaggagcgcgaggatcacagaaggcgcaaggccctggagaacaagaagaagcaattaagcagcggcggcaagaatctgtccagagaagaagaggaggagctgggccgcctgaccgtggaggacgaggagcggagaaggcgcgtggcaggaccacgcacaggcgatgtgaacctgtccggaggaggaccaaggggagcacctggaggcggcttcgtgcctagaatggagggagtgcctgagtcccccttcacccgcaccggagagggcctggacatcagaggcaatcagggattcccatgggtgaggcccagcccaccacagcagcgcctgccactgctggagtgtaccccccagggcacaaacctgtccacctctaatcccctgggcttctttcctgatcatcagctggacccagccttcagggccaactccgccaatccagattgggacttcaacccgaataaggatacttggccagatgcaaacaaggtcggaggacagaacctgagcacatccaaccctctgggcttctttcctgaccatcagctggatcccgcctttcgcgccaataccgccaaccctgattgggacttcaaccctaataaggatacttggcctgatgctaataaggtcgggtga

SEQ ID NO: 16 - DNA, 3198 bp, Artificial Sequence
  Delta-2 codon optimized DNA sequence
atggccggcactaacctgtctacatcaaaccctctgggatttttccccgatcatcagctggaccccgcatttcgcgctaactctgctaaccctgactgggatttcaaccctaataaggacacatggccagatgccaacaaggtcggcggccagaacctgtccacctctaatcccctgggcttctttcctgaccaccagctggatcctgccttcagggccaacaccgccaatcccgactgggacttcaacccaaataaggatacctggcctgacgctaacaaggtcggcagccggtccgagtctaagaagaataggggaggaagggaggagatcctggagcagtgggtgggcgccagaaagaagctggaggagctggagcgggacctgagaaagatcaagaagaagatcaagaagctggaggaggagaacccctggctgggcaatatcaagggcatcctgggcaagaaggatcgggagggagagggagcaccacctgcaaagagggccagagccgaccagatggaggtggattccggccctaggaagcgcccattcagaggcgagtttacagacaaggagcggagagatcacaggcgccggaaggccctggagaacaagaggaagcagctgagctccggcggcaagagcctgtccaaggaggaggaggaggagctgcgcaagctgaccgaggaggacgagagaagggagaggagggtggcaggacctagggtgggaggcgtgaacccactggagggaggaacaagaggagcacccggaggaggcttcgtgccttctatgcagggcgtgcctgagagcccatttgccaggaccggagagggcctggacgtgagaggcaatcagggcttcccatgggacatcctgtttcccgccgatccacccttcagcccacagtcctgcaggccccagtctcgcagcgagtccaagaagaacagaggcggaagggaggaggtgctggagcagtgggtgaatggcaggaagaagctggaagaactggagagggagctgagaagggcccgcaagaagatcaagaagctggaagacgataatccttggctgggcaatgtgaaaggcatcctgggcaagaaggacaaggatggagagggagcacctccagcaaagagggcaagaacagaccagatggagatcgattccggaccaaggaagcgccctctgaggggaggcttcaccgaccgggagagacaggatcaccgccggagaaaggccctgaagaacaagaagaagcagctgagcgccggcggcaagtctctgagtaaagaagaagaggaggagctgaagcggctgacaagagaggacgaggagaggaagaaggaggagcacggaccatccaggctgggagtgaatccttctgagggaggaccaaggggcgcccctggcggaggcttcgtgcctagcatgcagggcatcccagagtccaggtttaccaggacaggcgaaggcctggacgtgcggggctctagaggctttccccaggacatcctgttccctagcgatccccctttttctcctcagagctgtagaccacagggaagcggagctactaacttcagcctgctgaagcaggctggagacgtggaggagaaccctggacctatgggcaccaacctgtccacatctaaccctctgggcttctttccagatcatcagctggacccagccttcagggccaacagcgccaatccagactgggacttcaaccccaataaggacacatggcctgacgcaaacaaggtcggaggacagaacctgagcacctccaatccactgggcttctttcccgaccaccagctggatccagccttccgcgccaacactgctaaccctgattgggacttcaaccctaataaggatacatggcctgatgccaataaggtcggctctcagagcgagacaaggaggggccggagaggaaccagggaggagacactggagaagtggatcaccgcccgcaagaaggccgaggagctggagaaggacctgaggaagacccgcaagacaatcaagaagctggaagaagagaacccatggctgggcaatatcgtgggcatcatcagaaagggcaaggacggcgagggagcaccaccagcaaagaggccccgcacagatcagatggaagtggattccggacctggcaagcggccacacaagtctggcttcaccgacaaggagagagaggaccataggcgccggaaggccctggaaaacaagaagaagcaattatctgccggcggcaagatcctgagtaaagaagaggaagaggagctgagaaggctgaccgacgaggatgaggagaggaagcgccgggtggccggcccacgcgtgggcgacgtgaatccctccaggggaggaccaagaggagcacctggaggcggcttcgtgccccagatggccggcgtgcccgagtcccctttttctcggaccggcgagggcctggatatcagaggcacacagggctttccatgggtgtccccctctcctccacagcagaggctgccactgctggagtgcacaccccagagccagagcgaatctaagaagaacagaaggggaggccgcgaggacatcctggaaaaatggatcaccacacgcagaaaagctgaagaactggaaaaggacctgcggaaggccagaaagaccatcaagaagctggaggatgaaaatccatggctgggaaacatcatcggcatcatccggaagggcaaggacggggaaggcgcaccacctgcaaagcggcctagaaccgatcagatggaaatcgatagcggcacaggcaagaggccacacaagtccggcttcaccgataaagagcgcgaggatcacagaaggcgcaaggccctggagaacaagaagaagcaattaagcagcggcggcaagaatctgtccagagaagaggaggaagagctgggccgcctgacagtggaggacgaggagcggagaaggcgcgtggcaggacccagaaccggcgatgtgaacctgtccggaggaggacctaggggagcaccaggaggcggcttcgtgcctagaatggagggcgtgccagagtctccctttacccggacaggcgagggcctggacatcagaggcaatcagggctttccctgggtccgcccctccccccctcagcagagactgccactgctggaatgcacaccacagtga

SEQ ID NO: 17 - DNA, 3762 bp, Artificial Sequence
  Delta-3 codon optimized DNA sequence
atggccggcaccaatctgtctacctcaaatcccctgggcttcttccccgatcatcagctggaccctgccttccgagcaaattccgctaatcctgattgggatttcaacccaaataaggacacatggccagatgccaacaaggtcggcggccagaacctgtccacctctaatcctctgggcttctttccagaccaccagctggatcccgccttcagggccaacacagccaatcccgactgggacttcaaccctaataaggacacctggcctgacgccaacaaggtcggcagcaggtccgagtctaagaagaataggggaggaagggaggagatcctggagcagtgggtgggagcacgcaagaagctggaggagctggagcgggacctgagaaagatcaagaagaagatcaagaagctggaggaggagaacccctggctgggcaatatcaagggcatcctgggcaagaaggatcgggagggagagggagcaccacctgcaaagagggccagagccgaccagatggaggtggattccggaccaaggaagcgccctttcagaggagagtttacagacaaggagcggagagatcacaggcgccggaaggccctggagaacaagcggaagcagctgagctccggcggcaagagcctgtccaaggaggaggaggaggagctgagaaagctgaccgaggaggacgagagaagggagaggagggtggccggccccagggtgggcggcgtgaaccctctggagggaggaacaaggggagcaccaggaggaggcttcgtgccttccatgcagggcgtgcccgagtctccttttgccaggaccggagagggcctggacgtgcgcggcaatcagggcttcccatgggacatcctgtttcccgccgatccacccttctctccccagagctgcaggcctcagtctcgcagcgagtccaagaagaacagaggcggaagggaggaggtgctggagcagtgggtgaatggcaggaagaagctggaagaactggagagggagctgagaagggcccgcaagaagatcaagaagctggaagacgataatccttggctgggcaatgtgaaaggcatcctgggcaagaaggacaaggatggagagggagcacctccagcaaagagggcaagaacagaccagatggagatcgattctggaccaaggaagcgccccctgaggggaggcttcaccgaccgggagagacaggatcaccgccggagaaaggccctgaagaacaagaagaagcagctgagcgccggcggcaagtctctgagtaaagaagaagaggaggagctgaagcggctgaccagagaggacgaggagcggaagaaggaggagcacggcccaagcagactgggagtgaatccatccgagggaggacctagaggcgcccctggcggcggcttcgtgccttctatgcagggcatcccagagagcaggtttaccaggacaggcgaaggcctggacgtgcggggctccagaggctttccccaggacatcctgttcccttctgatccccctttttccccacagtcttgtaggccccagggcaccaacctgtccacatctaacccactgggcttctttcctgatcaccagctggatccagccttccgcgccaactccgccaatccagactgggacttcaaccccaataaggacacatggcctgatgctaacaaggtcggaggccagaacctgagcacctccaatcccctgggcttctttcctgaccaccagctggatcctgccttccgcgccaacacagctaaccctgattgggacttcaacccaaataaggatacctggcctgatgcaaacaaggtcggaggaagcggagctactaacttcagcctgctgaagcaggctggagacgtggaggagaaccctggacctatgggcaccaacctgtctacaagcaatccactgggcttctttcccgaccatcagctggacccagccttcagggccaacagcgccaaccctgactgggacttcaacccaaataaggacacgtggcctgatgccaacaaggtcggaggacaaaacctgtccacctctaaccccctgggcttctttcccgatcatcaattagacccagccttccgcgctaacactgctaaccctgactgggacttcaacccgaataaggatacttggcctgatgccaataaggtcggcagccagtccgagacaaggaggggccggagaggaaccagggaggagacactggagaagtggatcaccgccagaaagaaggccgaggagctggagaaggacctgaggaagacccgcaagacaatcaagaagctggaagaagagaacccttggctgggcaatatcgtgggcatcatcagaaagggcaaggacggcgagggagcaccaccagccaagaggccacgcacagatcagatggaagtggatagcggaccaggcaagaggcctcacaagtccggcttcaccgacaaggagagggaggaccataggcgccggaaggccctggaaaacaagaagaagcaattatccgccggcggcaagatcctgtctaaagaagaggaagaagagctgagaaggctgaccgacgaggatgaggagaggaagaggagggtggcaggacctagagtgggcgacgtgaatccatccaggggaggaccaagaggagcaccaggaggcggcttcgtgccacagatggcaggagtgccagagagccccttttccaggacaggagagggcctggatatcaggggaacccagggctttccttgggtgtctccaagccctccacagcagcggctgccactgctggagtgcacaccccagtcccagtctgagagcaagaagaacagaaggggcggcagagaggacatcctggaaaaatggatcaccacacgcagaaaagctgaagaactggaaaaggacctgcggaaggccagaaagaccatcaagaagctggaggatgaaaatccatggctgggaaatatcatcggcatcatccggaagggcaaggacggggaaggcgcaccacctgcaaagcggcccaggaccgatcagatggaaatcgattctggaaccggcaagcggcctcacaagagtggcttcaccgataaggagagagaggatcacagaaggcgcaaggccctggagaacaagaagaagcaattaagcagcggcggcaagaatctgtccagagaagaggaagaggagctgggcagactgacagtggaggacgaggagcggagaaggcgcgtggcaggaccaagaaccggcgatgtgaacctgtccggaggaggaccaaggggagcacctgggggaggcttcgtgccaaggatggagggagtgcctgagtcccccttcaccagaaccggcgaaggcctggacatcaggggcaatcagggattcccatgggtgcggccctccccaccccagcagagactgcctctgctggagtgtaccccacagggcactaacctgtccacctctaacccgttaggcttctttcctgaccatcaattagatcccgccttccgggccaacagcgccaatcctgattgggacttcaacccgaataaggacacctggcccgacgcaaacaaggtcggagggcaaaacctgagcacctccaaccctttaggcttctttccagatcatcagctggatccagcctttagagccaataccgccaaccctgactgggatttcaaccctaacaaagatacctggcccgacgctaacaaagtgggatga

SEQ ID NO: 18 - DNA, 3900 bp, Artificial Sequence
  Delta-4 codon optimized DNA sequence
atggccagtcggagcgaatcaaagaaaaataggggagggcgggaagaaatcctggagcagtgggtcggagcacgaaagaaactggaagaactggagagggacctgcgcaagatcaagaagaagatcaagaagctggaggaggagaacccctggctgggcaatatcaagggcatcctgggcaagaaggatcgggagggagagggagcaccacctgcaaagagggccagagccgaccagatggaggtggatagcggccctaggaagcgcccattcagaggcgagtttacagacaaggagcggagagatcacaggcgccggaaggccctggagaacaagcggaagcagctgagctccggcggcaagtccctgtctaaggaggaggaggaggagctgagaaagctgaccgaggaggacgagagaagggagaggagggtggcaggacctagggtgggaggcgtgaacccactggagggaggaacaaggggagcacctggaggaggattcgtgccatccatgcagggagtgcctgagtctccatttgccaggaccggagagggcctggatgtgcgcggaaatcagggcttcccctgggacatcctgtttcctgccgatccacccttctccccacagtcttgcaggccacagggaaccaacctgagcacatccaatcctctgggcttctttccagaccaccagctggatcctgccttcagagccaactccgccaatccagactgggacttcaaccccaataaggacacatggcctgatgccaacaaggtcggcggccagaacctgtctaccagcaatcccctgggcttctttcctgaccaccagctggatccagccttccgggccaacactgctaaccctgattgggacttcaaccctaataaggatacctggccagacgccaacaaggtcggcggaagcggagctactaacttcagcctgctgaagcaggctggagacgtggaggagaaccctggacctatgtccaggtctgagagcaagaagaataggggaggaagagaggaggtgctggagcagtgggtgaacggccgcaagaagctggaggagctggagagggagctgagaagggcccgcaagaagatcaagaagctggaagacgataatccttggctgggcaatgtgaaaggcatcctgggcaagaaggacaaggatggagagggagcacctccagcaaagagggcaagaacagaccagatggagatcgattctggaccaaggaagcgccctctgaggggaggcttcaccgaccgggagagacaggatcaccgccggagaaaggccctgaagaacaagaagaagcagctgtccgccggcggcaagtccctgagcaaagaagaggaagaggagctgaagaggctgacccgcgaggacgaggagcggaagaaggaggagcacggaccaagcagactgggagtgaatccttccgagggaggaccaagaggagcacccggaggaggcttcgtgccatctatgcagggcatccccgagagccggtttaccagaacaggagagggcctggacgtgaggggctcccgcggctttcctcaggacatcctgttcccatctgatcccccttttagcccacagtcctgtaggccccagggcactaacctgagcacatccaacccactgggcttctttcctgatcatcagctggacccagccttccgcgccaacagcgccaaccctgactgggacttcaacccaaataaggacacatggccagatgctaacaaggtcggaggacaaaacctgtctaccagcaaccctctgggcttctttcccgatcatcagctggaccccgccttcagggccaacacagccaatcccgactgggacttcaacccgaataaggacacctggccagatgcaaacaaggtcggaggaagcggagctactaacttcagcctgctgaagcaggctggagacgtggaggagaaccctggacctatgagccagtctgagacaaggaggggccggagaggaaccagggaggagacactggagaagtggatcaccgccagaaagaaggccgaggagctggagaaggacctgcggaagaccagaaagacaatcaagaagctggaagaagagaacccatggctgggcaatatcgtgggcatcatccgcaagggcaaggacggcgagggagcaccaccagcaaagaggccccgcacagatcagatggaagtggatagcggccctggcaagaggccacacaagtccggcttcaccgacaaggagagggaggaccataggcgccggaaggccctggaaaacaagaagaagcaattatccgccggcggcaagatcctgtccaaagaggaagaagaggagctgagaaggctgaccgacgaggatgaggagaggaaaagaagggtggcaggaccaagagtgggcgacgtgaatcccagcagaggcggaccaagaggagcacctggaggcggcttcgtgccccagatggccggcgtgcccgagtctccttttagcagaactggagagggcctggatatcaggggaacacagggctttccatgggtgagcccatcccctccacagcagaggctgccactgctggagtgcacccctcagggaaccaacctgtctaccagcaacccgctgggcttctttcccgaccatcagctggaccctgccttccgcgccaactccgccaaccctgattgggacttcaacccgaataaggatacctggcccgacgctaacaaggtcggaggccagaacctgtccacctctaaccccttaggcttctttcccgatcaccagctggatcccgccttcagagccaacactgctaaccccgattgggacttcaacccgaataaggacacgtggccagacgctaacaaggtcgggggaagcggagctactaacttcagcctgctgaagcaggctggagacgtggaggagaaccctggacctatgtcgcagtccgagtctaagaagaatagaaggggcggccgggaggatatcctggaaaaatggatcaccacacgcagaaaagctgaagaactggaaaaggacctgaggaaggcccgcaagaccatcaagaagctggaggatgaaaatccatggctgggaaacatcatcggcatcatcagaaagggcaaggacggggaaggcgccccacctgcaaagcggcctagaaccgatcagatggaaatcgattctggcacaggcaagcggccacacaagagtggcttcaccgataaggagagagaggatcacagaaggcgcaaggccctggagaacaagaagaagcaattaagcagcggcggcaagaatctgtccagagaagaagaggaggagctgggcagactgacagtggaggacgaggagcggagaaggcgcgtggcaggaccaaggaccggcgatgtgaacctgagcggaggaggacctaggggagcaccaggaggcggcttcgtgcctaggatggagggagtgccagagtccccctttaccaggactggcgagggcctggacatcaggggaaatcagggattcccatgggtgcggcctagcccaccacagcagagactgccactgctggagtgtacaccccagggcacaaacctgagcacatccaatccgctgggcttctttccagatcatcaattagatccagccttcagggccaactccgccaatccggattgggacttcaacccgaataaggacacttggcccgacgcaaacaaggtcggagggcaaaacctgtctaccagcaatccacttggcttctttcctgaccatcagctggatcccgcctttcgcgccaataccgccaatcctgactgggacttcaatcctaacaaagacacctggcccgacgcaaacaaagtgggatga

SEQ ID NO: 19 - DNA, 2634 bp, Artificial Sequence
  Delta-5 codon optimized DNA sequence
atggcctcacggtcagagtcaaagaaaaataggggggggcgggaagaaatcctggaacagtgggtcggagcacggaaaaaactggaagagctggagagggacctgcgcaagatcaagaagaagatcaagaagctggaggaggagaacccctggctgggcaatatcaagggcatcctgggcaagaaggatcgggagggagagggagcaccacctgcaaagagggccagagccgaccagatggaggtggatagcggccctaggaagcgcccattcagaggcgagtttaccgacaaggagcggagagatcacaggcgccggaaggccctggagaacaagcggaagcagctgagctccggcggcaagtccctgtctaaggaggaggaggaggagctgagaaagctgacagaggaggacgagagaagggagcgccgggtggccggcccaagggtgggcggcgtgaaccccctggagggaggaaccaggggagcacctggaggaggcttcgtgccatctatgcagggcgtgcctgagagcccatttgccaggacaggagagggcctggatgtgcgcggcaatcagggcttcccctgggacatcctgtttcctgccgatccacccttcagcccacagtcctgcaggcctcagagcagatccgagtctaagaagaacaggggaggaagagaggaggtgctggagcagtgggtgaatggccggaagaagctggaggagctggagcgggagctgagaagggccagaaagaagatcaagaagctggaagacgataatccttggctgggcaatgtgaaaggcatcctgggcaagaaggacaaggatggagagggagcacctccagcaaagagggcaagaaccgaccagatggagatcgatagcggaccaaggaagcgccctctgagaggaggcttcacagaccgggagagacaggatcaccgccggagaaaggccctgaagaacaagaagaagcagctgtccgccggaggcaagagcctgtccaaagaagaggaagaggagctgaagaggctgacccgcgaggacgaggagcggaagaaggaggagcacggcccttccagactgggcgtgaatccatctgagggaggaccaaggggagcaccaggcggcggcttcgtgccaagcatgcagggcatccccgagtcccggtttaccagaacaggagagggcctggacgtgaggggctctcgcggctttcctcaggacatcctgttcccaagcgatccccctttttctccacagagctgtcgcccccagggaagcggagctactaacttcagcctgctgaagcaggctggagacgtggaggagaaccctggacctatgtctcagagcgagacaaggaggggccggagaggaaccagggaggagacactggagaagtggatcacagccagaaagaaggccgaggagctggagaaggacctgcggaagaccagaaagacaatcaagaagctggaagaagaaaatccatggctgggaaatatcgtgggcatcatcaggaagggcaaggacggcgagggagcaccaccagccaagaggcctcgcactgatcagatggaggtggattccggccctggcaagaggccacacaagtctggcttcacagacaaggagagggaggaccataggcgccggaaggccctggaaaacaagaagaagcaattatctgccggcggcaagatcctgagcaaagaggaagaggaggagctgagaaggctgaccgacgaggatgaggagaggaagaggagggtggcaggaccaagagtgggcgacgtgaatcctagcagaggcggaccaagaggcgccccaggcgggggcttcgtgccacagatggcaggagtgccagagtcccctttttctaggaccggagagggcctggatatcaggggaacacagggctttccatgggtgtccccatctcctccacagcagaggctgccactgctggagtgcacccctcagagccagtccgagtctaagaagaatagaaggggcggccgcgaggacatcctggagaagtggatcaccacacgcagaaaagctgaagaactggaaaaggacctgaggaaggcccgcaaaacaatcaagaagctggaggatgagaacccttggctgggcaatatcatcggaattatcaggaagggcaaggatggcgaaggcgccccacctgcaaagcggccaaggactgatcagatggaaatcgatagcggaacaggcaagcggccccacaagtccggcttcaccgacaaggagagagaggatcacagaaggcgcaaggccctggagaacaagaagaagcaattaagcagcggcggcaagaatctgtccagagaagaagaggaggagctgggcagactgaccgtggaggacgaggagcggagaaggcgcgtggcaggacctcgcacaggcgatgtgaacctgtccggaggaggacctaggggagcaccaggaggcggcttcgtgccacgcatggagggcgtgccagagtctccctttacccgcaccggagagggcctggacatcaggggcaatcagggctttccctgggtccgcccctccccccctcagcagagactgcccctgctggaatgcacaccacagtga

SEQ ID NO: 20 - DNA, 2772 bp, Artificial Sequence
  Delta-6 codon optimized DNA sequence
atggcctcacggtcagagtcaaagaagaacagaggcggaagagaagaaatcctggagcagtgggtcggagcacggaaaaagctggaagaactggagagggacctgcgcaagatcaagaagaagatcaagaagctggaggaggagaacccctggctgggcaatatcaagggcatcctgggcaagaaggatagggagggagagggagcaccacctgcaaagagggccagagccgaccagatggaggtggatagcggaccaaggaagcgccccttccgcggagagtttaccgacaaggagcggagagatcacaggcgccggaaggccctggagaacaagaggaagcagctgagctccggcggcaagtccctgtctaaggaggaggaggaggagctgcgcaagctgacagaggaggacgagagaagggagaggagggtggcaggaccaagggtgggaggagtgaatcctctggagggaggaaccagaggagcaccaggaggaggcttcgtgccaagcatgcagggagtgccagagtccccctttgccaggacaggagagggcctggacgtgagaggcaaccagggcttcccttgggacatcctgtttccagccgatccacccttcagccctcagtcctgcaggccacagggaagcggagctactaacttcagcctgctgaagcaggctggagacgtggaggagaaccctggacctatgagccggtccgagtctaagaagaataggggaggaagagaggaggtgctggagcagtgggtgaacggcagaaagaagctggaggagctggagagggagctgagaagggcccgcaagaagatcaagaagctggaagacgataatccttggctgggcaatgtgaaaggcatcctgggcaagaaggacaaggatggagagggagcacctccagcaaagagggcaagaaccgaccagatggagatcgatagcggacctaggaagcgcccactgaggggaggctttacagaccgggagagacaggatcaccgccggagaaaggccctgaagaacaagaagaagcagctgtccgccggaggcaagagcctgtccaaagaagaggaagaggagctgaagaggctgacccgcgaggacgaggagaggaagaaggaggagcacggaccatctaggctgggagtgaatcccagcgagggaggaccaaggggagcacctggaggaggcttcgtgccctccatgcagggcatccctgagtctcggtttaccagaaccggcgagggcctggacgtgaggggcagccgcggctttccacaggacatcctgttcccctccgatccccctttttctccccagagctgtcgccctcaaggaagcggagctactaacttcagcctgctgaagcaggctggagacgtggaggagaaccctggacctatgtctcagagcgagacaaggaggggccggagaggaaccagggaggagacactggagaagtggatcacagcccgcaagaaggccgaggagctggagaaggacctgcggaagaccagaaagacaatcaagaagctggaagaagagaacccttggctgggcaatatcgtgggcatcatcaggaagggcaaggacggcgagggagcaccaccagccaagaggccacgcactgatcagatggaggtggattctggaccaggcaagcggccccacaagagcggcttcacagacaaggagagagaggaccataggcgccggaaggccctggaaaacaagaagaagcaattaagcgccggcggcaagatcctgtccaaagaggaagaggaggagctgagaaggctgaccgacgaggatgaggagaggaaaagaagggtggcaggacctagggtgggcgacgtgaatccaagcaggggaggacctagaggagcaccaggaggcggcttcgtgccacagatggcaggagtgcctgagtccccattttctcggaccggcgagggcctggatatcagaggcacacagggcttcccctgggtgtccccttctcctccacagcagcggctgcctctgctggagtgcacccctcagggaagcggagctactaacttcagcctgctgaagcaggctggagacgtggaggagaaccctggacctatgtcgcagagcgaatctaagaagaatagaaggggcggcagagaggatatcctggagaagtggatcaccacacgcagaaaagctgaagaactggaaaaggacctgaggaaggcccgcaagaccatcaagaagctggaggatgaaaatccatggctgggaaatatcatcggcatcatccggaagggcaaggacggggaaggcgccccacctgcaaagcggcccaggactgatcagatggaaatcgattccggcacaggcaagaggcctcacaagtctggcttcacagataaagagcgcgaggatcacagaaggcgcaaggccctggagaacaagaagaagcaattatctagcggcggcaagaatctgtccagagaagaagaggaggagctgggccgcctgaccgtggaggacgaggagcggagaaggcgcgtggcaggaccaagaacaggcgatgtgaacctgtctggaggcggcccaaggggcgcccccggcggaggcttcgtgccaagaatggaaggcgtgccagagtccccttttacccggacaggggaaggcctggacattagaggcaatcagggctttccctgggtgcgaccaagcccccctcagcagcgactgcctctgctggagtgtacccctcagtga

SEQ ID NO: 21 - DNA, 1569 bp, Artificial Sequence
  Delta-7 codon optimized DNA sequence
atggcctcacggtctgagtcaaagaagaatcgggggggaagagaagaaatcctggaacagtgggtcggcgcacggaaaaaactggaagaactggagcgggacctgagaaagatcaagaagaagatcaagaagctggaggaagagaacccctggctgggcaatatcaagggcatcctgggcaagaaggatcgggagggcgagggagcaccacctgcaaagagggcaagggcagaccagatggaggtggattccggacctaggaagcggcccttccggggagagtttaccgacaaggagcggagagatcacaggcgccggaaggccctggagaacaagcggaagcagctgagctccggcggcaagtctctgagcaaggaggaggaggaggagctgagaaagctgacagaggaggacgagagaagggagcgccgggtggccggcccaagggtgggcggcgtgaaccccctggagggaggaaccaggggagcaccaggaggaggcttcgtgccttctatgcagggcgtgccagagagcccctttgccaggacaggagagggcctggatgtgcgcggcaatcagggcttcccatgggacatcctgtttcccgccgatccacccttctcccctcagtcttgcaggccacagtcccgctctgagagcaagaagaacaggggaggaagggaggaggtgctggagcagtgggtgaatggcaggaagaagctggaggagctggagcgggagctgagaagggccagaaagaagatcaagaagctggaagacgataatccttggctgggcaatgtgaaaggcatcctgggcaagaaggacaaggatggagagggagcacctccagcaaagagggcacgcaccgaccagatggagatcgattccggaccaaggaagcggcccctgaggggaggcttcacagacagggagcgccaggatcaccgccggagaaaggccctgaagaacaagaagaagcagctgtctgccggcggcaagtccctgtctaaagaagaggaggaggagctgaagcggctgaccagagaggacgaggagcggaagaaggaggagcacggcccttccagactgggcgtgaatccatctgagggaggaccaagaggcgcccctggcggaggcttcgtgcctagcatgcagggcatcccagagtccaggtttaccagaaccggagagggcctggacgtgcggggctctagaggctttccccaggacatcctgttccctagcgatcccccttttagcccccagtcctgtaggcctcagggcaccaacctgagcacatccaatccactgggcttctttccagaccaccagctggatccagccttccgcgccaacagcgccaatccagactgggacttcaaccccaataaggacacctggcctgatgccaacaaggtcggcggccagaacctgtctacaagcaatcctctgggcttctttcctgatcaccagctggatcctgcctttcgggccaatacagccaaccctgactgggacttcaatcctaacaaagacacttggcccgatgctaataaggtcggctga

SEQ ID NO: 22 - DNA, 1569 bp, Artificial Sequence
  Delta-8 codon optimized DNA sequence
atggccagtcagagcgagacccgcagaggacggagaggaacacgagaagagacactggagaaatggattacagcacggaagaaggcagaagagctggagaaggacctgaggaagacccgcaagacaatcaagaagctggaggaggagaacccctggctgggcaatatcgtgggcatcatcaggaagggcaaggatggagagggagcaccacctgccaagaggcctcgcacagaccagatggaggtggatagcggaccaggcaagcggcctcacaagtccggcttcaccgacaaggagagagaggatcaccggagaaggaaggccctggagaacaagaagaagcagctgtccgccggcggcaagatcctgtctaaggaggaggaggaggagctgcgccggctgacagacgaggatgaggagaggaagagaagggtggcaggaccaagggtgggcgacgtgaatccttctaggggaggaccaaggggagcaccaggaggaggcttcgtgcctcagatggccggcgtgccagagtctccctttagccggacaggcgagggcctggatatcagaggcacccagggctttccttgggtgtctccaagcccaccacagcagcggctgccactgctggagtgcacaccccagtcccagtctgagagcaagaagaacaggaggggaggaagagaggacatcctggagaagtggatcaccacaagaaggaaggccgaggagctggagaaggacctgcggaaggccagaaagaccatcaagaagctggaggatgaaaatccttggctgggaaatatcatcggaattattagaaaaggcaaggacggagagggagcacctccagcaaagcggccaagaacagaccagatggagatcgattctggaaccggcaagaggccccacaagagtggcttcaccgataaggagcgcgaggatcaccgccggagaaaggccctggaaaacaagaagaagcaattaagctccggcggcaagaatctgagcagagaagaagaggaggagctgggccgcctgacagtggaggacgaggagaggcgccggagagtggcaggacctagaaccggcgatgtgaacctgtccggaggcggcccaaggggagcacctggaggcggcttcgtgccacgcatggagggcgtgcctgagtctcccttcaccaggacaggagagggcctggacatcagaggcaatcagggattcccatgggtgcggcccagcccacctcagcagagactgcctctgctggagtgtaccccacagggcacaaacctgtccacctctaatcctctgggcttctttccagaccaccagctggatccagccttcagggccaactccgccaaccctgactgggacttcaaccctaataaggacacatggccagatgccaacaaggtcggcggccagaacctgagcacctccaatcccctgggcttctttcctgaccaccagctggatcccgcctttcgcgccaataccgccaatcccgactgggacttcaatccaaataaggacacctggcccgatgctaacaaagtgggatga

SEQ ID NO: 23 - DNA, 1287 bp, Artificial Sequence
  Delta-9 codon optimized DNA sequence
atggccagtcggagcgaatcaaagaaaaatagagggggaagagaagaaatcctggagcagtgggtcggggcacggaaaaaactggaagaactggagcgggacctgagaaagatcaagaagaagatcaagaagctggaggaagagaacccctggctgggcaatatcaagggcatcctgggcaagaaggatagggagggcgagggagcaccacctgcaaagagggcaagggcagaccagatggaggtggattccggaccaaggaagcggcccttccggggagagtttaccgacaaggagcggagagatcacaggcgccggaaggccctggagaacaagcggaagcagctgagctccggcggcaagtctctgagcaaggaggaggaggaggagctgagaaagctgacagaggaggacgagagaagggagaggagggtggcaggacctagggtgggaggcgtgaacccactggagggaggaaccaggggagcacctggaggaggctttgtgccatctatgcagggagtgccagagagccctttcgccaggacaggagagggcctggatgtgcgcggcaatcagggcttcccctgggacatcctgtttcctgccgatccacccttcagcccacagtcctgcaggccacagtcccgctctgagagcaagaagaacaggggaggaagggaggaggtgctggagcagtgggtgaatggccggaagaagctggaggagctggagcgggagctgagaagggccagaaagaagatcaagaagctggaagacgataatccttggctgggcaatgtgaaaggcatcctgggcaagaaggacaaggatggagagggagcacctccagcaaagagggcacgcaccgaccagatggagatcgattctggacctaggaagcggcccctgagaggaggctttacagacagggagcgccaggatcaccgccggagaaaggccctgaagaacaagaagaagcagctgagcgccggcggcaagtccctgtctaaagaagaggaggaggagctgaagcggctgaccagagaggacgaggagcggaagaaggaggagcacggaccatccagactgggagtgaatccttctgagggaggaccaagaggcgccccaggcggcggctttgtgccaagcatgcagggcatccccgagtccaggttcaccagaaccggcgaaggcctggatgtgcggggcagcagaggcttcccccaggatattctgtttccctccgacccccccttcagtccccagtcttgccgacctcagtga

SEQ ID NO: 24 - DNA, 1287 bp, Artificial Sequence
  Delta-10 codon optimized DNA sequence
atggcctcacagagcgaaacacggcgggggcggaggggaactagagaggaaacactggaaaaatggattacagcacggaaaaaggcagaggaactggagaaggacctgaggaagacccgcaagacaatcaagaagctggaggaggagaacccatggctgggcaatatcgtgggcatcatccggaagggcaaggatggagagggagcaccacctgcaaagaggccccgcaccgaccagatggaggtggattctggccctggcaagaggccacacaagagcggcttcacagacaaggagcgcgaggatcaccggagaaggaaggccctggagaacaagaagaagcagctgagcgccggcggcaagatcctgtccaaggaggaggaggaggagctgcgccggctgaccgacgaggatgaggagcggaagagaagggtggcaggaccaagagtgggcgacgtgaatccctctaggggaggaccaaggggagcacctggaggaggcttcgtgcctcagatggcaggagtgccagagtcccctttttctaggaccggagagggcctggatatcaggggaacacagggctttccatgggtgtctccaagcccaccacagcagaggctgccactgctggagtgcacccctcagtcccagtctgagagcaagaagaacaggaggggaggaagggaggacatcctggagaagtggatcaccacaagaaggaaggccgaggagctggagaaggacctgcggaaggccagaaaaacaatcaagaagctggaagatgagaacccctggctgggcaatatcatcggcatcatcagaaaaggcaaggacggcgagggagcacctccagcaaagcggcctagaaccgaccagatggagatcgattccggcacaggcaagcggccacacaagtctggcttcaccgacaaggagagagaggatcaccgccggagaaaggccctggaaaacaagaagaagcaattaagctccggcggcaagaatctgagcagagaagaagaggaggagctgggcagactgaccgtggaggacgaggagaggcgccggagagtggcaggacccagaacaggcgatgtgaacctgagcggaggaggacctaggggagcaccaggaggcggcttcgtgcctagaatggagggcgtgccagagtccccctttaccaggacaggagagggcctggacatcaggggcaatcagggctttccctgggtccgcccttcaccaccacagcagagactgcccctgctggaatgcacaccacagtga

SEQ ID NO: 25 - protein, 1065 aa, Artificial Sequence
  Delta-1 polypeptide sequence
MASRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSCRPQSRSESKKNRGGREEVLEQWVNGRKKLEELERELRRARKKIKKLEDDNPWLGNVKGILGKKDKDGEGAPPAKRARTDQMEIDSGPRKRPLRGGFTDRERQDHRRRKALKNKKKQLSAGGKSLSKEEEEELKRLTREDEERKKEEHGPSRLGVNPSEGGPRGAPGGGFVPSMQGIPESRFTRTGEGLDVRGSRGFPQDILFPSDPPFSPQSCRPQGTNLSTSNPLGFFPDHQLDPAFRANSANPDWDFNPNKDTWPDANKVGGQNLSTSNPLGFFPDHQLDPAFRANTANPDWDFNPNKDTWPDANKVGGSGATNFSLLKQAGDVEENPGPMSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLECTPQSQSESKKNRRGGREDILEKWITTRRKAEELEKDLRKARKTIKKLEDENPWLGNIIGIIRKGKDGEGAPPAKRPRTDQMEIDSGTGKRPHKSGFTDKEREDHRRRKALENKKKQLSSGGKNLSREEEEELGRLTVEDEERRRRVAGPRTGDVNLSGGGPRGAPGGGFVPRMEGVPESPFTRTGEGLDIRGNQGFPWVRPSPPQQRLPLLECTPQGTNLSTSNPLGFFPDHQLDPAFRANSANPDWDFNPNKDTWPDANKVGGQNLSTSNPLGFFPDHQLDPAFRANTANPDWDFNPNKDTWPDANKVG

SEQ ID NO: 26 - protein, 1065 aa, Artificial Sequence
  Delta-2 polypeptide sequence
MAGTNLSTSNPLGFFPDHQLDPAFRANSANPDWDFNPNKDTWPDANKVGGQNLSTSNPLGFFPDHQLDPAFRANTANPDWDFNPNKDTWPDANKVGSRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSCRPQSRSESKKNRGGREEVLEQWVNGRKKLEELERELRRARKKIKKLEDDNPWLGNVKGILGKKDKDGEGAPPAKRARTDQMEIDSGPRKRPLRGGFTDRERQDHRRRKALKNKKKQLSAGGKSLSKEEEEELKRLTREDEERKKEEHGPSRLGVNPSEGGPRGAPGGGFVPSMQGIPESRFTRTGEGLDVRGSRGFPQDILFPSDPPFSPQSCRPQGSGATNFSLLKQAGDVEENPGPMGTNLSTSNPLGFFPDHQLDPAFRANSANPDWDFNPNKDTWPDANKVGGQNLSTSNPLGFFPDHQLDPAFRANTANPDWDFNPNKDTWPDANKVGSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLECTPQSQSESKKNRRGGREDILEKWITTRRKAEELEKDLRKARKTIKKLEDENPWLGNIIGIIRKGKDGEGAPPAKRPRTDQMEIDSGTGKRPHKSGFTDKEREDHRRRKALENKKKQLSSGGKNLSREEEEELGRLTVEDEERRRRVAGPRTGDVNLSGGGPRGAPGGGFVPRMEGVPESPFTRTGEGLDIRGNQGFPWVRPSPPQQRLPLLECTPQ

SEQ ID NO: 27 - protein, 1253 aa, Artificial Sequence
  Delta-3 polypeptide sequence
MAGTNLSTSNPLGFFPDHQLDPAFRANSANPDWDFNPNKDTWPDANKVGGQNLSTSNPLGFFPDHQLDPAFRANTANPDWDFNPNKDTWPDANKVGSRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSCRPQSRSESKKNRGGREEVLEQWVNGRKKLEELERELRRARKKIKKLEDDNPWLGNVKGILGKKDKDGEGAPPAKRARTDQMEIDSGPRKRPLRGGFTDRERQDHRRRKALKNKKKQLSAGGKSLSKEEEEELKRLTREDEERKKEEHGPSRLGVNPSEGGPRGAPGGGFVPSMQGIPESRFTRTGEGLDVRGSRGFPQDILFPSDPPFSPQSCRPQGTNLSTSNPLGFFPDHQLDPAFRANSANPDWDFNPNKDTWPDANKVGGQNLSTSNPLGFFPDHQLDPAFRANTANPDWDFNPNKDTWPDANKVGGSGATNFSLLKQAGDVEENPGPMGTNLSTSNPLGFFPDHQLDPAFRANSANPDWDFNPNKDTWPDANKVGGQNLSTSNPLGFFPDHQLDPAFRANTANPDWDFNPNKDTWPDANKVGSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLECTPQSQSESKKNRRGGREDILEKWITTRRKAEELEKDLRKARKTIKKLEDENPWLGNIIGIIRKGKDGEGAPPAKRPRTDQMEIDSGTGKRPHKSGFTDKEREDHRRRKALENKKKQLSSGGKNLSREEEEELGRLTVEDEERRRRVAGPRTGDVNLSGGGPRGAPGGGFVPRMEGVPESPFTRTGEGLDIRGNQGFPWVRPSPPQQRLPLLECTPQGTNLSTSNPLGFFPDHQLDPAFRANSANPDWDFNPNKDTWPDANKVGGQNLSTSNPLGFFPDHQLDPAFRANTANPDWDFNPNKDTWPDANKVG

SEQ ID NO: 28 - protein, 1299 aa, Artificial Sequence
  Delta-4 polypeptide sequence
MASRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSCRPQGTNLSTSNPLGFFPDHQLDPAFRANSANPDWDFNPNKDTWPDANKVGGQNLSTSNPLGFFPDHQLDPAFRANTANPDWDFNPNKDTWPDANKVGGSGATNFSLLKQAGDVEENPGPMSRSESKKNRGGREEVLEQWVNGRKKLEELERELRRARKKIKKLEDDNPWLGNVKGILGKKDKDGEGAPPAKRARTDQMEIDSGPRKRPLRGGFTDRERQDHRRRKALKNKKKQLSAGGKSLSKEEEEELKRLTREDEERKKEEHGPSRLGVNPSEGGPRGAPGGGFVPSMQGIPESRFTRTGEGLDVRGSRGFPQDILFPSDPPFSPQSCRPQGTNLSTSNPLGFFPDHQLDPAFRANSANPDWDFNPNKDTWPDANKVGGQNLSTSNPLGFFPDHQLDPAFRANTANPDWDFNPNKDTWPDANKVGGSGATNFSLLKQAGDVEENPGPMSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLECTPQGTNLSTSNPLGFFPDHQLDPAFRANSANPDWDFNPNKDTWPDANKVGGQNLSTSNPLGFFPDHQLDPAFRANTANPDWDFNPNKDTWPDANKVGGSGATNFSLLKQAGDVEENPGPMSQSESKKNRRGGREDILEKWITTRRKAEELEKDLRKARKTIKKLEDENPWLGNIIGIIRKGKDGEGAPPAKRPRTDQMEIDSGTGKRPHKSGFTDKEREDHRRRKALENKKKQLSSGGKNLSREEEEELGRLTVEDEERRRRVAGPRTGDVNLSGGGPRGAPGGGFVPRMEGVPESPFTRTGEGLDIRGNQGFPWVRPSPPQQRLPLLECTPQGTNLSTSNPLGFFPDHQLDPAFRANSANPDWDFNPNKDTWPDANKVGGQNLSTSNPLGFFPDHQLDPAFRANTANPDWDFNPNKDTWPDANKVG

SEQ ID NO: 29 - protein, 877 aa, Artificial Sequence
  Delta-5 polypeptide sequence
MASRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSCRPQSRSESKKNRGGREEVLEQWVNGRKKLEELERELRRARKKIKKLEDDNPWLGNVKGILGKKDKDGEGAPPAKRARTDQMEIDSGPRKRPLRGGFTDRERQDHRRRKALKNKKKQLSAGGKSLSKEEEEELKRLTREDEERKKEEHGPSRLGVNPSEGGPRGAPGGGFVPSMQGIPESRFTRTGEGLDVRGSRGFPQDILFPSDPPFSPQSCRPQGSGATNFSLLKQAGDVEENPGPMSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLECTPQSQSESKKNRRGGREDILEKWITTRRKAEELEKDLRKARKTIKKLEDENPWLGNIIGIIRKGKDGEGAPPAKRPRTDQMEIDSGTGKRPHKSGFTDKEREDHRRRKALENKKKQLSSGGKNLSREEEEELGRLTVEDEERRRRVAGPRTGDVNLSGGGPRGAPGGGFVPRMEGVPESPFTRTGEGLDIRGNQGFPWVRPSPPQQRLPLLECTPQ

SEQ ID NO: 30 - protein, 923 aa, Artificial Sequence
  Delta-6 polypeptide sequence
MASRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSCRPQGSGATNFSLLKQAGDVEENPGPMSRSESKKNRGGREEVLEQWVNGRKKLEELERELRRARKKIKKLEDDNPWLGNVKGILGKKDKDGEGAPPAKRARTDQMEIDSGPRKRPLRGGFTDRERQDHRRRKALKNKKKQLSAGGKSLSKEEEEELKRLTREDEERKKEEHGPSRLGVNPSEGGPRGAPGGGFVPSMQGIPESRFTRTGEGLDVRGSRGFPQDILFPSDPPFSPQSCRPQGSGATNFSLLKQAGDVEENPGPMSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLECTPQGSGATNFSLLKQAGDVEENPGPMSQSESKKNRRGGREDILEKWITTRRKAEELEKDLRKARKTIKKLEDENPWLGNIIGIIRKGKDGEGAPPAKRPRTDQMEIDSGTGKRPHKSGFTDKEREDHRRRKALENKKKQLSSGGKNLSREEEEELGRLTVEDEERRRRVAGPRTGDVNLSGGGPRGAPGGGFVPRMEGVPESPFTRTGEGLDIRGNQGFPWVRPSPPQQRLPLLECTPQ

SEQ ID NO: 31 - protein, 522 aa, Artificial Sequence
  Delta-7 polypeptide sequence
MASRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSCRPQSRSESKKNRGGREEVLEQWVNGRKKLEELERELRRARKKIKKLEDDNPWLGNVKGILGKKDKDGEGAPPAKRARTDQMEIDSGPRKRPLRGGFTDRERQDHRRRKALKNKKKQLSAGGKSLSKEEEEELKRLTREDEERKKEEHGPSRLGVNPSEGGPRGAPGGGFVPSMQGIPESRFTRTGEGLDVRGSRGFPQDILFPSDPPFSPQSCRPQGTNLSTSNPLGFFPDHQLDPAFRANSANPDWDFNPNKDTWPDANKVGGQNLSTSNPLGFFPDHQLDPAFRANTANPDWDFNPNKDTWPDANKVG

SEQ ID NO: 32 - protein, 522 aa, Artificial Sequence
  Delta-8 polypeptide sequence
MASQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLECTPQSQSESKKNRRGGREDILEKWITTRRKAEELEKDLRKARKTIKKLEDENPWLGNIIGIIRKGKDGEGAPPAKRPRTDQMEIDSGTGKRPHKSGFTDKEREDHRRRKALENKKKQLSSGGKNLSREEEEELGRLTVEDEERRRRVAGPRTGDVNLSGGGPRGAPGGGFVPRMEGVPESPFTRTGEGLDIRGNQGFPWVRPSPPQQRLPLLECTPQGTNLSTSNPLGFFPDHQLDPAFRANSANPDWDFNPNKDTWPDANKVGGQNLSTSNPLGFFPDHQLDPAFRANTANPDWDFNPNKDTWPDANKVG

SEQ ID NO: 33 - protein, 428 aa, Artificial Sequence
  Delta-9 polypeptide sequence
MASRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSCRPQSRSESKKNRGGREEVLEQWVNGRKKLEELERELRRARKKIKKLEDDNPWLGNVKGILGKKDKDGEGAPPAKRARTDQMEIDSGPRKRPLRGGFTDRERQDHRRRKALKNKKKQLSAGGKSLSKEEEEELKRLTREDEERKKEEHGPSRLGVNPSEGGPRGAPGGGFVPSMQGIPESRFTRTGEGLDVRGSRGFPQDILFPSDPPFSPQSCRPQ

SEQ ID NO: 34 - protein, 428 aa, Artificial Sequence
  Delta-10 polypeptide sequence
MASQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLECTPQSQSESKKNRRGGREDILEKWITTRRKAEELEKDLRKARKTIKKLEDENPWLGNIIGIIRKGKDGEGAPPAKRPRTDQMEIDSGTGKRPHKSGFTDKEREDHRRRKALENKKKQLSSGGKNLSREEEEELGRLTVEDEERRRRVAGPRTGDVNLSGGGPRGAPGGGFVPRMEGVPESPFTRTGEGLDIRGNQGFPWVRPSPPQQRLPLLECTPQ

SEQ ID NO: 35 - DNA, 3434 bp, Artificial Sequence
  Wild-type Delta-7/Delta-8 fusion nucleic acid sequence
aagcttgcaccatggccggcaccaacctgagcaccagcaacccgctgggcttttttccggatcatcagctggatccggcgtttcgcgcgaacagcgcgaacccggattgggattttaacccgaacaaagatacctggccggatgcgaacaaagtgggcggccagaacctgagcaccagcaacccgctgggcttttttccggatcatcagctggatccggcgtttcgcgcgaacaccgcgaacccggattgggattttaacccgaacaaagatacctggccggatgcgaacaaagtgggcagccgcagcgaaagcaaaaaaaaccgcggcggccgcgaagaaattctggaacagtgggtgggcgcgcgcaaaaaactggaagaactggaacgcgatctgcgcaaaattaaaaaaaaaattaaaaaactggaagaagaaaacccgtggctgggcaacattaaaggcattctgggcaaaaaagatcgcgaaggcgaaggcgcgccgccggcgaaacgcgcgcgcgcggatcagatggaagtggatagcggcccgcgcaaacgcccgtttcgcggcgaatttaccgataaagaacgccgcgatcatcgccgccgcaaagcgctggaaaacaaacgcaaacagctgagcagcggcggcaaaagcctgagcaaagaagaagaagaagaactgcgcaaactgaccgaagaagatgaacgccgcgaacgccgcgtggcgggcccgcgcgtgggcggcgtgaacccgctggaaggcggcacccgcggcgcgccgggcggcggctttgtgccgagcatgcagggcgtgccggaaagcccgtttgcgcgcaccggcgaaggcctggatgtgcgcggcaaccagggctttccgtgggatattctgtttccggcggatccgccgtttagcccgcagagctgccgcccgcagagccgcagcgaaagcaaaaaaaaccgcggcggccgcgaagaagtgctggaacagtgggtgaacggccgcaaaaaactggaagaactggaacgcgaactgcgccgcgcgcgcaaaaaaattaaaaaactggaagatgataacccgtggctgggcaacgtgaaaggcattctgggcaaaaaagataaagatggcgaaggcgcgccgccggcgaaacgcgcgcgcaccgatcagatggaaattgatagcggcccgcgcaaacgcccgctgcgcggcggctttaccgatcgcgaacgccaggatcatcgccgccgcaaagcgctgaaaaacaaaaaaaaacagctgagcgcgggcggcaaaagcctgagcaaagaagaagaagaagaactgaaacgcctgacccgcgaagatgaagaacgcaaaaaagaagaacatggcccgagccgcctgggcgtgaacccgagcgaaggcggcccgcgcggcgcgccgggcggcggctttgtgccgagcatgcagggcattccggaaagccgctttacccgcaccggcgaaggcctggatgtgcgcggcagccgcggctttccgcaggatattctgtttccgagcgatccgccgtttagcccgcagagctgccgcccgcagggcaccaacctgagcaccagcaacccgctgggcttttttccggatcatcagctggatccggcgtttcgcgcgaacagcgcgaacccggattgggattttaacccgaacaaagatacctggccggatgcgaacaaagtgggcggccagaacctgagcaccagcaacccgctgggcttttttccggatcatcagctggatccggcgtttcgcgcgaacaccgcgaacccggattgggattttaacccgaacaaagatacctggccggatgcgaacaaagtgggcagccagagcgaaacccgccgcggccgccgcggcacccgcgaagaaaccctggaaaaatggattaccgcgcgcaaaaaagcggaagaactggaaaaagatctgcgcaaaacccgcaaaaccattaaaaaactggaagaagaaaacccgtggctgggcaacattgtgggcattattcgcaaaggcaaagatggcgaaggcgcgccgccggcgaaacgcccgcgcaccgatcagatggaagtggatagcggcccgggcaaacgcccgcataaaagcggctttaccgataaagaacgcgaagatcatcgccgccgcaaagcgctggaaaacaaaaaaaaacagctgagcgcgggcggcaaaattctgagcaaagaagaagaagaagaactgcgccgcctgaccgatgaagatgaagaacgcaaacgccgcgtggcgggcccgcgcgtgggcgatgtgaacccgagccgcggcggcccgcgcggcgcgccgggcggcggctttgtgccgcagatggcgggcgtgccggaaagcccgtttagccgcaccggcgaaggcctggatattcgcggcacccagggctttccgtgggtgagcccgagcccgccgcagcagcgcctgccgctgctggaatgcaccccgcagagccagagcgaaagcaaaaaaaaccgccgcggcggccgcgaagatattctggaaaaatggattaccacccgccgcaaagcggaagaactggaaaaagatctgcgcaaagcgcgcaaaaccattaaaaaactggaagatgaaaacccgtggctgggcaacattattggcattattcgcaaaggcaaagatggcgaaggcgcgccgccggcgaaacgcccgcgcaccgatcagatggaaattgatagcggcaccggcaaacgcccgcataaaagcggctttaccgataaagaacgcgaagatcatcgccgccgcaaagcgctggaaaacaaaaaaaaacagctgagcagcggcggcaaaaacctgagccgcgaagaagaagaagaactgggccgcctgaccgtggaagatgaagaacgccgccgccgcgtggcgggcccgcgcaccggcgatgtgaacctgagcggcggcggcccgcgcggcgcgccgggcggcggctttgtgccgcgcatggaaggcgtgccggaaagcccgtttacccgcaccggcgaaggcctggatattcgcggcaaccagggctttccgtgggtgcgcccgagcccgccgcagcagcgcctgccgctgctggaatgcaccccgcagggcaccaacctgagcaccagcaacccgctgggcttttttccggatcatcagctggatccggcgtttcgcgcgaacagcgcgaacccggattgggattttaacccgaacaaagatacctggccggatgcgaacaaagtgggcggccagaacctgagcaccagcaacccgctgggcttttttccggatcatcagctggatccggcgtttcgcgcgaacaccgcgaacccggattgggattttaacccgaacaaagatacctggccggatgcgaacaaagtgggctgatgagaattccgt

SEQ ID NO: 36 - DNA, 3431 bp, Artificial Sequence
  Codon optimized Delta-7/Delta-8 fusion nucleic acid sequence
aagcttgcaccatggcaggcaccaatctgtcaacctctaaccctctgggctttttccccgaccatcagctggaccccgcattccgagcaaactccgctaaccctgactgggatttcaacccaaataaggacacctggcccgatgccaataaggtcggcggccagaacctgtccacatctaatccactgggcttctttcccgaccaccagctggatcctgccttccgggccaacacagccaatcctgactgggacttcaaccctaataaggatacctggcccgatgccaacaaggtcggcagcaggtccgagtctaagaagaataggggaggaagggaggagatcctggagcagtgggtgggagcacgcaagaagctggaggagctggagcgggacctgagaaagatcaagaagaagatcaagaagctggaggaggagaacccctggctgggcaatatcaagggcatcctgggcaagaaggatcgggagggagagggagcaccacctgcaaagagggccagagccgaccagatggaggtggattccggccctaggaagcgcccattcagaggcgagtttaccgacaaggagcggagagatcacaggcgccggaaggccctggagaacaagcggaagcagctgagctccggcggcaagagcctgtccaaggaggaggaggaggagctgagaaagctgacagaggaggacgagagaagggagaggagggtggcaggacctagggtgggaggcgtgaacccactggagggaggaaccaggggagcacctggaggaggattcgtgccatccatgcagggagtgcctgagtctccatttgccaggacaggagagggcctggacgtgcgcggaaatcagggcttcccctgggacatcctgtttcctgccgatccacccttctcccctcagtcttgcaggccacagtctcgcagcgagtccaagaagaacagaggcggaagggaggaggtgctggagcagtgggtgaatggccggaagaagctggaagaactggagagggagctgagaagggcccgcaagaagatcaagaagctggaagacgataatccttggctgggcaatgtgaaaggcatcctgggcaagaaggacaaggatggagagggagcacctccagcaaagagggcaagaaccgaccagatggagatcgattctggaccaaggaagcgccctctgagaggaggcttcacagaccgggagagacaggatcaccgccggagaaaggccctgaagaacaagaagaagcagctgagcgccggcggcaagtctctgagtaaagaagaagaggaggagctgaagcggctgaccagagaggacgaggagcggaagaaggaggagcacggaccaagcagactgggagtgaatccttccgagggaggaccaagaggagcacccggaggaggcttcgtgccatctatgcagggcatccccgagagcaggtttaccagaaccggcgaaggcctggacgtgcggggctccagaggctttcctcaggacatcctgttcccatctgatcccccttttagcccccagtcctgtaggcctcagggcaccaacctgtccacatctaaccctctgggcttctttcctgatcatcagctggacccagccttccgcgccaacagcgccaatcccgattgggacttcaacccaaataaggatacctggccagacgctaacaaggtcggaggacagaacctgagcacatccaatcctctgggcttctttccagaccaccagctggatccagccttcagggctaatacagccaatcccgactgggacttcaacccaaataaggacacgtggccagacgcaaacaaggtcggctctcagagcgagacaaggaggggccggagaggaaccagggaggagacactggagaagtggatcaccgccagaaagaaggccgaggagctggagaaggacctgaggaagacccgcaagacaatcaagaagctggaagaagagaacccatggctgggcaatatcgtgggcatcatccgcaagggcaaggacggcgagggagcaccaccagcaaagaggcccaggactgatcagatggaggtggatagcggccctggcaagaggccacacaagtccggcttcacagacaaggagagggaggaccataggcgccggaaggccctggaaaacaagaagaagcaattatccgccggcggcaagatcctgtctaaagaagaggaagaagagctgagaaggctgaccgacgaggatgaggagaggaaaagaagggtggcaggaccaagagtgggcgacgtgaatccctccagaggcggaccaagaggagcacctggaggcggcttcgtgccccagatggccggcgtgcccgagtctccttttagcagaaccggagagggcctggatatcaggggaacccagggctttccatgggtgtccccatctcctccacagcagcggctgccactgctggagtgcacccctcagagccagagcgaatctaagaagaacagaaggggcggcagagaggacatcctggaaaaatggatcaccacacgcagaaaagctgaagaactggaaaaggacctgcggaaggccagaaaaacaatcaagaagctggaggatgaaaatccatggctgggaaacatcatcggcatcatcagaaagggcaaggacggggaaggcgcaccacctgcaaagcggcctagaacagatcagatggaaatcgattctggaaccggcaagcggccacacaagagcggcttcaccgacaaggagagagaggatcacagaaggcgcaaggccctggagaacaagaagaagcaattaagcagcggcggcaagaatctgtccagagaagaggaagaggagctgggcagactgaccgtggaggacgaggagcggagaaggcgcgtggcaggacccaggacaggcgatgtgaacctgagcggaggaggacctaggggagcaccaggaggcggcttcgtgcctaggatggagggagtgccagagtccccctttaccagaaccggcgagggcctggacatcaggggaaatcagggattcccatgggtgcggccttccccaccacagcagagactgccactgctggagtgtaccccccagggcacaaacctgagcacctccaatcccctgggcttctttcctgaccatcagctggaccctgccttcagggccaacagcgccaacccagattgggacttcaaccctaataaggacacctggccagatgcaaacaaggtcggcggccaaaacctgtctacaagcaaccccctgggcttctttccagatcaccagctggatcccgcctttcgcgccaataccgccaaccctgattgggatttcaaccctaacaaagacacttggcctgacgctaataaggtcggctgatgagaattc

SEQ ID NO: 37 - protein, 1136 aa, Artificial Sequence
  Delta-7/Delta-8 fusion polypeptide sequence
MAGTNLSTSNPLGFFPDHQLDPAFRANSANPDWDFNPNKDTWPDANKVGGQNLSTSNPLGFFPDHQLDPAFRANTANPDWDFNPNKDTWPDANKVGSRSESKKNRGGREEILEQWVGARKKLEELERDLRKIKKKIKKLEEENPWLGNIKGILGKKDREGEGAPPAKRARADQMEVDSGPRKRPFRGEFTDKERRDHRRRKALENKRKQLSSGGKSLSKEEEEELRKLTEEDERRERRVAGPRVGGVNPLEGGTRGAPGGGFVPSMQGVPESPFARTGEGLDVRGNQGFPWDILFPADPPFSPQSCRPQSRSESKKNRGGREEVLEQWVNGRKKLEELERELRRARKKIKKLEDDNPWLGNVKGILGKKDKDGEGAPPAKRARTDQMEIDSGPRKRPLRGGFTDRERQDHRRRKALKNKKKQLSAGGKSLSKEEEEELKRLTREDEERKKEEHGPSRLGVNPSEGGPRGAPGGGFVPSMQGIPESRFTRTGEGLDVRGSRGFPQDILFPSDPPFSPQSCRPQGTNLSTSNPLGFFPDHQLDPAFRANSANPDWDFNPNKDTWPDANKVGGQNLSTSNPLGFFPDHQLDPAFRANTANPDWDFNPNKDTWPDANKVGSQSETRRGRRGTREETLEKWITARKKAEELEKDLRKTRKTIKKLEEENPWLGNIVGIIRKGKDGEGAPPAKRPRTDQMEVDSGPGKRPHKSGFTDKEREDHRRRKALENKKKQLSAGGKILSKEEEEELRRLTDEDEERKRRVAGPRVGDVNPSRGGPRGAPGGGFVPQMAGVPESPFSRTGEGLDIRGTQGFPWVSPSPPQQRLPLLECTPQSQSESKKNRRGGREDILEKWITTRRKAEELEKDLRKARKTIKKLEDENPWLGNIIGIIRKGKDGEGAPPAKRPRTDQMEIDSGTGKRPHKSGFTDKEREDHRRRKALENKKKQLSSGGKNLSREEEEELGRLTVEDEERRRRVAGPRTGDVNLSGGGPRGAPGGGFVPRMEGVPESPFTRTGEGLDIRGNQGFPWVRPSPPQQRLPLLECTPQGTNLSTSNPLGFFPDHQLDPAFRANSANPDWDFNPNKDTWPDANKVGGQNLSTSNPLGFFPDHQLDPAFRANTANPDWDFNPNKDTWPDANKVG

SEQ ID NO: 38 - protein, 8 aa, Artificial Sequence
  Ovalbumin 257-264 CTL
SIINFEKL

SEQ ID NO: 39 - protein, 17 aa, Artificial Sequence
  Ovalbumin 323-339 Th
ISQAVHAAHAEINEAGR

SEQ ID NO: 40 - protein, 9 aa, Artificial Sequence
  HDAg peptide 1
KLEDDNPWL

SEQ ID NO: 41 - protein, 9 aa, Artificial Sequence
  HDAg peptide 2
KLEEENPWL

SEQ ID NO: 42 - protein, 9 aa, Artificial Sequence
  HDAg peptide 3
FPWDILFPA

(19) United States
(12) Patent Application Publication
(10) Pub. No.: US 2023/0079898 A1
(43) Pub. Date: Mar. 16, 2023

(54) COMPOSITIONS AND METHODS FOR TREATING AND PREVENTING HEPATITIS B AND D

(71) Applicant: Svenska Vaccinfabriken Produktion AB, Sollentuna (SE)

(72) Inventors: Matti Sällberg, Sollentuna (SE); Lars Frelin, Sollentuna (SE)

(21) Appl. No.: 17/794,786

(22) PCT Filed: Jan. 26, 2021
(86) PCT No.: PCT/US2021/015126

§ 371 Date: Jul. 22, 2022

Related U.S. Application Data
(60) Provisional application No. 62/966,970, filed on Jan. 28, 2020.

(51) Int. Cl. A61K 39/29; A61P 31/14; A61P 31/20

(52) U.S. Cl. CPC A61K 39/292; A61K 39/29; A61P 31/14; A61P 31/20

(57) ABSTRACT

Disclosed herein are immunogenic compositions or product combinations of engineered hepatitis B and hepatitis D nucleic acids, genes, peptides, or proteins that can be used to illicit an immune response against a hepatitis B and/or hepatitis D infection. Also disclosed are methods of using the immunogenic compositions or product combinations in subjects to generate immune responses against HBV and/or HDV by administering the compositions or combinations with a nucleic acid prime and polypeptide boost approach.

### CROSS-REFERENCE TO RELATED APPLICATIONS

This application claims the benefit of priority of U.S. Provisional Patent Application No. 62/966,970, filed Jan. 28, 2020, which is hereby expressly incorporated by reference in its entirety.

### REFERENCE TO SEQUENCE LISTING

The present application is being filed along with a Sequence Listing in electronic format. The Sequence Listing is provided as a file entitled SVF005SeqListing.TXT, which was created and last modified on Jan. 26, 2021, which is 141,377 bytes in size. The information in the electronic Sequence Listing is hereby expressly incorporated by reference in its entirety.

### FIELD

Aspects of the present disclosure relate generally to immunogenic compositions or product combinations of engineered hepatitis B (HBV) and hepatitis D (HDV) nucleic acids, genes, peptides, or proteins that can be used to illicit an immune response against an HBV and/or HDV infection. This immune response comprises, consists essentially of, or consists of activated immune cells that produce neutralizing antibodies and activated immune cells, such as T cells and B cells, against HBV and/or HDV. The disclosure also relates generally to methods of using the immunogenic compositions or product combinations in subjects to generate immune responses against HBV and/or HDV by administering the compositions or combinations with a homologous or heterologous nucleic acid and/or polypeptide prime and nucleic acid and/or polypeptide boost approach.

### BACKGROUND

Hepatitis is a disease resulting in swelling and inflammation of the liver. This disorder is commonly caused by viruses, five types of which are currently known (hepatitis A, B, C, D, and F). Hepatitis B infection can be either acute or chronic, with severe chronic infections causing chronic inflammation, fibrosis, cirrhosis, and hepatocellular carcinoma. The hepatitis B virus has a partially double-stranded circular DNA genome that enters the host nucleus and is transcribed by the host RNA polymerase into four viral mRNA molecules. These are used to translate viral proteins such as capsid proteins and surface antigens as well as produce more DNA genomes using a reverse transcriptase. Hepatitis D is a virusoid that relies on hepatitis B coinfection or superinfection to replicate. The circular single-stranded RNA of hepatitis D is amplified using host RNA polymerases, but also contains a single hepatitis D antigen (HDAg) gene. During hepatitis B and D coinfection or superinfection, intact hepatitis D viruses are packaged with an envelope containing hepatitis B surface antigens surrounding the RNA genome coated with HDAg protein. Incorporation of the hepatitis B surface antigens is essential for hepatitis D infectivity, as hepatitis D does not encode its own receptor binding proteins. Coinfection or superinfection with hepatitis D causes more severe complications, with increased risk of liver failure, cirrhosis, and cancer. There is a present need for effective immunogenic compositions and vaccines to establish prophylactic immunity against both hepatitis B and D infections.

### SUMMARY

The present disclosure relates generally to the use of recombinant nucleic acids, DNA, RNA, proteins, polypeptides, or peptides comprising REV and/or HDV antigens to induce immune responses, antibody production, immune protection, or immunity against HBV or MDV infections. In some embodiments, the recombinant nucleic acids, DNA, RNA, proteins, polypeptides, or peptides comprising HBV and/or MDV antigens are used in a DNA prime/protein boost composition approach. In some embodiments, this DNA prime/protein boost composition approach results in greater immune response, antibody production, immune protection, or immunity against HBV or HDV infections compared to DNA-only, protein-only, or organism-based immunogenic compositions.

Chronic hepatitis B and D virus (HBV/HDV) infections can cause cancer. Current HBV therapy using nucleoside analogues (NAS) is life-long and reduces but does not eliminate the risk of cancer. A hallmark of chronic hepatitis B is a dysfunctional HBV-specific T cell response. In some embodiments is an immunotherapy driven by naïve healthy T cells specific, for the HDV antigen (HDAg) to bypass the need for HBV-specific T cells in order to prime PreS1-specific T cells and PreS1 antibodies blocking HBV entry. In some embodiments, combinations of PreS1 and/or HDAg sequences were evaluated for induction of PreS1 antibodies and HBV- and HDV-specific T cells in vitro and in vivo. In some embodiments, neutralization of HBV by PreS1-specific murine and rabbit antibodies was evaluated in cell culture, and rabbit anti-PreS1 were tested for neutralization of HBV in mice repopulated with human hepatocytes. In some embodiments, adoptive transfer of PreS1 antibodies prevented or modulated HMI infection after a subsequent challenge in humanized mice.

In some embodiments, the nucleic acid or polypeptide compositions comprise sequences, genes, or polypeptides of HBV, HDV, PreS1, or HDAg. In some embodiments, the PreS1 is PreS1 A or PreS1 B. In some embodiments, the HDAg is HDAg genotype 1 strain A (1 A), HDAg genotype 1 strain B (1 B), HDAg genotype 2 strain A (2 A), or HDAg genotype 2 strain B (2 B). In some embodiments, the compositions also comprise an autocatalytic peptide cleavage site. In some embodiments, the autocatalytic peptide cleavage site is a P2A autocatalytic peptide cleavage site. In some embodiments, the PreS1 and HDAg components are grouped together in the compositions. In some embodiments, the PreS1 is downstream or immediately downstream of the HDAg sequence. In some embodiments, the PreS 1 and HDAg groups are separated by an autocatalytic peptide cleavage site. In some embodiments, the PreS1 and HDAg groups are separated by a P2A autocatalytic peptide cleavage site.

In some embodiments, the nucleic acid compositions are a plasmid, virus, bacteriophage, cosmid, fosmid, phagemid, bacterial artificial chromosome (BAC), yeast artificial chromosome (YAC), or human artificial chromosome (HAC). In some embodiments, the nucleic acid compositions are circular or linear. In some embodiments, the nucleic acid compositions are produced in a biological system, including but not limited to mammalian cells, human cells, bacteria cells, E. coli, yeast, S. cerevisiae, or other appropriate biological system. In some embodiments, the HBV and/or HDV nucleic acids or genes are found in a cassette that comprises elements needed to transcribe and translate the nucleic acids or genes in a biological system.

In some embodiments, the polypeptide compositions are properly folded or denatured. In some embodiments, the polypeptide compositions are produced in a biological system, including but not limited to mammalian, bacteria, yeast, insect, or cell-free recombinant expression systems. In some embodiments, the polypeptide compositions are produced in mammalian, human, primary, immortalized, cancer, stem, fibroblasts, human embryonic kidney (HEK) 293, Chinese Hamster Ovary (CHO), bacterial, Escherichia coli, yeast, Saccharomyces cerevisiae, Pichia pastoris, insect, Spodoptera frugiperda Sf9, or S. frugiperda Sf21 cells, or in a cell-free system. In some embodiments, the polypeptide compositions are purified using techniques known in the art, including but not limited to extraction, freeze-thawing, homogenization, permeabilization, centrifugation, density gradient centrifugation, ultracentrifugation, precipitation, SDS-PAGE, native PAGE, size exclusion chromatography, liquid chromatography, gas chromatography, hydrophobic interaction chromatography, ion exchange chromatography, anion exchange chromatography, cation exchange chromatography, affinity chromatography, immunoaffinity chromatography, metal binding chromatography, nickel column chromatography, epitope tag purification, or lyophilization.

In some embodiments, the nucleic acid or polypeptide compositions are administered to an animal, including but not limited to humans, mice, rats, rabbits, cats, dogs, horses, cows, pigs, sheep, monkeys, primates, or chickens. In some embodiments, the nucleic acid or polypeptide compositions are administered 1, 2, 3, 4, 5, 6, or 7 days, or 1, 2, 3, 4, 5, 6, 7, 8, 9, or 10 weeks, or 1, 2, 3, 4, 5, 6, 7, 8, 9, 10, 11, 12 months or any time within a range defined by any two of the aforementioned times between each dose. In some embodiments, the nucleic acid compositions are administered before the polypeptide compositions are administered. In some embodiments, the polypeptide compositions are administered before the nucleic acid compositions.

In some embodiments, the nucleic acid or polypeptide compositions are administered in an amount of 1, 10, 100, 1000 ng, or 1, 10, 100, 200, 300, 400, 500, 600, 700, 800, 900, 1000 μg, or 1, 10, 100, or 1000 mg or any amount within a range defined by any two of the aforementioned amounts. In some embodiments, the nucleic acid or polypeptide compositions are administered with excipients. In some embodiments, the nucleic acid or polypeptide compositions are administered with adjuvants. In some embodiments, the nucleic acid compositions are administered with in vivo electroporation.

In some embodiments, the immunogenicity of the nucleic acid or polypeptide compositions are assessed by measuring interferon gamma (IFNγ) producing immune cells using techniques known in the art, including ELISpot, measuring IgG antibody titer specific to HBV, HDV, HBV proteins, HBV nucleic acids, HDV proteins, HDV nucleic acids, PreS1, or HDAg, or measuring the neutralization activity of sera or purified antibodies from immunized animals in an in vitro or in vivo assay.

In some embodiments, the administration of the nucleic acid or polypeptide compositions provide transient, lasting, or permanent protection against an HBV or HDV infection. In some embodiments, the transient, lasting, or permanent protection against an HBV or HDV infection is superior to other immunogenic compositions. In some embodiments, the administration of the nucleic acid or polypeptide compositions is performed in conjunction with an antiviral therapy. In some embodiments, the administration of the nucleic acid or polypeptide compositions to provide transient, lasting, or permanent protection against an HBV or HDV infection is effective in humans. In some embodiments, the nucleic acid or polypeptide compositions are used as vaccines against HBV or HDV.

Preferred aspects of the present invention related to the following numbered alternatives:

1. An immunogenic composition or product combination comprising:

(a) a nucleic acid comprising at least one nucleic acid sequence encoding hepatitis D antigen (HDAg) and at least one nucleic acid sequence encoding PreS1; and

(b) a polypeptide comprising at least one HDAg polypeptide sequence and at least one PreS1 polypeptide sequence.

2. The immunogenic composition or product combination of alternative 1, wherein the at least one nucleic acid sequence encoding HDAg comprises SEQ ID NO: 1, SEQ ID NO: 2, SEQ ID NO: 3 or SEQ ID NO: 4, or any combination thereof.

3. The immunogenic composition or product combination of alternative 1 or 2, wherein the at least one nucleic acid sequence encoding PreS1 comprises SEQ ID NO: 9 or SEQ ID NO: 10 or both.

4. The immunogenic composition or product combination of any one of alternatives 1-3, wherein the nucleic acid is configured such that each HDAg nucleic acid sequence is grouped with a PreS1 nucleic acid sequence, and wherein the PreS1 nucleic acid sequence is immediately downstream of the HDAg nucleic acid sequence.

5. The immunogenic composition or product combination of alternative 4, further comprising at least one nucleic acid sequence encoding an autocatalytic peptide cleavage site, wherein the grouped HDAg and PreS1 nucleic acid sequences are separated by the at least one nucleic acid sequence encoding the autocatalytic peptide cleavage site.

6. The immunogenic composition or product combination of alternative 5, wherein the at least one nucleic acid sequence encoding the autocatalytic peptide cleavage site comprises a nucleic acid sequence selected from the group consisting of porcine teschovirus-1 2A (P2A), foot-and-mouth disease virus 2A (F2A), equine rhinitis A virus (ERAV) 2A (E2A) and Thosea asigna virus 2A (T2A) nucleic acid, and wherein each encoded autocatalytic peptide cleavage site may optionally include a GSG (glycine-serine-glycine) motif at its N-terminus.

7. The immunogenic composition or product combination of alternative 5 or 6, wherein the at least one nucleic acid sequence encoding the autocatalytic peptide cleavage site comprises SEQ ID NO: 13.

8. The immunogenic composition or product combination of any one of alternatives 1-7, wherein the nucleic acid is codon optimized for expression in a human.

9. The immunogenic composition or product combination of any one of alternatives 1-8, wherein the nucleic acid comprises a sequence having at least 80%, 85%, 90%, 95%, 99%, or 100% homology to SEQ ID NO: 15-24 or 35-36.

10. The immunogenic composition or product combination of any one of alternatives 1-9, wherein the nucleic acid comprises a sequence having at least 80%, 85%, 90%, 95%, 99%, or 100% homology to SEQ ID NO: 18, or SEQ ID NO: 35-36.

11. The immunogenic composition or product combination of any one of alternatives 1-10, wherein the at least one HD Ag polypeptide comprises SEQ ID NO: 5, SEQ ID NO: 6, SEQ ID NO: 7 or SEQ ID NO: 8 or any combination thereof.

12. The immunogenic composition or product combination of any one of alternatives 1-12, wherein the at least one PreS1 polypeptide sequence comprises SEQ ID NO: 11 or SEQ ID NO: 12 or both.

13. The immunogenic composition or product combination of any one of alternatives 1-12, wherein the at least one PreS1 polypeptide sequence is downstream of the at least one HDAg polypeptide sequence.

14. The immunogenic composition or product combination of any one of alternatives 1-13, wherein the polypeptide comprises a sequence having at least 80%, 85%, 90%, 95%, 99%, or 100% homology to the sequence of SEQ ID NO: 25-34 or 37.

15. The immunogenic composition or product combination of any of alternatives 1-14, wherein the polypeptide comprises a sequence having at least 80%, 85%, 90%, 95%, 99%, or 100% homology to the sequence of SEQ ID NO: 29, 31, 32, or 37.

16. The immunogenic composition or product combination of any one of alternatives 1-15, wherein the polypeptide is recombinantly expressed.

17. The immunogenic composition or product combination of alternative 16, wherein the polypeptide is recombinantly expressed in a mammalian, bacterial, yeast, insect, or cell-free system.

18. The immunogenic composition or product combination of any one of alternatives 1-17, further comprising an adjuvant.

19. The immunogenic composition or product combination of alternative 18, wherein the adjuvant is alum. QS-21 or MF59, or any combination thereof.

20. The immunogenic composition or product combination of any one of alternatives 1-19, wherein the nucleic acid comprises DNA.

21. The immunogenic composition or product combination of any one of alternatives 1-20, wherein the nucleic acid is provided in a recombinant vector.

22. A method of generating an immune response in a subject using the immunogenic composition or product combination set forth in any one of alternatives 1-21, comprising:

administering to the subject at least one prime dose comprising the nucleic acid; and

administering to the subject at least one boost dose comprising the polypeptide.

23. The method of alternative 22, wherein the at least one boost dose further comprises an adjuvant.

24. The method of alternative 23, wherein the adjuvant is alum, QS-21, or MF59, or any combination thereof.

25. The method of any one of alternatives 22-24, wherein the at least one boost dose is administered at least 1, 2, 3, 4, 5, 6, 7, 8, 9, 10, 11, 12, 24, 36, or 48 days or weeks after the at least one prime dose is administered or within a range of time defined by any two of the aforementioned time points e.g., within 1-48 days or 1-48 weeks.

26. The method of any one of alternatives 22-25, wherein the administration is provided enterally, orally, intranasally, parenterally, subcutaneously, intramuscularly, intradermally, or intravenously or any combination thereof.

27. The method of any one of alternatives 22-26, wherein the administration is performed in conjunction with an antiviral therapy.

28. The method of alternative 27, wherein the antiviral therapy comprises administration of entecavir, tenofovir, lamivudine, adefovir, telbivudine, emtricitabine, interferon-α, pegylated interferon-α, or interferon alfa-2b, or any combination thereof.

29. An immunogenic composition or product combination for use in the treatment of hepatitis B or hepatitis D, comprising:

(a) a nucleic acid comprising at least one nucleic acid sequence encoding hepatitis D antigen (HDAg) and at least one nucleic acid sequence encoding PreS1; and

(b) a polypeptide comprising at least one HDAg polypeptide sequence and at least one PreS1 polypeptide sequence.

30. The immunogenic composition or product combination for use in the treatment of hepatitis B or hepatitis D of alternative 29, wherein the at least one nucleic acid sequence encoding HDAg comprises SEQ ID NO: 1, SEQ ID NO: 2, SEQ ID NO: 3 or SEQ ID NO: 4, or any combination thereof.

31. The immunogenic composition or product combination for use in the treatment of hepatitis B or hepatitis D of alternative 29 or 30, wherein the at least one nucleic acid sequence encoding PreS1 comprises SEQ ID NO: 9 or SEQ ID NO: 10 or both.

32. The immunogenic composition or product combination for use in the treatment of hepatitis B or hepatitis D of any one of alternatives 29-31, wherein the nucleic acid is configured such that each HDAg nucleic acid sequence is grouped with a PreS1 nucleic acid sequence, and wherein the PreS1. nucleic acid sequence is immediately downstream of the HDAg nucleic acid sequence.

33. The immunogenic composition or product combination for use in the treatment of hepatitis B or hepatitis D of alternative 32, further comprising at least one nucleic acid sequence encoding an autocatalytic peptide cleavage site, wherein the grouped HDAg and PreS1 nucleic acid sequences are separated by the at least one nucleic acid sequence encoding the autocatalytic peptide cleavage site.

34. The immunogenic composition or product combination for use in the treatment of hepatitis B or hepatitis D of alternative 33, wherein the at least one nucleic acid sequence encoding the autocatalytic peptide cleavage site comprises a nucleic acid sequence selected from the group consisting of porcine teschovirus-1 2A (P2A), foot-and-mouth disease virus 2A (F2A), equine rhinitis A virus (ERAV) 2A (E2A) and Thosea asigna virus 2A (T2A) nucleic acid, and wherein each encoded autocatalytic peptide cleavage site may optionally include a GSG (glycine-serine-glycine) motif at its N-terminus.

35. The immunogenic composition or product combination for use in the treatment of hepatitis B or hepatitis D of alternative 33 or 34, wherein the at least one nucleic acid sequence encoding the autocatalytic peptide cleavage site comprises SEQ ID NO: 13.

36. The immunogenic composition or product combination for use in the treatment of hepatitis B or hepatitis D of any one of alternatives 29-35, wherein the nucleic acid is codon optimized for expression in a human.

37. The immunogenic composition or product combination for use in the treatment or inhibition of hepatitis B or hepatitis D of any one of alternatives 29-36, wherein the nucleic acid comprises a sequence having at least 80%, 85%, 90%, 95%, 99%, or 100% homology to SEQ ID NO: 15-24 or 35-36.

38. The immunogenic composition or product combination for use in the treatment of hepatitis B or hepatitis D of any one of alternatives 29-37, wherein the nucleic acid comprises SEQ ID NO: 18, or SEQ ID NO: 35-36.

39. The immunogenic composition or product combination for use in the treatment of hepatitis B or hepatitis D of any one of alternatives 29-38, wherein the at least one HDAg polypeptide comprises SEQ ID NO: 5, SEQ ID NO: 6, SEQ ID NO: 7 or SEQ ID NO: 8 or any combination thereof.

40. The immunogenic composition or product combination for use in the treatment of hepatitis B or hepatitis D of any one of alternatives 29-39, wherein the at least one PreS1 polypeptide sequence comprises SEQ ID NO: 11 or SEQ ID NO: 12 or both.

41. The immunogenic composition or product combination for use in the treatment of hepatitis B or hepatitis D of any one of alternatives 29-40, wherein the at least one PreS1 polypeptide sequence is downstream of the at least one HDAg polypeptide sequence.

42. The immunogenic composition or product combination for use in the treatment or inhibition of hepatitis B or hepatitis D of any one of alternatives 29-41, wherein the polypeptide comprises a sequence having at least 80%, 85%, 90%, 95%, 99%, or 100% homology to the sequence of SEQ ID NO: 25-34 or 37.

43. The immunogenic composition or product combination for use in the treatment of hepatitis B or hepatitis D of any of alternatives 29-42, wherein the polypeptide comprises a sequence having at least 80%, 85%, 90%, 95%, 99%, or 100% homology to the sequence of SEQ ID NO: 29, 31, 32, or 37.

44. The immunogenic composition or product combination for use in the treatment of hepatitis B or hepatitis D of any one of alternatives 29-43, wherein the polypeptide is recombinantly expressed.

45. The immunogenic composition or product combination for use in the treatment of hepatitis B or hepatitis D of alternative 44, wherein the polypeptide is recombinantly expressed in a mammalian, bacterial, yeast, insect, or cell-free system.

46. The immunogenic composition or product combination for use in the treatment of hepatitis B or hepatitis D of any one of alternatives 29-45, further comprising an adjuvant.

47. The immunogenic composition or product combination for use in the treatment of hepatitis B or hepatitis D of alternative 46, wherein the adjuvant is alum, QS-21 or MF59, or any combination thereof.

48. The immunogenic composition or product combination for use in the treatment of hepatitis B or hepatitis D of any one of alternatives 29-47, wherein the nucleic acid comprises DNA.

49. The immunogenic composition or product combination for use in the treatment of hepatitis B or hepatitis D of any one of alternatives 29-48, wherein the nucleic acid is provided in a recombinant vector.

### BRIEF DESCRIPTION OF THE DRAWINGS

In addition to the features described above, additional features and variations will be readily apparent from the following descriptions of the drawings and exemplary embodiments. It is to be understood that these drawings depict typical embodiments and are not intended to be limiting in scope.

FIGS. 1A-B depicts the nucleic acid or polypeptide constructs comprising HBV and/or HDV antigens used herein. Ten constructs are provided, Delta-1 (Δ-1, D1), Delta-2 (Δ-2, D2), Delta-3 (Δ-3, D3), Delta-4 (Δ-4, D4), Delta-5 (Δ-5, D5), Delta-6 (Δ-6, D6), Delta-7 (Δ-7, D7), Delta-8 (Δ-8, D8), Delta-9 (Δ-9, D9), and Delta-10 (Δ-10, D10) (FIG. 1A). Western blot confirms that the ten polypeptide constructs are properly expressed (FIG. 1B). GFP was used as a control for Western blot.

FIG. 2 depicts the constructs used for the DNA prime/protein boost composition approach. The DNA composition comprises the nucleic acid sequence of Δ-4, and the protein composition comprises the polypeptide sequence of either Δ-7 or Δ-8 or a fusion of Δ-7 and Δ-8.

FIGS. 3A-3E depict the quantification of interferon gamma (IFNγ) forming spots per 10⁶ cells on an ELISpot assay, which corresponds to T lymphocyte activation, of a purified white blood cell population from sera derived from mice immunized with HBV/HDV DNA compositions in response to exposure to various HBV or HDV antigens. Antigens include purified polypeptides comprising PreS1 A (SEQ ID NO: 11), PreS1 A (SEQ ID NO: 12), HDAg genotype 1 A (SEQ II) NO: 5, “HDAg gtp 1A-pool 1” and “HDAg gtp 1A-pool 2”), HDAg genotype 1 B (SEQ ID NO: 6, “HDAg gtp 1B-pool 3” and “HDAg gtp 1B-pool 4”), HDAg genotype 2 A (SEQ ID NO: 7, “HDAg gtp 2C-pool 5” and “HDAg gtp 2C-pool 6”) and HDAg genotype 2 B (SEQ ID NO: 8, “HDAg gtp 2D-pool 7” and “HDAg gtp 2D-pool 8”). Mice sacrificed 6 weeks after first immunization and pooled splenocytes from each group were stimulated for 48 hours with the HDV peptide pools 1-8 corresponding to genotype 1 (pools 1-4) and genotype 2 (pools 5-8). Pools 1 and 2 of genotype 1 refer to sequence/isolate A while pools 3 and 4 correspond to sequence/isolate B. Similarly, pools 5 and 6 of genotype 2 refer to sequence/isolate C and pools 7 and 8 of genotype 2 refer to sequence/isolate D. Each pool contained 20 or 221 (for pools 1 and 5) 15-mer peptides with 10 aa overlap. Concanavalin A (“ConA”) was used as a positive control, and two ovalbumin peptides (“OVA Th” and “OVA CTL”) and growth medium (“media”) were used as negative controls. Each peptide-stimulated group were run in triplicates and bars show the mean number of IF65 spot forming cells (SFC) per 10⁶ cells with standard error. A cut-off was set at 100 SECS/10⁶ splenocytes. Concentrations of the antigens are provided.

FIGS. 4A-4C depict the quantification of anti-PreS1 IgG antibody titer in sera derived from mice immunized with HBV/HDV DNA compositions. Constructs Δ-1 to Δ-10 were tested for generation of IgG antibodies against PreS1A and PreS1B consensus sequences in mice (5 mice per group). FIGS. 4A-4B cover reactivity against PreS1 amino acids 2-48. FIG. 4C cover cross-reactivity against HBV (sub-) genotypes A1, A2, B, B2, C, D1, E1 and F.

FIGS. 5A-5C depict the quantification of interferon gamma (IFNγ) forming spots per 10⁶ cells on an ELISpot assay of a purified white blood cell population from sera derived from C57BL/6 or HLA-A2 transgenic HHD mice immunized with a Δ-4 DNA composition, or naïve C57BL/6 mice in response to exposure to various HBV or HDV antigens or peptides. Antigens include purified polypeptides comprising PreS1 A (SEQ ID NO: 11), PreS1 A (SEQ ID NO: 12), a pool comprising HDAg genotypes 1 A and 1 B (SEQ ID NOs: 5 and 6, “gtp 1-pool 1”, “gtp 1-pool 2”, “gtp 1-pool 3”, “gtp 1-pool 4”), a pool comprising HDAg genotypes 2 A and 2 B (SEQ ID NOs: 7 and 8, “gtp 2-pool B1”, “gtp 2-pool B2”, “gtp 2-pool B3”, “gtp 2-pool B4”), HDAg peptide fragment pools comprising peptides KLEDDNPWL, KLEEENPWL, and FPWDILFPA (“pep-3-pool”), and individual HDAg peptides KLEDDNPWL, KLEEENPWL, and FPWDILFPA. Concanavalin A (“ConA”) was used as a positive control, and two ovalbumin peptides (“OVA Th” and “OVA CTL”) and growth medium (“media”) were used as negative controls. Concentrations of the antigens are provided.

FIGS. 6A-6C depict the quantification of anti-PreS1 IgG titer in New Zealand white rabbits immunized with Δ-3 or Δ-4 DNA compositions. Serum from the rabbits were collected and tested by ELISA against the PreS1A and PreS1B consensus peptides (FIG. 6B). The vaccinated rabbit anti-sera also tested for cross-reactivity to HBV (sub-) genotypes A1, A2, B, B2, C, D1, E1, and F (FIG. 6C). Graph bars show the mean end anti-PreS1 titers for each group determined as the end last serum dilution giving an OD at 405 nm three times higher than the OD of non-immunized sera at the same dilution. The sera was titrated serially with six-fold dilutions starting at 1:60.

FIG. 6D shows the percentage of reactivity of D-4 vaccinated rabbit antisera against PreS1 of different HBV (sub-) genotypes. Six weeks old D-4 vaccinated rabbit antisera were tested for reactivity (at OD 405 nm) against HBV (sub-) genotypes D1, F, A1, C, A2, B, B2 and E1 by ELISA. Using individual 20 mer PreS1 peptides with ten aa overlap corresponding to each HBV type of aa 2-21, 12-31, 22-41, and 32-48, neutralizing epitopes mainly localized at aa 22-41 and 32-48 of genotype D1 as indicated by the highest percentage of reactivity, followed by (sub-) types C, E1 and A1 at the same aa region.

FIGS. 7A-7C depict the quantification of interferon gamma (IFNγ) forming spots per 10⁶ cells on an ELISpot assay of a purified white blood cell population from sera derived from C57BL/6 mice immunized with a Δ-4 DNA-only, Δ-7 protein-only, or Δ-4 DNA/Δ-8 protein prime/boost composition. Antigens include purified polypeptides comprising PreS1 A (SEQ ID NO: 11), PreS1 A (SEQ ID NO: 12), a pool comprising HDAg genotypes 1 A and 1 B (SEQ ID NOs: 5 and 6, “gtp 1-pool 1”, “gtp 1-pool 2”, “gtp 1-pool 3”, “gtp 1-pool 4”), and a pool comprising HDAg genotypes 2 A and 2 B (SEQ ID NOs: 7 and 8, “gtp 2-pool 5”, “gtp 2-pool 6”, “gtp 2-pool 7”, “gtp 2-pool 8”). Concanavalin A (“ConA”) was used as a positive control, and two ovalbumin peptides (“OVA Th” and “OVA CTL”), DMSO, and growth medium (“media”) were used as negative controls. Concentrations of the antigens are provided.

FIGS. 8A-8C depict the quantification of anti -PreS1 IgG titer in C57BL/6 mice immunized with HBV/HDV DNA-only, protein-only, or DNA prime/protein boost compositions.

FIG. 9 depicts the quantification of anti-PreS1 IgG titer in rabbits immunized with HBVIIIDV DNA-only, protein-only, or DNA prime/protein boost compositions.

FIG. 10A-10B depicts the protective effect against F1BV infection at 1, 2, 3, 4, 6 and 8 weeks after first inoculation, as determined based on the HBV titers at each time point. Each line indicates one individual mouse (FIG. 10A). Two negative control mice (grey lines) received non-immunized IgG and three mice (red lines) received D4 PreS1 IgG. One mouse of the PreS1-IgG treated group died at week 4, thus only measurements on week 1, 2, and 3 are available for this mouse. There were no significant differences between the groups with respect to serum levels of alanine transferase, asparagine transferase, alkaline phosphatase, or bilirubin (FIG. 10B).

FIG. 11A-D depict the assessment of the D-7 and D-8 peptide mixture (10 μg each for administration in mice) with different adjuvants. QS-21, MF59, and alum adjuvants were tested. D-4 DNA compositions administered intramuscularly with electroporation was used as control. FIG. 11A shows the dosing schedule and exemplary end titers with the tested adjuvants. FIG. 11B shows % reactivity in individual mice of each condition assessed by ELISA. The x-axis (“1, 3, 10, 30, 0”) corresponds to ID numbers of individual mice. FIG. 11C shows IFNγ activation of splenocytes by HMI PreS1 and HDV antigen consensus peptides as assessed by ELISpot. FIG. 11D shows end-point PreS1 titers against PreS1A and PreS1B peptides.

FIG. 12A-D depict a comparison of D-7 and D-8 peptide mixture only, D-7+D-8 fusion peptide only, and D-4 DNA prime and D-7 and D-8 peptide mixture boost compared to D-4 DNA only and naïve controls. FIG. 12A shows IFNγ activation of splenocytes by HBV PreS1 and HDV antigen consensus peptides as assessed by ELISpot. FIG. 12B shows antibody levels against PreS1A assessed 2 weeks after the first round of administration. FIG. 12C shows antibody levels against PreS 1 A assessed 2 weeks after the second round of administration. FIG. 12D shows antibody levels against PreS1B assessed 2 weeks after the second round of administration. The legends for FIGS. 12B-D correspond to ID numbers of individual mice.

### DETAILED DESCRIPTION

Despite preventative vaccines and antiviral therapies, chronic hepatitis B virus (HBV) infection currently affects over 250 million people across the globe. One million chronic carriers die every year due to liver related complications caused by HBV, such as liver cirrhosis and eventually hepatocellular carcinoma (HCC). The hepatitis D virus (HDV), an RNA satellite virus to HBV that “steals” the surface antigen from HBV (HBsAg), co-infects 15-25 million of HBV carriers globally and worsens disease progression. Until now, there is no effective functional cure for chronic HBV or HDV infection. The current standard of care therapy for HBV consists of nucleoside analogues (NAs) that inhibit the reverse transcriptase (RT) function of the HBV polymerase. This prevents viral maturation by blocking the synthesis of the partially dsDNA inside the capsid. Thus, NAs only suppress the viral replication during therapy. This is due to the fact that blocking of RI neither affects protein production (including HBsAg) and release, nor synthesis of the covalently closed circular DNA, the main cause for HBV persistence. A life-long NA reduces, but does not eliminate, the risk of HCC. A schedule of at least one-year pegylated interferon (IFN)-alpha is the currently recommended treatment for chronic HDV; however, sustained response rates are rare. Combination treatment with pegylated IFN-alpha and NAs has been shown to have limited efficacy against HDV and HBV.

HBV uses several strategies in order to evade the host immune response. The chronic presence of HBV proteins induces a T cell dysfunction. HBV infected cells overproduce sub-viral HBsAg particles mainly containing small HBsAg (SHBsAg) to block the neutralizing antibody population directed to SHBsAg. This ensures survival of viral particles whose surface are denser with the middle HBsAg (MHBsAg; containing S and PreS2) and large HBsAg (LHBsAg; containing S, PreS2 and PreS1) proteins. Importantly, the PreS1 domain is responsible for binding to the Na⁺-taurocholate co-transporting polypeptide (NTCP) receptor for HBV hepatocytes. Thus, an obvious way to target infectious HBV particles and prevent the infection of new hepatocytes would be to raise antibodies to the PreS1 domain of the virus.

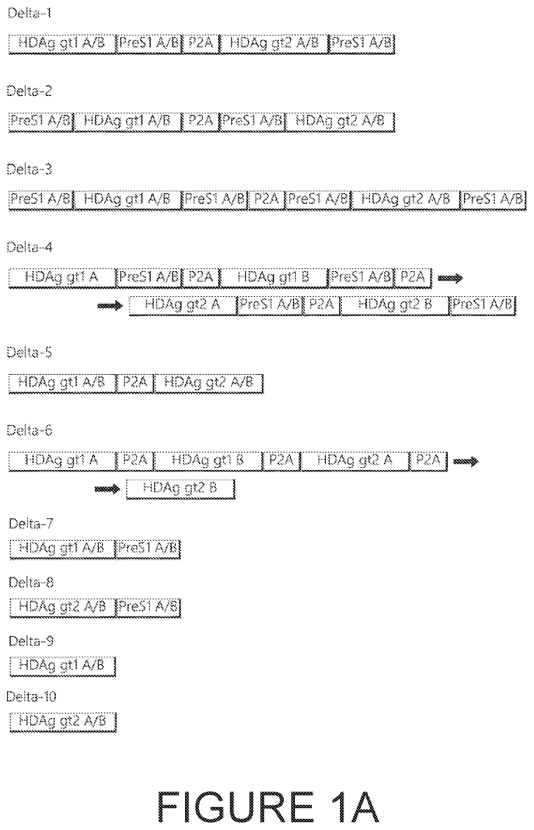

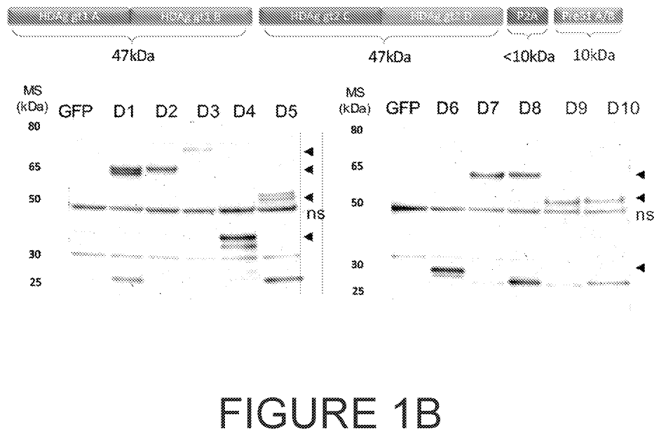

As disclosed herein, to build an immunotherapy targeting both HBV and HDV infections to induce production of PreS1 antibodies and T cells specific for HBV and HDV, chimeric genes containing PreS1 and the large HDV antigen were generated in different combinations (FIG. 1A). The advantage of linking PreS1 to HDAg is that HDAg will act as a heterologous I cell epitope carrier in patients that are mono-infected by HBV. Thus, these HDAg-specific T cells support a sustained endogenous production of PreS1 antibodies that block viral entry and bypass the need for HBV-specific T cells. In fact, >90% of HBV carriers are mono-infected with HBV, and in these patients, the heterologous HDAg will prime healthy naïve T cells that support priming of HBV-specific responses. In addition, it is likely that the HDAg-specific T cells and PreS1 antibodies prevent HDV superinfection in these patients. To induce both neutralizing antibodies and T cells, genetic immunization was used, as this strategy has shown to activate immune response to HBV. Overall, this virus entry-blocking strategy complements the maturation inhibitors and the capsid assembly inhibitors, currently under development, in order to achieve sustainable off-therapy responses against HBD and/or HDV infection.

Embodiments provided herein related to immunogenic compositions or product combinations of engineered hepatitis B (HBV) and hepatitis D (HDV) nucleic acids, genes, peptides, or proteins that can be used to illicit an immune response against an HBV or HDV infection. The use of chimeric genes and chimeric proteins comprising FIBV and FIDV nucleic acids, genes, peptides, or proteins has been characterized, for example, in WO 2017/132332, hereby expressly incorporated by reference in its entirety.

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

Unless defined otherwise, all technical and scientific terms used herein have the same meaning as is commonly understood by one of ordinary skill in the art. All patents, applications, published applications and other publications referenced herein are expressly incorporated by reference in their entireties unless stated otherwise. In the event that there are a plurality of definitions for a term herein, those in this section prevail unless stated otherwise.

The articles “a” and “an” are used herein to refer to one or to more than one (for example, at least one) of the grammatical object of the article. By way of example, “an element” means one element or more than one element.

The terms “about” or “around” as used herein refer to a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

Throughout this specification, unless the context requires otherwise, the words “comprise,” “comprises,” and “comprising” will be understood to imply the inclusion of a stated step or element or group of steps or elements but not the exclusion of any other step or element or group of steps or elements.

By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The practice of the present disclosure will employ, unless indicated specifically to the contrary, conventional methods of molecular biology and recombinant DNA techniques within the skill of the art, many of which are described below for the purpose of illustration. Such techniques are explained fully in the literature. See, e.g., Sambrook, et al, Molecular Cloning: A Laboratory Manual (3rd Edition, 2000); DNA Cloning: A Practical Approach, vol. 1 & II (D. Glover, ed.); Oligonucleotide Synthesis (N. Gait, ed., 1984); Oligonucleotide Synthesis: Methods and Applications (P. Herdewijn, ed., 2004); Nucleic Acid Hybridization (B. Hames & S. Higgins, eds., 1985); Nucleic Acid Hybridization: Modern Applications (Buzdin and Lukyanov, eds., 2009); Transcription and Translation (B. Hames & S. Higgins, eds., 1984); Animal Cell Culture (R. Freshney, ed., 1986); Freshney, R. I. (2005) Culture of Animal Cells, a Manual of Basic Technique, 5th Ed, Hoboken, N.J., John Wiley & Sons; B. Perbal, A Practical Guide to Molecular Cloning (3rd Edition 2010); Farrell, R., RNA Methodologies: A Laboratory Guide for Isolation and Characterization (3rd Edition 2005).

The term “purity” of any given substance, compound, or material as used herein refers to the actual abundance of the substance, compound, or material relative to the expected abundance. For example, the substance, compound, or material may be at least 80, 85, 90, 91, 92, 93, 94, 95, 96, 97, 98, 99, or 100% pure, including all decimals in between. Purity may be affected by unwanted impurities, including but not limited to side products, isomers, enantiomers, degradation products, solvent, carrier, vehicle, or contaminants, or any combination thereof. Purity can be measured technologies including but not limited to chromatography, liquid chromatography, gas chromatography, spectroscopy, UV-visible spectrometry, infrared spectrometry, mass spectrometry, nuclear magnetic resonance, gravimetry, or titration, or any combination thereof.

The terms “function” and “functional” as used herein refer to a biological, enzymatic, or therapeutic function.

The phrases “effective amount” or “effective dose” as used herein refers to an amount sufficient to achieve the desired result and accordingly wilt depend on the ingredient and its desired result. Nonetheless, once the desired effect is known, determining the effective amount is within the skill of a person skilled in the art.

In general, the “error bars” provided in the figures represent the standard error of the mean value.

“Formulation” and “composition” as used interchangeably herein are equivalent terms referring to a composition of matter for administration to a subject.

The term “isolated” as used herein refers to material that is substantially or essentially free from components that normally accompany it in its native state. For example, an “isolated cell,” as used herein, includes a cell that has been purified from the milieu or organisms in its naturally occurring state, a cell that has been removed from a subject or from a culture, for example, it is not significantly associated with in vivo or in vitro substances.

The term “subject” as used herein has its ordinary meaning as understood in light of the specification and refers to an animal that is the object of treatment, inhibition, or amelioration, observation or experiment. “Animal” has its ordinary meaning as understood in light of the specification and includes cold- and warm-blooded vertebrates and/or invertebrates such as fish, shellfish, or reptiles and, in particular, mammals. “Mammal” has its ordinary meaning as understood in light of the specification, and includes but is not limited to mice, rats, rabbits, guinea pigs, dogs, cats, sheep, goats, cows, horses, primates, such as humans, monkeys, chimpanzees, or apes. In some embodiments, the subject is human.

Some embodiments disclosed herein related to selecting a subject or patient in need. In some embodiments, a patient is selected who is in need of treatment of a viral infection. In some embodiments, a patient is selected who has previously been treated for a viral infection. In some embodiments, a patient is selected who has previously been treated for being at risk of a viral infection. In some embodiments, a patient is selected who has developed a recurrence of a viral infection. In some embodiments, a patient is selected who has developed resistance to therapies for a viral infection. In some embodiments, a patient is selected who may have any combination of the aforementioned selection criteria.

The terms “treating,” “treatment,” “therapeutic,” or “therapy” as used herein has its ordinary meaning as understood in light of the specification, and do not necessarily mean total cure or abolition of the disease or condition. The term “treating” or “treatment” as used herein (and as well understood in the art) also means an approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delaying or slowing of disease progression, amelioration or palliation of the disease state, diminishment of the reoccurrence of disease, and remission, whether partial or total and whether detectable or undetectable. “Treating” and “treatment” as used herein also include prophylactic treatment. Treatment methods comprise administering to a subject a therapeutically effective amount of an active agent. The administering step may consist of a single administration or may comprise a series of administrations. The compositions are administered to the subject in an amount and for a duration sufficient to treat the patient. The length of the treatment period depends on a variety of factors, such as the severity of the condition, the age and genetic profile of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required.

The term “inhibit” as used herein has its ordinary meaning as understood in light of the specification, and may refer to the reduction or prevention of a viral infection. The reduction can be by 10%, 20%, 30%, 40%, 50%, 60%, 70%, 80%, 90%, or 100%, or an amount that is within a range defined by any two of the aforementioned values. As used herein, the term “delay” has its ordinary meaning as understood in light of the specification, and refers to a slowing, postponement, or deferment of an event, such as a viral infection, to a time which is later than would otherwise be expected. The delay can be a delay of 0%, 10%, 20%, 30%, 40%, 50%, 60%, 70%, 80%, 90%, 100%, or an amount within a range defined by any two of the aforementioned values. The terms inhibit and delay may not necessarily indicate a 100% inhibition or delay. A partial inhibition or delay may be realized.

The term “immunogenic composition” as used herein refers to a substance or mixture of substances, including but not limited to antigens, epitopes, nucleic acids, peptides, polypeptides, proteins, polysaccharides, lipids, haptens, toxoids, inactivated organisms, or attenuated organisms, or any combination thereof, intended to elicit an immune response when administered to a host. The immune response includes both an innate and adaptive immune response, the latter of which establishes a lasting immunological memory through cells such as memory T cells and memory B cells. The antibodies created during the initial immune response to the immunogenic composition can be produced in subsequent challenges of the same antigens, epitopes, nucleic acids, peptides, polypeptides, proteins, polysaccharides, lipids, haptens, toxoids, inactivated organisms, or attenuated organisms, or a live organism or pathogen that exhibits the antigens, epitopes, nucleic acids, peptides, polypeptides, proteins, polysaccharides, lipids, haptens, or toxoids or any combination thereof. In this manner, the immunogenic composition may serve as a vaccine against a specific pathogen. Immunogenic compositions may also include one or more adjuvants to stimulate the immune response and increase the efficacy of protective immunity.

The term “product combination” as used herein refers to set of two or more individual compounds, substances, materials, or compositions that can be used together for a unified function. In some embodiments, a product combination comprises at least one nucleic acid composition and at least one polypeptide composition that are used together to elicit an immune response when administered to a host, optionally to a greater degree than would be elicited if only one composition type were to be administered.

The terms “nucleic acid” or “nucleic acid molecule” as used herein refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, or phosphoramidate. The term “nucleic acid molecule” also includes so-called “peptide nucleic acids,” which comprise naturally-occurring or modified nucleic acid bases attached to a polyamide backbone. Nucleic acids can be either single stranded or double stranded. “Oligonucleotide” can be used interchangeable with nucleic acid and can refer to either double stranded or single stranded DNA or RNA. A nucleic acid or nucleic acids can be contained in a nucleic acid vector or nucleic acid construct (e.g. plasmid, virus, bacteriophage, cosmid, fosmid, phagemid, bacterial artificial chromosome (BAC), yeast artificial chromosome (YAC), or human artificial chromosome (HAC)) that can be used for amplification and/or expression of the nucleic acid or nucleic acids in various biological systems. Typically, the vector or construct will also contain elements including but not limited to promoters, enhancers, terminators, inducers, ribosome binding sites, translation initiation sites, start codons, stop codons, polyadenylation signals, origins of replication, cloning sites, multiple cloning sites, restriction enzyme sites, epitopes, reporter genes, selection markers, antibiotic selection markers, targeting sequences, peptide purification tags, or accessory genes, or any combination thereof.

A nucleic acid or nucleic acid molecule can comprise one or more sequences encoding different peptides, polypeptides, or proteins. These one or more sequences can be joined in the same nucleic acid or nucleic acid molecule adjacently, or with extra nucleic acids in between, e.g. linkers, repeats or restriction enzyme sites, or any other sequence that is 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths. The term “downstream” on a nucleic acid as used herein refers to a sequence being after the 3′-end of a previous sequence, on the strand containing the encoding sequence (sense strand) if the nucleic acid is double stranded. The term “upstream” on a nucleic acid as used herein refers to a sequence being before the 5′-end of a subsequent sequence, on the strand containing the encoding sequence (sense strand) if the nucleic acid is double stranded. The term “grouped” on a nucleic acid as used herein refers to two or more sequences that occur in proximity either directly or with extra nucleic acids in between, e.g. linkers, repeats, or restriction enzyme sites, or any other sequence that is 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths, but generally not with a sequence in between that encodes for a functioning or catalytic polypeptide, protein, or protein domain.

The term “codon optimized” regarding a nucleic acid as used herein refers to the substitution of codons of the nucleic acid to enhance or maximize translation in a host of a particular species without changing the polypeptide sequence based on species-specific codon usage biases and relative availability of each aminoacyl-tRNA in the target cell cytoplasm. Codon optimization and techniques to perform such optimization is known in the art. Programs containing algorithms for codon optimization are known to those skilled in the art. Programs can include, for example, OptimumGene, GeneGPS® algorithms, etc. Additionally, synthetic codon optimized sequences can be obtained commercially for example from Integrated DNA Technologies and other commercially available DNA sequencing services. Those skilled in the art will appreciate that gene expression levels are dependent on many factors, such as promoter sequences and regulatory elements. As noted for most bacteria, small subsets of codons are recognized by tRNA species leading to translational selection, which can be an important limit on protein expression. In this aspect, many synthetic genes can be designed to increase their protein expression level.

The nucleic acids described herein comprise nucleobases. Primary, canonical, natural, or unmodified bases are adenine, cytosine, guanine, thymine, and uracil. Other nucleobases include but are not limited to purines, pyrimidines, modified nucleobases, 5-methylcytosine, pseudouridine, dihydrouridine, inosine, 7-methylguanosine, hypoxanthine, xanthine, 5,6-dihydrouracil, 5-hydroxymethylcytosine, 5-bromouracil, isoguanine, isocytosine, aminoallyl bases, dye-labeled bases, fluorescent bases, or biotin-labeled bases.

The terms “peptide”, “polypeptide”, and “protein” as used herein refers to macromolecules comprised of amino acids linked by peptide bonds. The numerous functions of peptides, polypeptides, and proteins are known in the art, and include but are not limited to enzymes, structure, transport, defense, hormones, or signaling. Peptides, polypeptides, and proteins are often, but not always, produced biologically by a ribosomal complex using a nucleic acid template, although chemical syntheses are also available. By manipulating the nucleic acid template, peptide, polypeptide, and protein mutations such as substitutions, deletions, truncations, additions, duplications, or fusions of more than one peptide, polypeptide, or protein can be performed. These fusions of more than one peptide, polypeptide, or protein can be joined in the same molecule adjacently, or with extra amino acids in between, e.g. linkers, repeats, epitopes, or tags, or any other sequence that is 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths.

In some embodiments, the nucleic acid or peptide sequences presented herein and used in the examples are functional in various biological systems including but not limited to humans, mice, rabbits, E. coli, yeast, and mammalian cells. In other embodiments, nucleic acid or peptide sequences sharing 0%, 10%, 20%, 30%, 40%, 50%, 60%, 70%, 80%, 90%, 91%, 92%, 93%, 94%, 95%, 96%, 97%, 98%, 99%, or 100% similarity, or any percentage within a range defined by any two of the aforementioned percentages similarity to the nucleic acid or peptide sequences presented herein and used in the examples can also be used with no effect on the function of the sequences in biological systems. As used herein, the term “similarity” refers to a nucleic acid or peptide sequence having the same overall order of nucleotide or amino acids, respectively, as a template nucleic acid or peptide sequence with specific changes such as substitutions, deletions, repetitions, or insertions within the sequence. In some embodiments, two nucleic acid sequences sharing as low as 0%, 10%, 20%, 30%, 40%, 50%, 60%, 70%, 80%, 90%, 91%, 92%, 93%, 94%, 95%, 96%, 97%, 98%, or 99% similarity can encode for the same polypeptide by comprising different codons that encode for the same amino acid during translation.

The term “recombinantly expressed” as used herein refers to the production of proteins in optimized or adapted biological systems. These systems provide advantages over protein expression in a natural host, including but not limited to high expression (overexpression), ease of purification, ease of transformation, inducibility, low cost, or stability of the protein. In some embodiments, proteins are expressed in mammalian, bacteria, yeast, insect, or cell-free recombinant expression systems. Each system has its own advantages or disadvantages. For example, bacterial expression systems are highly optimized for overexpression, but may cause misfolding or aggregation of the produced protein, yeast systems are useful when post-translational modifications are necessary, and insect and mammalian systems are useful for proper RNA splicing that occurs in higher-order organisms. In some embodiments, Δ-7, Δ-8, and other recombinant polypeptides are produced and purified from mammalian, human, primary, immortalized, cancer, stem, fibroblasts, human embryonic kidney (HEIS) 293, Chinese Hamster Ovary (CHO), bacterial, Escherichia coli, yeast, Saccharomyces cerevisiae, Pichia pastoris, insect, Spodoptera frugiperda Sf9, or S. frugiperda Sf21 cells, or in a cell-free system. In some embodiments, expression genes, vectors, or constructs are delivered to the recombinant expression systems in the form of plasmids, bacteriophages, viruses, adeno-associated viruses (AAVs), baculovirus, cosmids, fosmids, phagemids, BACs, YACs, or HACs. For more discussion on recombinant expression systems, see Gomes et al. “An Overview of Heterologous Expression Host Systems for the Production of Recombinant Proteins” ((2016) Adv. Anim. Vet. Sci. 4(7):346-356), hereby expressly incorporated by reference in its entirety.

The term “HDAg” as used herein refers the hepatitis D antigen gene or protein. A small (24 kDa) and large (27 kDa, 213 amino acids excluding the start methionine) isoform exist for HDAg and are translated from the same open reading frame on the 1.-IDV genome. Deamination of the adenosine in a UAG stop codon at codon 196 of the coding sequence allows for translation to continue and produce the large isoform. Unless expressly stated otherwise, the embodiments described herein comprise the large isoform of HDAg. In some embodiments, the HDAg sequences comprise at least one of four different HDAg strain sequences: “HDAg genotype 1 A”, “HDAg genotype 1 B”, “HDAg genotype 2 A”, or “HDAg genotype 2 B”. In some embodiments, the nucleic acid sequence encoding at least one HDAg polypeptide comprises the nucleic acid sequence of HDAg genotype 1 A (SEQ ID NO: 1), HDAg genotype 1 B (SEQ ID NO: 2), HDAg genotype 2 A (SEQ ID NO: 3), or HDAg genotype 2 B (SEQ ID NO: 4). In some embodiments, the polypeptide comprising at least one HDAg polypeptide comprises the polypeptide sequence of HDAg genotype 1 A (SEQ ID NO: 5), HDAg genotype 1 B (SEQ ID NO: 6), HDAg genotype 2 A (SEQ ID NO: 7), or HDAg genotype 2 B (SEQ ID NO: 8).

The term “PreS1” as used herein refers to a segment of the N-terminal domain on the large surface antigen of HBV (HBsAg). A 47 amino acid long PreS1 segment of the 108-119 amino acid long N-terminal domain of the large HBsAg is effective in eliciting an immune response and generating high titer of anti-PreS1 anti-HBV antibodies in mammalian models. In some embodiments, the PreS1 sequences comprise at least one of two different PreS1 consensus sequences: “PreS1 A” and/or “PreS1 B”. In some embodiments, the nucleic acid sequence encoding at least one PreS1 polypeptide comprises the nucleic acid sequence of PreS1 A (SEQ ID NO: 9) or PreS1 B (SEQ ID NO: 10). In some embodiments, the polypeptide comprising at least one PreS1 polypeptide comprises the polypeptide sequence of PreS1 A (SEQ ID NO: 11) or PreS1 B (SEQ ID NO: 12).

In some embodiments, the PreS1 A and PreS1 B consensus sequences of HBV are obtained or derived from sequence similarity of PreS1 in known genotypes of HBV. There are ten known or prevalent HMI genotypes (genotypes A, B, C, D, E, F, G, H, I, and J) exhibiting up to or about 8% nucleotide differences in genomic sequence. Of these, there are additional sub-genotypes exhibiting up to or about 4%-8% nucleotide differences in genomic sequence. Sub-genotypes of HBV include but are not limited to A1, A2, A3, A4, A5, A6, A7, B2, B3, B4, B5, B6, B7, B9, C1, C2, C3, C4, C5, C6, C7, C8, C9, C10, D1, D2, D3, D4, D5, D6, D7, F1, F2, F2a, F3, or F4. For more discussion on HBV genotypes, see Sunbul “Hepatitis B virus genotypes: Global distribution and clinical importance” ((2014) World. J. Gastroenterology, 20(18):5427-5434, hereby expressly incorporated by reference in its entirety.

The terms “autocatalytic peptide cleavage site” or “2A peptide” as used herein refer to a peptide sequence that undergo cleavage of a peptide bond between two constituent amino acids, resulting in separation of the two proteins that flank the sequence. The cleavage is believed to be a result of a ribosomal “skipping” of the peptide bond formation between the C-terminal proline and glycine in the 2A peptide sequence. Four autocatalytic peptide cleavage site sequences identified to date have seen substantial use in biomedical research: foot-and-mouth disease virus 2A (F2A); equine rhinitis A virus WRAY) 2A (E2A); porcine teschovirus-1 2A (P2A), and Thosea asigna virus 2A (T2A). In some embodiments, the P2A autocatalytic peptide cleavage site nucleic acid (SEQ ID NO: 13) and polypeptide (SEQ ID NO: 14) sequences are used.

The term “HBeAg” as used herein refers to an HBV antigen protein found between the nucleocapsid core and lipid envelope of the virus. HBeAg produced in a host is secreted into the blood serum and is a good marker for an active HBV infection. Quantification of in vitro HBeAg secretion in a cell culture model can be used to assess effect of biological or pharmaceutical compounds or compositions on HBV infectivity.

The term “excipient” has its ordinary meaning as understood in light of the specification, and refers to other substances, compounds, or materials found in an immunogenic composition or vaccine. Excipients with desirable properties include but are not limited to preservatives, adjuvants, stabilizers, solvents, buffers, diluents, solubilizing agents, detergents, surfactants, chelating agents, antioxidants, alcohols, ketones, aldehydes, ethylenediaminetetraacetic acid (EDTA), citric acid, salts, sodium chloride, sodium bicarbonate, sodium phosphate, sodium borate, sodium citrate, potassium chloride, potassium phosphate, magnesium sulfate sugars, dextrose, fructose, mannose, lactose, galactose, sucrose, sorbitol, cellulose, serum, amino acids, polysorbate 20, polysorbate 80, sodium deoxycholate, sodium taurodeoxycholate, magnesium stearate, octylphenol ethoxylate, benzethonium chloride, thimerosal, gelatin, esters, ethers, 2-phenoxyethanol, urea, or vitamins, or any combination thereof. Some excipients may be in residual amounts or contaminants from the process of manufacturing the immunogenic composition or vaccine, including but not limited to serum, albumin, ovalbumin, antibiotics, inactivating agents, formaldehyde, glutaraldehyde, β-propiolactone, gelatin, cell debris, nucleic acids, peptides, amino acids, or growth medium components or any combination thereof. The amount of the excipient may be found in an immunogenic composition or vaccine at a percentage of 0%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 20%, 30%, 40%, 50%, 60%, 70%, 80%, 90%, 95%, 100% w/w or any percentage by weight in a range defined by any two of the aforementioned numbers.

The term “adjuvant” as used herein refers to a substance, compound, or material that stimulates the immune response and increase the efficacy of protective immunity and is administered in conjunction with the immunogenic antigen, epitope, or composition. Adjuvants serve to improve immune responses by enabling a continual release of antigen, up-regulation of cytokines and chemokines, cellular recruitment at the site of administration, increased antigen uptake and presentation in antigen presenting cells, or activation of antigen presenting cells and inflammasomes. Commonly used adjuvants include but are not limited to alum, aluminum salts, aluminum sulfate, aluminum hydroxide, aluminum phosphate, calcium phosphate hydroxide, potassium aluminum sulfate, oils, mineral oil, paraffin oil, oil-in-water emulsions, detergents, MF59®, squalene, AS03, α-tocopherol, polysorbate 80, AS04, monophosphoryl lipid A, virosomes, nucleic acids, polyinosinic:polycytidylic acid, saponins, QS-21, proteins, flagellin, cytokines, chemokines, IL-1, IL-2, IL-12, IL-15, IL-21, imidazoquinolines, CpG oligonucleotides, lipids, phospholipids, dioleoyl phosphatidylcholine (DOPC), trehalose dimycolate, peptidoglycans, bacterial extracts, lipopolysaccharides, or Freund's Adjuvant, or any combination thereof.

The terms “prime” and “boost” as used herein related to separate immunogenic compositions used in a heterologous prime-boost immunization approach. Immunizations or vaccines commonly require more than one administration of an immunogenic composition to induce a successful immunity against a target pathogen in a host. Compared to this homologous approach where the same composition is provided for all administrations, a heterologous prime-boost administration may be more effective in establishing robust immunity with greater antibody levels and improved clearing or resistance against some pathogens such as HBV or HDV. In a heterologous prime-boost administration, at least one prime dose comprising one type of immunogenic composition is first provided. After the at least one prime dose is provided, at least one boost dose comprising another type of immunogenic composition is then provided. Administration of the at least one boost dose is performed at least 1, 2, 3, 4, 5, 6, 7, 8, 9, 10, 11, 12, 24, 36, or 48 days or weeks after the at least one prime dose is administered or within a range of time defined by any two of the aforementioned time points e.g., within 1-48 days or 1-48 weeks. In some embodiments, the prime dose comprises a nucleic acid (e.g. DNA or RNA) that encodes for one or more antigens or epitopes, and the boost dose comprises a polypeptide that comprises one or more antigens or epitopes. In the host, the nucleic acid prime is translated in vivo to elicit an immune reaction and causes a greater response against the subsequent polypeptide boost. In some embodiments, the nucleic acid prime comprises sequences that encodes for at least one HDAg polypeptide, at least one PreS1 peptide, and at least one autocatalytic peptide cleavage site. In some embodiments, the polypeptide boost comprises at least one HDAg polypeptide and at least one PreS1 polypeptide.

In some embodiments, administration of the nucleic acid prime and polypeptide boost comprising HBV and HDV components in an experimental organism results in greater anti-HDAg, anti-PreS1, anti-HBV, or anti-HDV antibody titer at a ratio of 1, 2, 3, 4, 5, 6, 7, 8, 9, 10, 50, 100, 150, 200, 300, 400, 500, 600, 700, 800, 900, 1000, 5000, 10000, 100000, or 1000000 or any ratio within a range defined by any two of the aforementioned ratios compared to nucleic acid-only or polypeptide-only immunized, or unimmunized control organisms, quantified by techniques known in the art such as ELISA. In some embodiments, administration of the nucleic acid prime and polypeptide boost comprising HBV and HDV components in an experimental organism results in serum that neutralizes HBV or HDV infectivity in vitro more effectively and reduces the incidence of infection to a ratio of 0.00001, 0.00005, 0.0001, 0.0005, 0.001, 0.005, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9. or 1.0 or any ratio within a range defined by any two of the aforementioned ratios compared to sera from nucleic acid-only or polypeptide-only immunized, or unimmunized control organisms. In some embodiments, administration of the nucleic acid prime and polypeptide boost comprising HBV and HDV components in an experimental organism results in a greater number of interferon gamma (IFNγ)-positive cells (e.g. T cells) at a ratio of 1, 2, 3, 4, 5, 6, 7, 8, 9, 10, 20, 30, 40, 50, 60, 70, 80, 90, 100, 110, 120, 130, 140, 150, 200, 250, 300, 350, 400, 450, 500, 550, 600, 650, 700, 750, 800, 850, 900, 950, 1000, 5000, or 10000, or any ratio within a range defined by any two of the aforementioned ratios compared to nucleic acid-only or polypeptide-only immunized, or unimmunized control organisms.

In some embodiments, the immunogenic compositions or product combinations are administered with an adjuvant. In some embodiments, the immunogenic compositions or product combinations are administered enterally, orally, intranasally, parenterally, subcutaneously, intramuscularly, intradermally, or intravenously or any combination thereof. In some embodiments, the immunogenic compositions or product combinations are administered in conjunction with an antiviral therapy compound known to have an effect against HBV or HDV, including but not limited to entecavir, tenofovir, lamivudine, adefovir, telbivudine, emtricitabine, interferon-α, pegylated interferon-α, or interferon alfa-2b, or any combination thereof.

The terms “in vivo electroporation”, “electroporation”, and “EP” as used herein refers to the delivery of genes, nucleic acids, DNA, RNA, proteins, or vectors into cells of living tissues or organisms using electrical currents using techniques known in the art. Electroporation can be used as an alternative to other methods of gene transfer such as viruses (transduction), lipofection, gene gun (biolistics), microinjection, vesicle fusion, or chemical transformation. Electroporation limits the risk of immunogenicity and detrimental integration or mutagenesis of the cell genome. DNA vectors such as plasmids are able to access the cell nucleus, enabling transcription and translation of constituent genes. In some embodiments, the genes, nucleic acids, DNA, RNA, proteins, or vectors are added to the target tissue or organism by subcutaneous, intramuscular, or intradermal injection. An electroporator then delivers short electrical pulses via electrodes placed within or proximal to the injected sample. As used herein, the term “im/EP” refers to in vivo electroporation of a sample delivered intramuscularly (“im”).

The term “uPA^{+/+}-SCID” as used herein refers to an immunodeficient mouse model used for studying liver diseases including hepatitis viral infections. These mice are homozygous for Prkdc^{scid}, causing deficiencies in functional T and B lymphocytes. Overexpression of urokinase-type plasminogen activator (uPA) also causes severe liver cytotoxicity and hepatic insufficiency during development. Subsequent transplantation and engraftment of human liver tissue to these mice results in a model ideal for studying hepatic illnesses of humans. For more discussion on uPA^{+/+}-SCID mice, see Meuleman et al. “The human liver-uPA-SCID mouse: A model for the evaluation of antiviral compounds against HBV and HCV” ((2008) Antiviral Research 80(3):231-238), hereby expressly incorporated by reference in its entirety.

The term “% w/w” or “% wt/wt” as used herein has its ordinary meaning as understood in light of the specification and refers to a percentage expressed in terms of the weight of the ingredient or agent over the total weight of the composition multiplied by 100. The term “% v/v” or “% vol/vol” as used herein has its ordinary meaning as understood in the light of the specification and refers to a percentage expressed in terms of the liquid volume of the compound, substance, ingredient, or agent over the total liquid volume of the composition multiplied by 100.

The invention is generally disclosed herein using affirmative language to describe the numerous embodiments. The invention also includes embodiments in which subject matter is excluded, in full or in part, such as substances or materials, method steps and conditions, protocols, or procedures.

### Immunogenic Compositions and Product Combinations

Disclosed herein are immunogenic compositions or product combinations. In some embodiments, these immunogenic compositions or product combinations are intended to induce an immunogenic response against a particular antigen. In some embodiments, the immunogenic compositions or product combinations comprise (a) a nucleic acid comprising at least one nucleic acid sequence encoding hepatitis D antigen (HDAg) and at least one nucleic acid sequence encoding PreS1; and (b) a polypeptide comprising at least one HDAg polypeptide sequence and at least one PreS1 polypeptide sequence. In some embodiments, the at least one nucleic acid sequence encoding HDAg comprises SEQ ID NO: 1, SEQ ID NO: 2, SEQ ID NO: 3 or SEQ ID NO: 4, or any combination thereof. In some embodiments, the at least one nucleic acid sequence encoding PreS1 comprises SEQ ID NO: 9 or SEQ ID NO: 10 or both. In some embodiments, the nucleic acid is configured such that each HDAg nucleic acid sequence is grouped with a PreS1 nucleic acid sequence, and wherein the PreS1 nucleic acid sequence is immediately downstream of the HDAg nucleic acid sequence. In some embodiments, the immunogenic compositions or product combinations further comprise at least one nucleic acid sequence encoding an autocatalytic peptide cleavage site, wherein the grouped HDAg and PreS1 nucleic acid sequences are separated by the at least one nucleic acid sequence encoding the autocatalytic peptide cleavage site. In some embodiments, the at least one nucleic acid sequence encoding the autocatalytic peptide cleavage site comprises a nucleic acid sequence selected from the group consisting of porcine teschovirus-1 2A (P2A), foot-and-mouth disease virus 2A (F2A), equine rhinitis A virus (ERAV) 2A (E2A) and Thosea asigna virus 2A (T2A) nucleic acid, and wherein each encoded autocatalytic peptide cleavage site may optionally include a GSG (glycine-serine-glycine) motif at its N-terminus. In some embodiments, the at least one nucleic acid sequence encoding the autocatalytic peptide cleavage site comprises SEQ ID NO: 13. In some embodiments, the nucleic acid is codon optimized for expression in a human. In some embodiments, the nucleic acid comprises a sequence having at least 80%, 85%, 90%, 95%, 99%, or 100% homology to SEQ ID NO: 15-24 or 35-36. In some embodiments, the nucleic acid comprises a sequence having at least 80%, 85%, 90%, 95%, 99%, or 100% homology to SEQ ID NO: 18, or SEQ ID NO: 35-36. In some embodiments, the at least one HDAg polypeptide comprises SEQ ID NO: 5, SEQ ID NO: 6, SEQ ID NO: 7 or SEQ ID NO: 8 or any combination thereof. In some embodiments, the at least one PreS1 polypeptide sequence comprises SEQ ID NO: 11 or SEQ ID NO: 12 or both. In some embodiments, the at least one PreS1 polypeptide sequence is downstream of the at least one HDAg polypeptide sequence. In some embodiments, the polypeptide comprises a sequence having at least 80%, 85%, 90%, 95%, 99%, or 100% homology to the sequence of SEQ ID NO: 25-34 or 37. In some embodiments, the polypeptide comprises a sequence having at least 80%, 85%, 90%, 95%, 99%, or 100% homology to the sequence of SEQ ID NO: 29, 31, 32, or 37. In some embodiments, the polypeptide is recombinantly expressed. In some embodiments, the polypeptide is recombinantly expressed in a mammalian, bacterial, yeast, insect, or cell-free system. In some embodiments, the immunogenic compositions or product combinations further comprise an adjuvant. In some embodiments, the adjuvant is alum, QS-21 or MF59, or any combination thereof. In some embodiments, the nucleic acid comprises DNA. In some embodiments, the nucleic acid is provided in a recombinant vector.

Also disclosed herein are methods of generating an immune response in a subject using an immunogenic composition or product combination. In some embodiments, the immunogenic composition or product combination is any one of the immunogenic compositions or product combinations disclosed herein. In some embodiments, the methods comprises administering to the subject at least one prime dose comprising the nucleic acid; and administering to the subject at least one boost dose comprising the polypeptide. In some embodiments, the at least one boost dose further comprises an adjuvant. In some embodiments, the adjuvant is alum, QS-21, or MF59, or any combination thereof. In some embodiments, the at least one boost dose is administered at least 1, 2, 3, 4, 5, 6, 7, 8, 9, 10, 11, 12, 24, 36, or 48 days or weeks after the at least one prime dose is administered or within a range of time defined by any two of the aforementioned time points e.g., within 1-48 days or 1-48 weeks. In some embodiments, the administration is provided enterally, orally, intranasally, parenterally, subcutaneously, intramuscularly, intradermally, or intravenously or any combination thereof. In some embodiments, the administration is performed in conjunction with an antiviral therapy. In some embodiments, the antiviral therapy comprises administration of entecavir, tenofovir, lamivudine, adefovir, telbivudine, emtricitabine, interferon-α, pegylated interferon-α, or interferon alfa-2b, or any combination thereof.

Also disclosed herein are immunogenic compositions or product combinations for use in the treatment or inhibition of hepatitis B or hepatitis D. In some embodiments, the immunogenic compositions or product combinations are any one of the immunogenic compositions or product combinations disclosed herein. In some embodiments, the immunogenic compositions or product combinations comprise (a) a nucleic acid comprising at least one nucleic acid sequence encoding hepatitis D antigen (MA), and at least one nucleic acid sequence encoding PreS1; and (b) a polypeptide comprising at least one HDAg polypeptide sequence and at least one PreS1 polypeptide sequence. In some embodiments, the at least one nucleic acid sequence encoding HDAg comprises SEQ ID NO: 1, SEQ ID NO: 2, SEQ ID NO: 3 or SEQ ID NO: 4. In some embodiments, the at least one nucleic acid sequence encoding PreS1 comprises SEQ ID NO: 9 or SEQ ID NO: 10 or both. In some embodiments, the nucleic acid is configured such that each HDAg nucleic acid sequence is grouped with a PreS1 nucleic acid sequence, and wherein the PreS1 nucleic acid sequence is immediately downstream of the HDAg nucleic acid sequence. In some embodiments, the immunogenic compositions or product combinations further comprise at least one nucleic acid sequence encoding an autocatalytic peptide cleavage site, wherein the grouped HDAg and PreS1 nucleic acid sequences are separated by the at least one nucleic acid sequence encoding the autocatalytic peptide cleavage site. In some embodiments, the at least one nucleic acid sequence encoding the autocatalytic peptide cleavage site comprises a nucleic acid sequence selected from the group consisting of porcine teschovirus-1 2A (P2A), foot-and-mouth disease virus 2A (F2A), equine rhinitis A virus (ERAV) 2A (E2A) and Thosea asigna virus 2A (12A) nucleic acid, and wherein each encoded autocatalytic peptide cleavage site may optionally include a GSG (glycine-serine-glycine) motif at its N-terminus. In some embodiments, the at least one nucleic acid sequence encoding the autocatalytic peptide cleavage site comprises SEQ ID NO: 13. In some embodiments, the nucleic acid is codon optimized for expression in a human. In some embodiments, the nucleic acid comprises a sequence having at least 80%, 85%, 90%, 95%, 99%, or 100% homology to SEQ ID NO: 15-24 or 35-36. In some embodiments, the nucleic acid comprises a sequence having at least 80%, 85%, 90%, 95%, 99%, or 100% homology to SEQ ID NO: 18, or SEQ ID NO: 35-36. In some embodiments, the at least one HDAg polypeptide comprises SEQ ID NO: 5, SEQ ID NO: 6, SEQ ID NO: 7 or SEQ ID NO: 8 or any combination thereof. In some embodiments, the at least one PreS1 polypeptide sequence comprises SEQ ID NO: 11 or SEQ ID NO: 12 or both. In some embodiments, the at least one PreS1 polypeptide sequence is downstream of the at least one HDAg polypeptide sequence. In some embodiments, the polypeptide comprises a sequence having at least 80%, 85%, 90%, 95%, 99%, or 100% homology to the sequence of SEQ ID NO: 25-34 or 37. In some embodiments, the polypeptide comprises a sequence having at least 80%, 85%, 90%, 95%, 99%, or 100% homology to the sequence of SEQ ID NO: 29, 31, 32, or 37. In some embodiments, the polypeptide is recombinantly expressed. In some embodiments, the polypeptide is recombinantly expressed in a mammalian, bacterial, yeast, insect, or cell-free system. In some embodiments, the immunogenic compositions or product combinations further comprise an adjuvant. In some embodiments, the adjuvant is alum, QS-21, or MF59, or any combination thereof. In some embodiments, the nucleic acid comprises DNA. In some embodiments, the nucleic acid is provided in a recombinant vector.

### EXAMPLES

Some aspects of the embodiments discussed above are disclosed in further detail in the following examples, which are not in any way intended to limit the scope of the present disclosure. Those in the art will appreciate that many other embodiments also fall within the scope of the invention, as it is described herein above and in the claims.

### Example 1: Methodology

### Animals

Female C57BL/6 (H-2^{b}) mice were obtained from Charles River Laboratories. Human leukocyte antigen A2 (HLA-A2) transgenic HHD mice were bred inhouse. All mice were 8-10 weeks old at the start of the experiments and maintained under standard conditions. uPA^{+/+}-SCID mice with humanized liver were produced and maintained. New Zealand White rabbits were purchased from commercial vendors.

### DNA Plasmids

Plasmids encoding for genotypes 1 and 2 of the L-HDAg and the PreS1 domain (aa 2-48) of the HBsAg, were used in this study as fusion constructs, optionally cleaved by a P2A, consisting of different combinations of HDAg/PreS1 sequences. The HDAg sequences of genotypes 1 and 2 obtained from four different clinical isolates; US-2 and CB, and 7/18/83 and TW2476, respectively. All genes were cloned into the pVAX1 backbone (Invitrogen, Carlsbad, Calif.) using restriction sites EcoR I and HindIII. Plasmids were grown in TOP10 E. coli cells (Life Technologies, Carlsbad, Calif.) and purified for in vivo injections using Qiagen Endofree DNA purification kit (Qiagen GmbH) following manufacturer's instructions. The correct gene size was confirmed by restriction enzyme digests using EcoR I and HindIII (Fast Digest, Thermo Fisher Scientific).

### Western Blot

Western Blot was essentially performed as generally known in the art. Hela cells were transfected with each pVAX1 D1-D10 DNA plasmids and pVAX1 with the reporter gene GFP as control, using Lipofectamine® 3000 Transfection Reagent (Thermo Fisher Scientific). For protein detection, serum from the D4 vaccinated rabbit diluted 1:1000 (primary antibody) and goat anti-Rabbit Immunoglobulins HRP 0.25 g/L (DAKO) diluted 1:4000 (secondary antibody) used. For Chemiluminescence detection, Pierce TM ECL Plus Western Blotting Substrate was used and images were collected with Gel Doc XR+ System (Biorad).

### Peptides

A total of 168 HDAg 15-mer peptides with 10 aa overlap were purchased from Sigma-Aldrich (St. Louis, Mo.). The 168 peptides were divided in 8 pools each containing 20 or 21 peptides. Four pools correspond to genotype 1 (pool 1₁₋₂₁, pool 2₂₂₋₄₂, pool 3₄₃₋₆₃ and pool 4₆₄₋₈₄) and four pools correspond to genotype 2 (pool 1₁₋₂₁, pool 2₂₂₋₄₂, pool 3₄₃₋₆₃ and pool 4₆₄₋₈₄) for sequences A, B, C and D with each sequence referring to each clinical isolate.

Two consensus sequences of the PreS1 HBsAg (PreS1A and PreS1B) consisting of 47 aa and 20-mer PreS1 peptides with 10 aa overlap for HBV (sub-) genotypes A1, A2, B, B2, C, D1, E1 and F were purchased from Sigma-Aldrich (St. Louis, Mo.). All peptides have passed QC (Sigma-Aldrich PEPscreen® Directory) and have purity >70%. OVA 257-264 CTL (SIINFEKL (SEQ ID NO: 38)) and OVA 323-339 Th (ISQAVHAAHAEINEAGR (SEQ ID NO: 39)) ovalbumin peptides were used as negative peptide controls while Concanavalin A (conA) purchased from Sigma Aldrich (St. Louis, Mo.) was used as positive control at final concentration of 0.5 μg/μL.

### Immunization Protocols for Evaluating Immunogenicity of HBV/HDV Plasmids in Mice and Rabbits

To evaluate the immunogenicity of the constructs in vivo, mice and rabbits were immunized essentially as described boosted at monthly intervals and sacrificed two weeks later for spleens and blood collection. In brief, female C57BL/6 mice (five per group) were immunized intramuscularly (i.m.) in the tibialis cranialis anterior (TA) muscle with 50 μg plasmid DNA in a volume of 50 μL in sterile PBS by regular needle (27G) injection followed by in vivo electroporation (EP) using the Cliniporator2 device (IGEA, Carpi, Italy). During in vivo electroporation, a 1 ms 600 V/cm pulse followed by a 400 ms 60 V/cm pulse pattern was used to facilitate better uptake of the DNA. Prior to vaccine injections, mice were given analgesic and kept under isoflurane anesthesia during the vaccinations. For studies in rabbits, two New Zealand White rabbits per group were immunized with 300 μg D3 and D4 DNA vaccines. Vaccines were administered by i.m. injection in 300 μL sterile PBS to the right TA muscle followed by in vivo EP.

### Detection of IFNγ Producing T Cells by Enzyme-Linked Immunospot Assay (ELISpot)

Two weeks after last vaccination, splenocytes from each immunized group of mice pooled (five mice/group) and tested for their ability to induce HBV/HDV-specific T cells based on IFN-γ secretion after peptide stimulation for 48 h as known in the art using a commercially available ELISpot assay (Mabtech, Nacka Strand, Sweden).

### Antibody Detection by MSA

Detection of mouse and rabbit IgG against PreS1 consensus and overlapping 20 mer-peptides (10 μg/mL) was performed using protocols known in the art. Antibody titers determined as endpoint serum dilutions at which the OD value at 405 nm is at least twice the OD of the negative control (non-immunized or control animal serum) at the same dilution.

HBV Neutralization Assay in Human-Liver uPA-SCID Mouse Model

HepG2-NTCP-A3 is a selected cell clone derived from the HepG2 cell expressing human NTCP as described previously. It was cultivated in DMEM medium supplemented with 10% fetal calf serum, 2 mM 1-glutamine, 50 U/mL penicillin, and 50 μg/mL streptomycin. During and after inoculation, 2.5% DMSO was added to the medium to enhance HBV infection and replication. HBV virus stock used for infection was prepared from HepAD38 cells by PEG precipitation as described. Cell culture medium between day 3-6 post infection were collected and diluted 1:5 with PBS for ELISA analysis of HBeAg quantification using commercial antibodies.

### Statistical Analysis

Data was analyzed using GraphPad Prism V.5 and V.8 software and Microsoft Excel V.16.13.1.

### Example 2: HBV and MDV Immunogenic Constructs

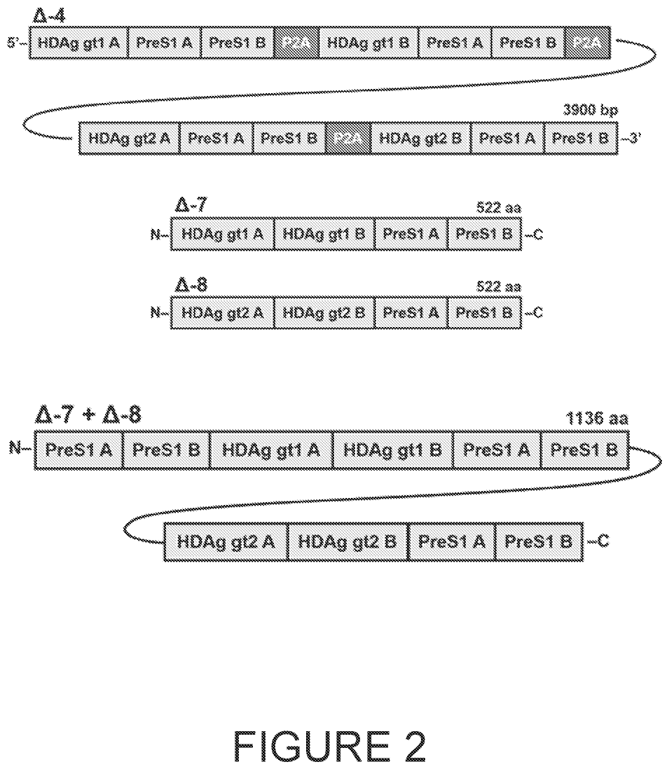

The use of recombinant HBV and HDV polypeptide constructs have been shown to be effective in eliciting antibody formation and immune protection against the two hepatitis viruses, for example, in WO 2017/132332, hereby expressly incorporated by reference in its entirety. These recombinant polypeptide constructs were assembled by combining HDAg chosen from four distinct HDV genotypes (HDAg genotype 1A, HDAg genotype 1 B, HDAg genotype 2 A, and HDAg genotype 2 B), PreS1 chosen from two genotype consensus sequences (PreS1 A and PreS1B), and one or more P2A autocatalytic peptide cleavage sites. Schematics for eleven recombinant constructs are shown in FIGS. 1A and 2, and corresponding SEQ ID NOs for DNA and polypeptide sequences, if applicable, are provided in Table 1. A Western blot confirms that polypeptides properly express from Δ-1 to Δ-10 recombinant constructs (FIG. 1B).

TABLE 1
SEQ ID NOs for the HBV/HDV immunogenic constructs

| | Human codon optimized | |
|---|---|---|
| Name | DNA Sequence | Polypeptide Sequence |
| Δ-1 (Delta-1; D1) | SEQ ID NO: 15 | SEQ ID NO: 25 |
| Δ-2 (Delta-2; D2) | SEQ ID NO: 16 | SEQ ID NO: 26 |
| Δ-3 (Delta-3; D3) | SEQ ID NO: 17 | SEQ ID NO: 07 |
| Δ-4 (Delta-4; D4) | SEQ ID NO: 18 | SEQ ID NO: 28 |
| Δ-5 (Delta-5; D5) | SEQ ID NO: 19 | SEQ ID NO: 29 |
| Δ-6 (Delta-6; D6) | SEQ ID NO: 20 | SEQ ID NO: 30 |
| Δ-7 (Delta-7; D7) | SEQ ID NO: 21 | SEQ ID NO: 31 |
| Δ-8 (Delta-8; D8) | SEQ ID NO: 22 | SEQ ID NO: 32 |
| Δ-9 (Delta-9; D9) | SEQ ID NO: 23 | SEQ ID NO: 33 |
| Δ-10 (Delta-10; D10) | SEQ ID NO: 24 | SEQ ID NO: 34 |
| Δ-7 + Δ-8 fusion | SEQ ID NO: 36 | SEQ ID NO: 37 |
| (Delta-7 + Delta-8; | (Wild type sequence: | |
| D-7 + D8) | SEQ ID NO: 35) | |

### Example 3: HBV/HDV DNA Compositions Induce Immunogenic Response in Mice

While immunogenic compositions and vaccines have traditionally been either whole organisms or antigenic proteins, it has been recently shown that in vivo administration of DNA to living tissue and the subsequent transcription and translation of antigenic proteins are also highly effective in triggering an immune response. These DNA immunogenic compositions are being explored as potential vaccine candidates against various diseases.

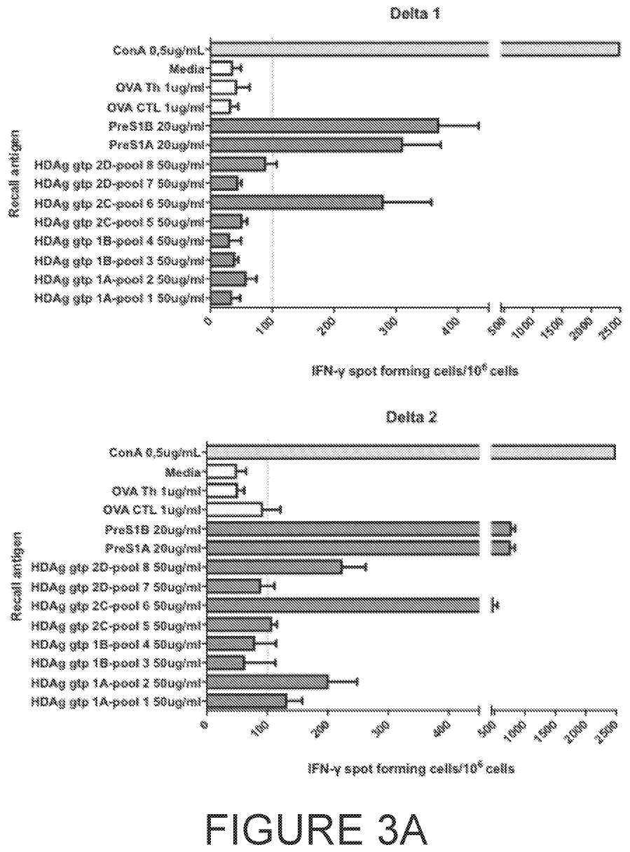

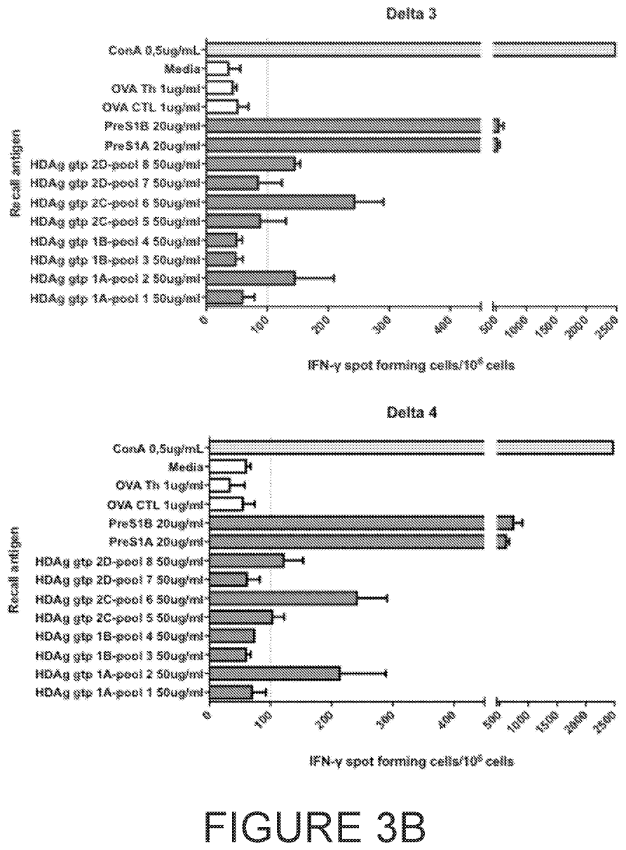

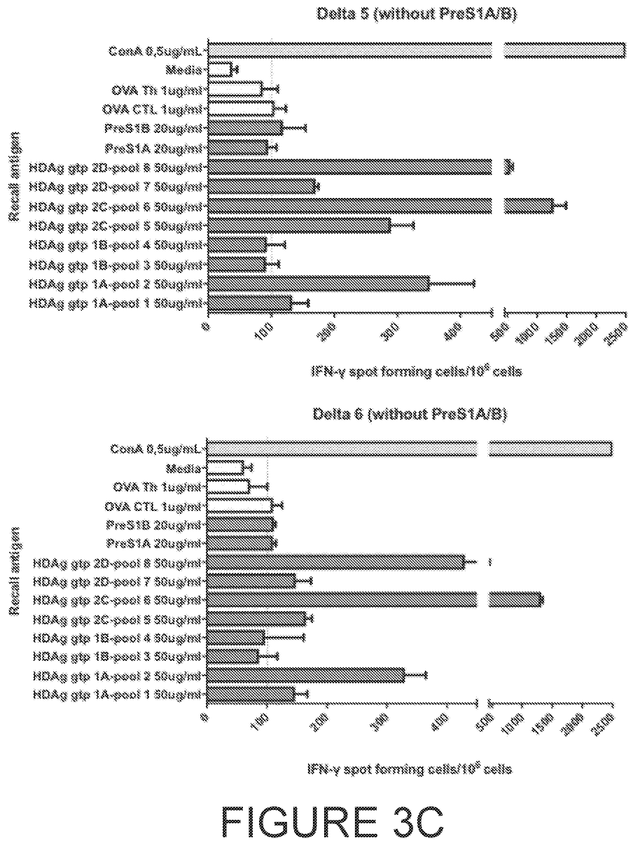

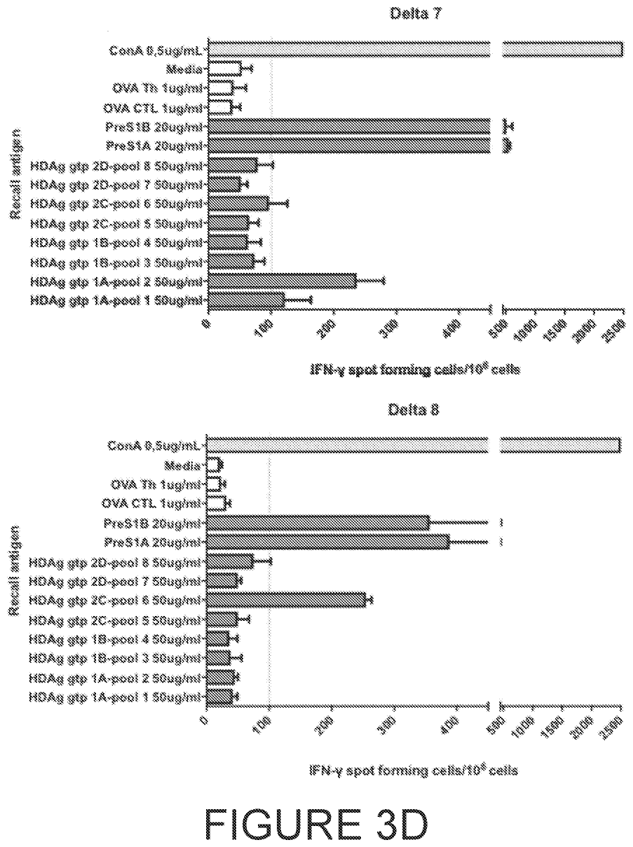

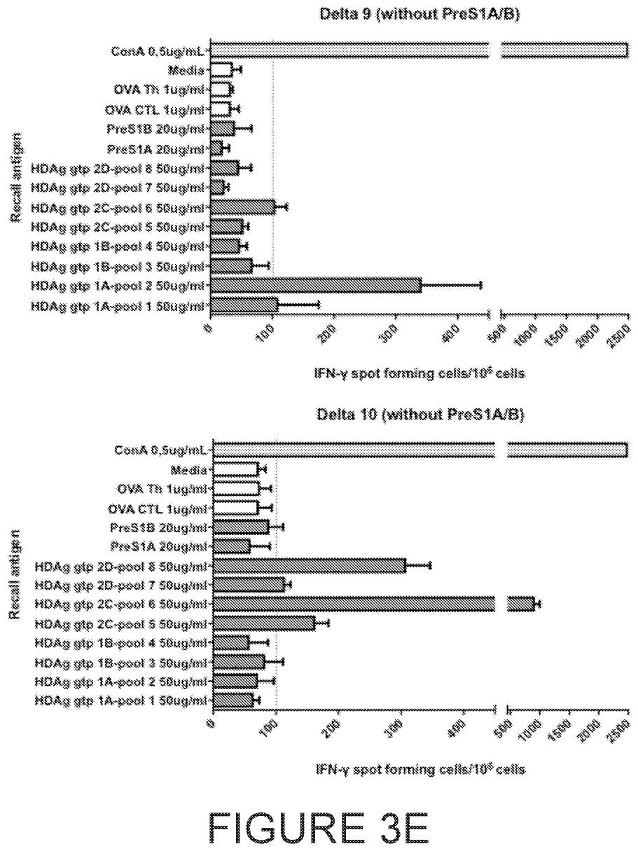

Following 2 weeks after the second administration of the DNA construct compositions, immunity of the mice against HBV and HDV antigens were assessed. White blood cells were purified from mouse whole blood samples and incubated with purified polypeptide antigens, including PreS1 A, PreS1 B, HDAg genotypes 1 A, 1 B, 2 A, and 2 B. Cells were also incubated with Concanavalin A (“ConA”) as a positive control, and two ovalbumin peptides (“OVA Th” and “OVA CTL”) as negative controls. The population frequency of interferon gamma (IFNγ) producing cells in response to antigen exposure was assessed by enzyme-linked immunospot assay (ELISpot). Briefly, white blood cells were incubated with antigen in wells coated with IFNγ antibodies. The cells were then removed, and biotinylated IFNγ antibodies, alkaline phosphatase-crosslinked streptavidin, and alkaline phosphatase substrate colorimetric reagents were added to the wells in succession with thorough washing in between. The plate was then allowed to dry and the remaining colored spots that correspond to IFNγ-secreting cells were counted by microscopy. The quantitative total of IFNγ spot forming cells per 10⁶ total cells in response to the various peptide antigens for each of the mice are shown in FIG. 3A (Δ-1 and Δ-2), 3B (Δ-3 and Δ-4), 3C (Δ-5 and Δ-6), 3D (Δ-7 and Δ-8), and 3E (Δ-9 and Δ-10).

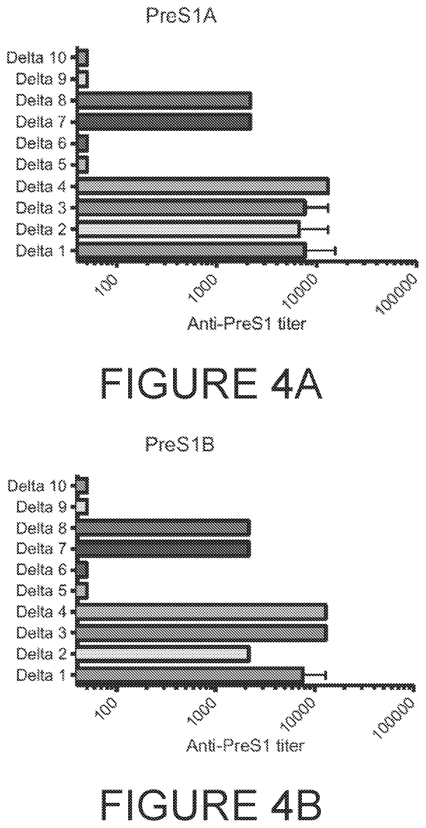

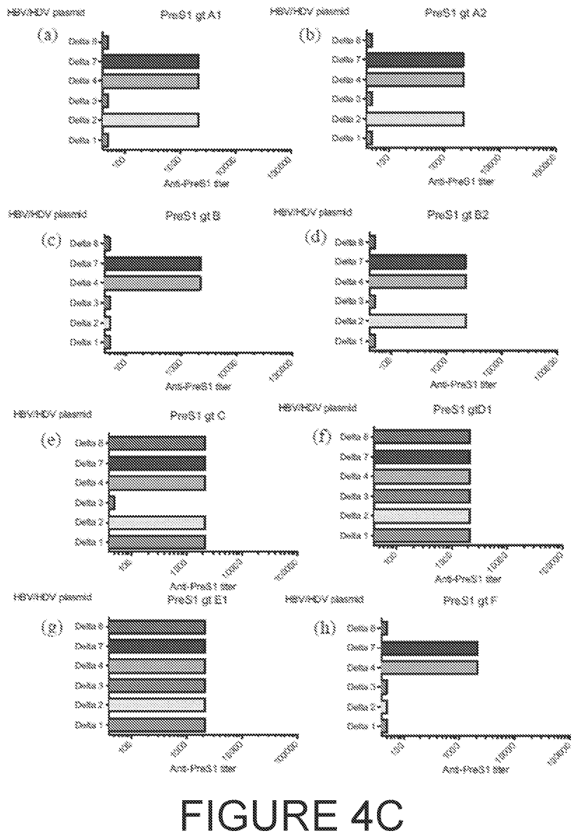

Anti-sera were tested for reactivity against PreS1A and PreS1B consensus peptides (aa 2-48) and for cross-reactivity against HBV (sub-) types A1, A2, B, B2, C, D1, E1 and F using pools of 20-mer PreS1-peptides. Immunogenic compositions comprising Δ-1, Δ-2, Δ-3, Δ-4, Δ-7, and Δ-8 resulted in robust immunogenicity against both HBV PreS1 antigens (FIGS. 4A-B). Δ-3 and Δ-4 induced antibody titers >10⁴ in mice, followed by constructs Δ-1, Δ-2, Δ-7 and Δ-8. Importantly, anti-sera from Δ-4 and Δ-7 immunized mice effectively cross-reacted between all tested HBV types (FIG. 4C). The immune response to HDAg peptides were more variable, likely due to differences in genotypic sequences, but typically greater than the ovalbumin controls. Notably, in Δ-3 and Δ-4 treated groups, a slight reduction in HDV T cell responses is observed when i.e, compared to constructs that contain only HDAg (Δ-5, Δ-6, Δ-9, Δ-10) which might be attributed to epitope recognition competition with the simultaneous priming of PreS1-specific T cells. Overall, this shows that active immunization is able to induce functional T cells to PreS1 and HDAg antigens and suggests that a broadly functional immunotherapy should contain both HDV genotypes 1 and 2 to ensure induction of specific T cells.

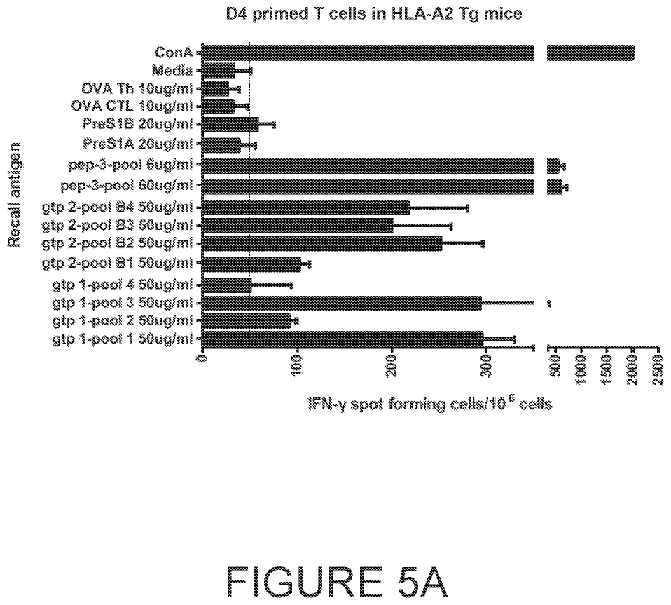

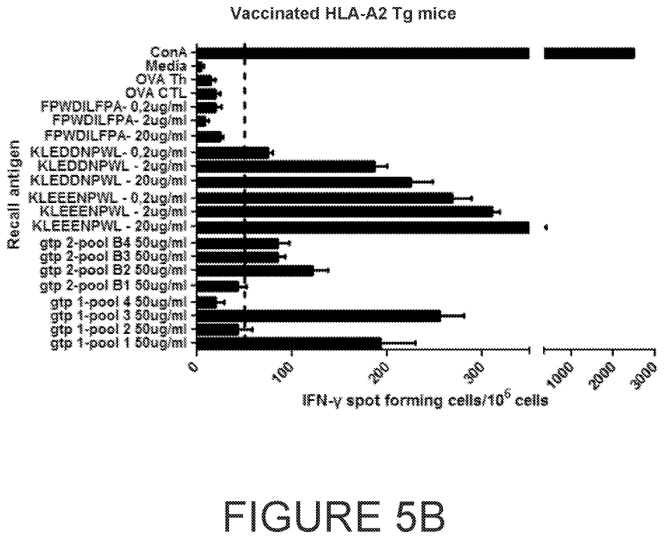

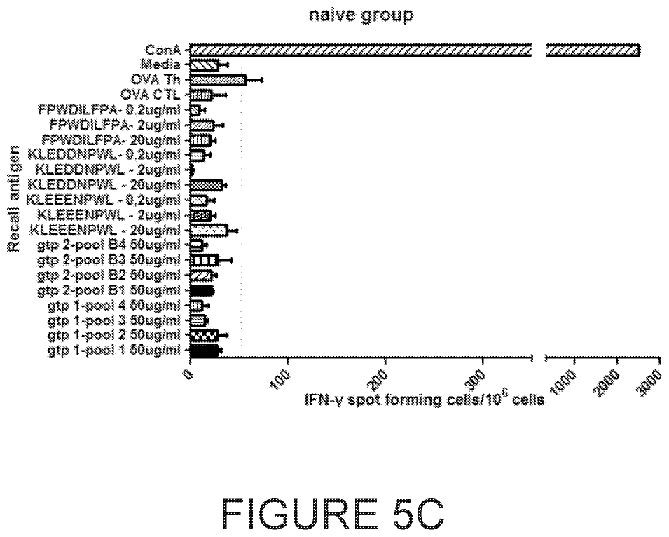

Similar experiments were performed with HLA-A2 restricted T cells purified from HLA-A2 transgenic HHD mice. IFNγ ELISpot of normal C57BL/6 (FIG. 5A) and HLA-A2 HHD (FIG. 5B) mice electroporated with a composition comprising Δ-4, along with naïve ULA-A2 HHD mice as control (FIG. 5C), confirms immunogenicity in the transgenic mice, suggesting efficacy of the DNA compositions for treating humans.

### Example 4: HBV/HDV DNA Compositions Induce Immunogenic Response in Rabbits

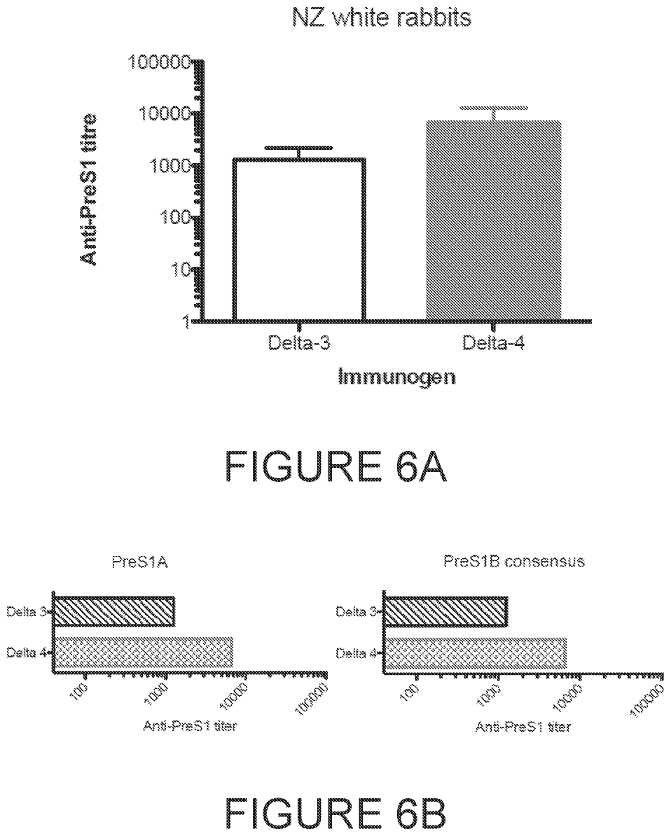

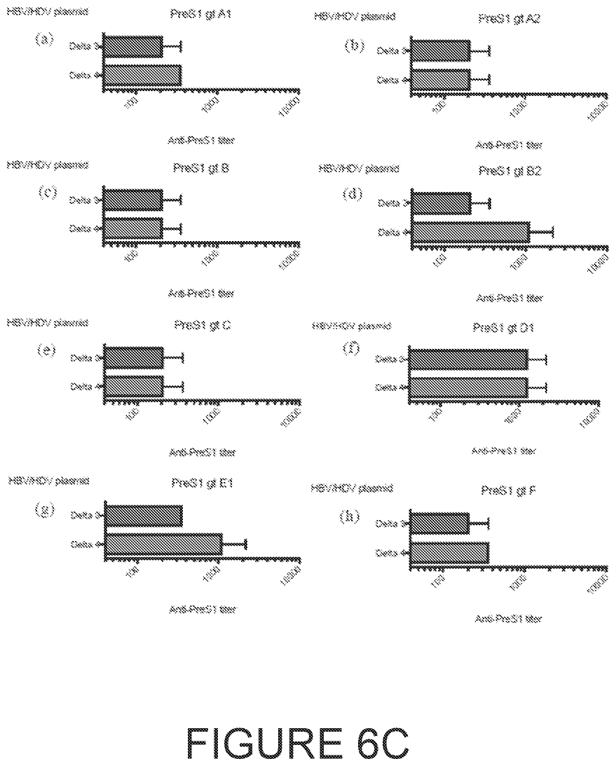

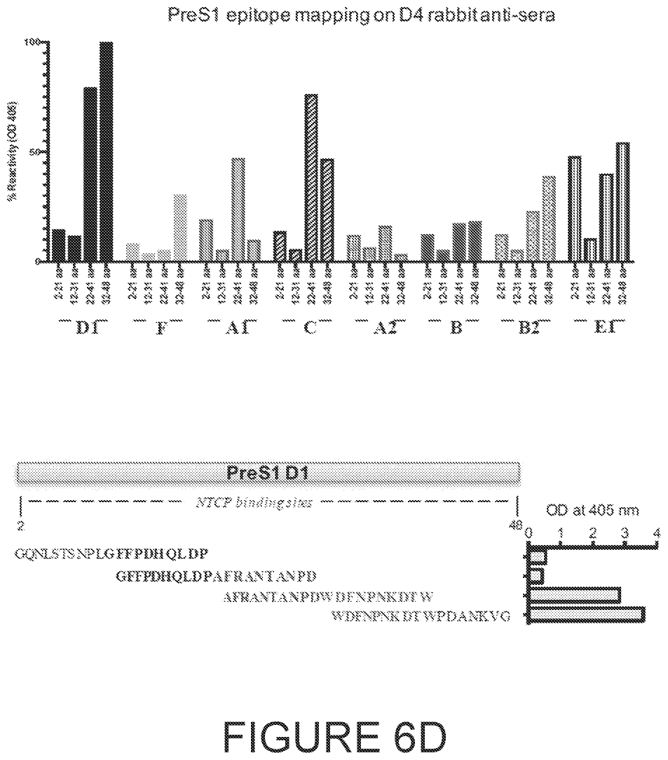

Corresponding experiments described in Example 3 were also performed in rabbits (Oryctolagus cuniculus). New Zealand white rabbits were injected intramuscularly with a saline solution containing 900 μg of DNA compositions comprising either Δ-3 or Δ-4 and subjected to electroporation. Doses were administered at 0 and 4 weeks. After immunization, anti-PreS1 antibody titers in the rabbit sera were observed for both DNA compositions comprising Δ-3 or Δ-4, with Δ-4 being more effective (>10³) (FIGS. 6A-B). Cross-reactivity of the rabbit antisera against HBV (sub-) types A1, A2, B, B2, C, D1, E1 and F using pools of 20-mer PreS1 peptides was also tested (FIG. 6C). The fine specificity of the rabbit D4 anti-sera was determined using individually 20-mer PreS1 peptides of HEW types A1, A2, B, B2, C, D1, E1 and F (FIG. 6D). This mapped the epitopes to PreS1 located to region 22-48 aa of genotype D1, as indicated by the higher reactivity, followed by lower reactivity to genotypes C, E1 and A1. This overlaps with the NTCP binding site and partly with previously identified epitopes recognized by neutralizing antibodies.

Table 2 summarizes the immunity effects of the ten DNA immunogenic compositions. DNA compositions comprising Δ-4 resulted in the greatest titer of anti-PreS1/anti-HBV antibodies in both mice and rabbits and are used in the prime/boost immunizations of the subsequent Examples. Δ-4 also shows the broadest reactivity to the different HMI genotypes. “n.d” denotes low or undetectable levels of antibody activity, “n/a” denotes that the experiment was not performed.

TABLE 2
HBV/HDV DMA vaccine screen (50 μg DNA im/EP)

| | HDV | Anti-PreS1 | Genotype cross- | Anti-PreS1 |
|---|---|---|---|---|
| Candidate | genotype (Gt) | titer in mice | reactivity in mice | titer in rabbits |
| Δ-1 | Gt 1 and 2 | 1:2160 | C, D, E | n/a |
| Δ-2 | Gt 1 and 2 | 1:2160 | A1, A2, B2, C, | <1:60 |
| | | | D, E | |
| Δ-3 | Gt 1 and 2 | 1:2160- | D, E | 1:360 |
| | | 1:12960 | | |
| Δ-4 | Gt 1 and 2 | 1:12960 | A-F | 1:2560 |
| Δ-5 | Gt 1 and 2 | n.d. | n.d. | n/a |
| Δ-6 | Gt 1 and 2 | n.d. | n.d. | n/a |
| Δ-7 | Gt 1 | 1:2160 | A-E | n/a |
| Δ-8 | Gt 2 | 1:2160 | C, D, E | n/a |
| Δ-9 | Gt 1 | n.d. | n.d. | n/a |
| Δ-10 | Gt 2 | n.d. | n.d. | n/a |

### Example 5: DNA Prime/Protein Boost Approach with HBV/HDV Constructs Improves Immunogenic Response in Mice

DNA compositions comprising Δ-4 (SEQ ID NO: 18) and polypeptide compositions comprising Δ-7 (SEQ ID NO: 31) or Δ-8 (SEQ ID NO: 32) were used for a DNA prime/protein boost immunization approach to build adaptive immunity and induce antibody production against HBV and/or HDV in vivo (FIG. 2).

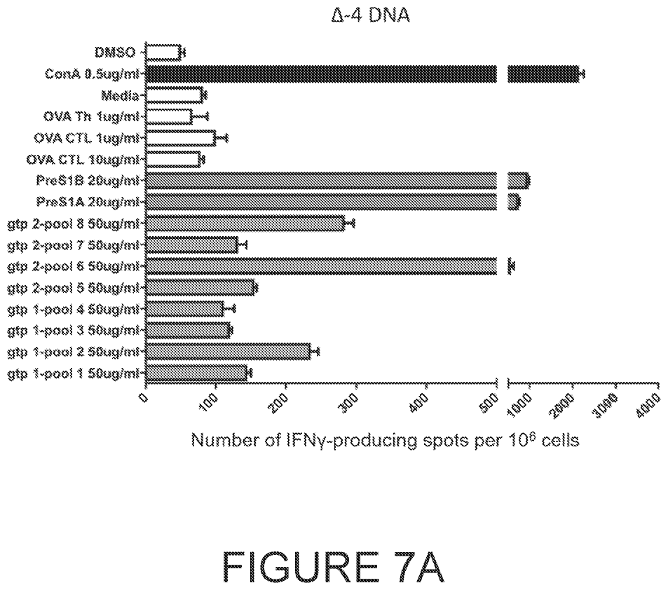

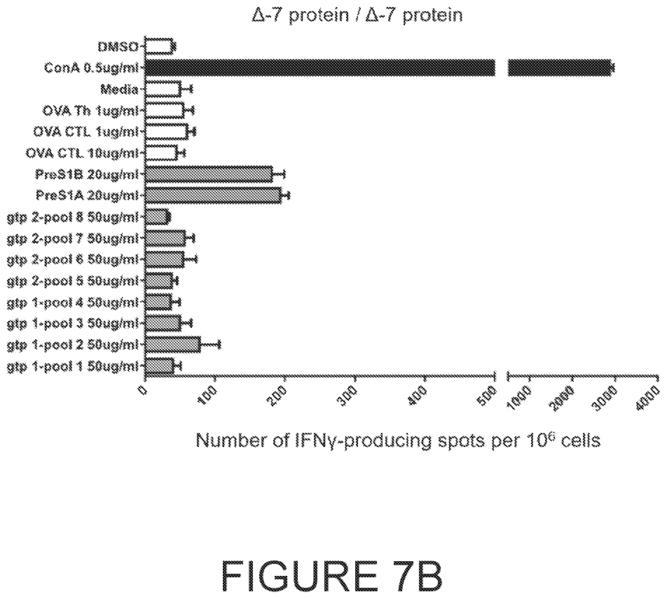

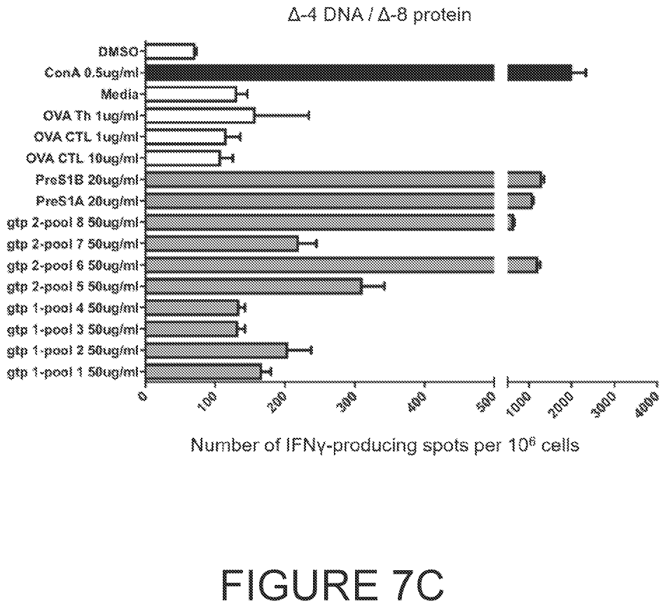

C57BL/6 mice were immunized with (1) a DNA composition comprising Δ-4 (3 sequential doses of 50 μg DNA), (2) a polypeptide composition comprising Δ-7 (3 sequential does of 20 μg protein with alum adjuvant), or (3) a DNA composition comprising Δ-4 followed by a polypeptide composition comprising Δ-8 (2 doses of 50 μg DNA then 2 doses of 20 μg protein with alum). After administration of the compounds, purified white blood cells were tested for IFNγ production in response to HBV and HDV antigens by ELISpot (as described in Examples 1 and 2). Mice treated with (1) exhibited a commensurate response to hepatitis antigens (FIG. 7A) observed in Example 3 and FIG. 3B, but mice treated with the DNA prime/protein boost compositions of (3) resulted in a comparatively stronger immune cell response overall (FIG. 7C). As Δ-8 includes sequences for the HDAg genotype 2 polypeptides, the assayed immune response is particularly improved against these antigens (FIG. 7C, gtp 2-pool 5, 6, 7 and 8). Conversely, the protein-only approach of (2) using Δ-7 polypeptides fails to both elicit an equally effective immune response for both HBV and HDV antigens (FIG. 7B). This demonstrates that this DNA prime/protein boost approach may be effective at inducing a robust immunogenic response greater than traditional protein or organism-based compositions for certain pathogens, including HBV and HDV.

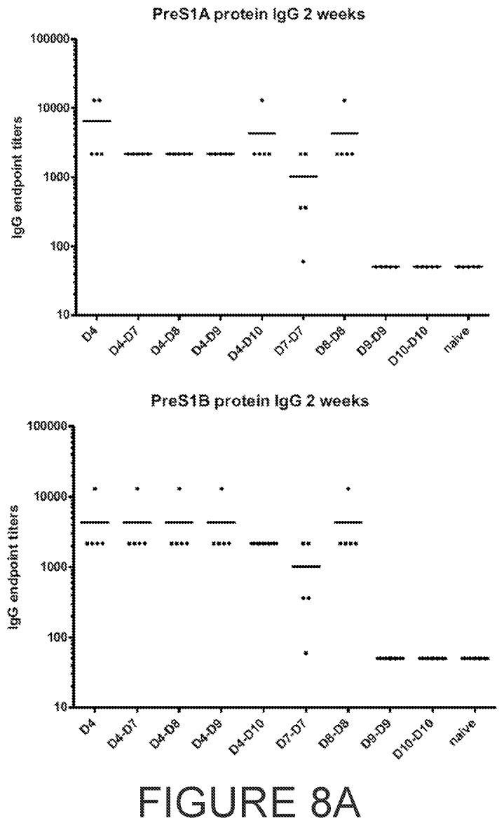

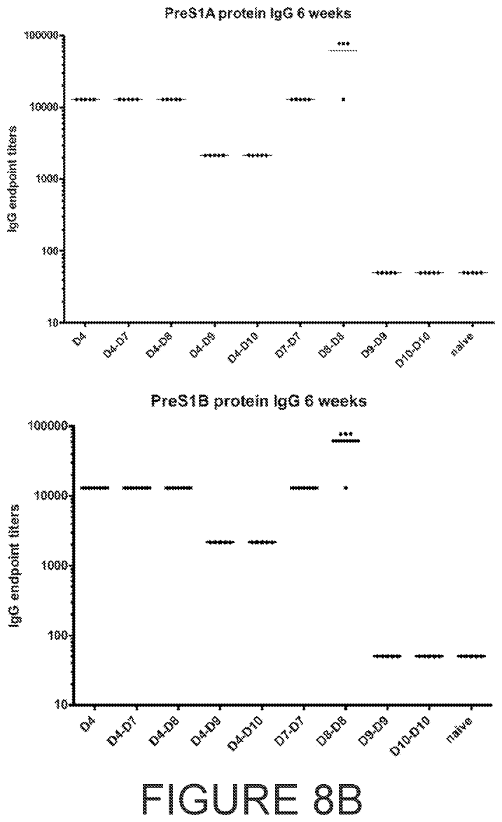

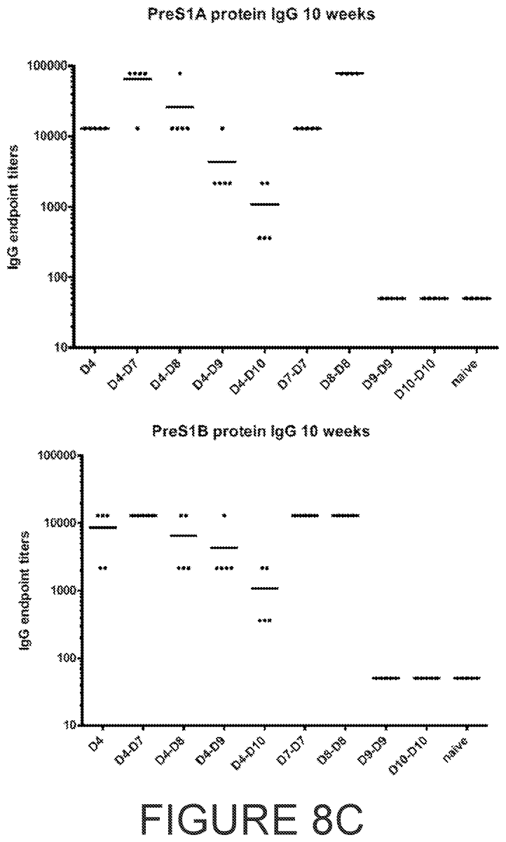

Other DNA prime/protein boost combinations were also assessed in mice. Anti-PreS1 IgG titers in mice were measured after immunization with (1) a DNA-only composition comprising Δ-4 (“D4”), (2) protein-only compositions comprising Δ-7 (“D7-D7”), Δ-8 (“D8-D8”), Δ-9 (“D9-D9”), or Δ-10 (“D10-D10”), or (3) DNA-protein compositions comprising Δ-4 DNA with Δ-7 (“D4-D7”), Δ-8 (“D4-D8”), Δ-9 (“D4-D9”), or Δ-10 (“D4-D10”) protein. Compositions were administered three times at weeks 0, 4, and 8, with either 50 μg DNA inn/EP or 20 μg protein with alum administered for each dose. For DNA-protein compositions (3), 50 μg DNA im/EP was administered for the first dose at week 0, and 20 μg protein with alum was administered for the second and third doses at weeks 4 and 8. Anti-PreS1 IgG titers in sera were assessed after 2 weeks (FIG. 8A), 6 weeks (FIG. 8B), and 10 weeks (FIG. 8C) after the first dosage (i.e. 2 weeks after each dosage). DNA prime/protein boost composition D4-D7 results in superior anti-PreS1 titers after the completion of the dose administration schedule.

### Example 6: DNA Prime/Protein Boost Approach with HBV/HDV Constructs Improves Immunogenic Response in Rabbits

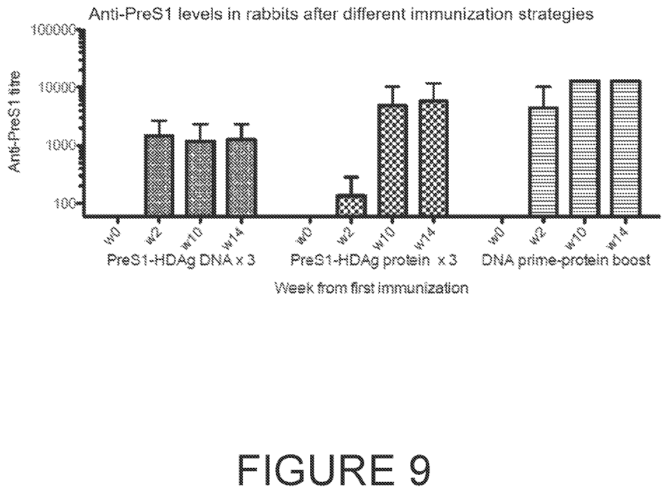

New Zealand white rabbits were immunized with (1) a DNA-only composition comprising Δ-4, (2) a protein-only composition comprising Δ-4, or (3) a DNA prime/protein boost composition comprising Δ-4 DNA and Δ-4 protein. Compositions were administered four times as weeks 0, 4, 8, and 12, with either 900 μg DNA im/EP or 300 μg protein with alum administered for each dose. For DNA-protein compositions (3), 900 μg DNA im/EP was administered for the first dose at week 0, and 300 μg protein with alum was administered for the second, third, and fourth doses at weeks 4, 8, and 12. Anti-PreS1 IgG titers in sera were assessed at weeks 0, 2, 10, and 14 (i.e. 2 weeks after each dosage) (FIG. 9). Not only does the DNA prime/protein boost composition (3) result in greater overall titers compared to DNA-only (1) and protein-only (2) compositions, but also induces robust antibody production more rapidly, by week 2, relative to the protein-only composition.

### Example 7: Adoptive Transfer of Sera or Purified IgG from Immunized Animals Protects Humanized Mice Against HBV and HMI Challenges

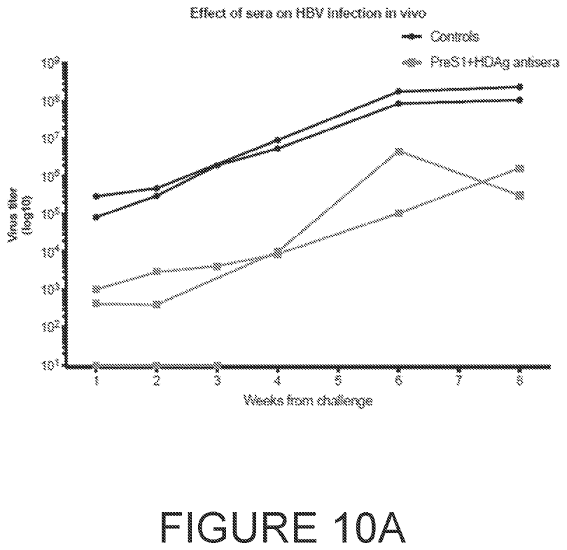

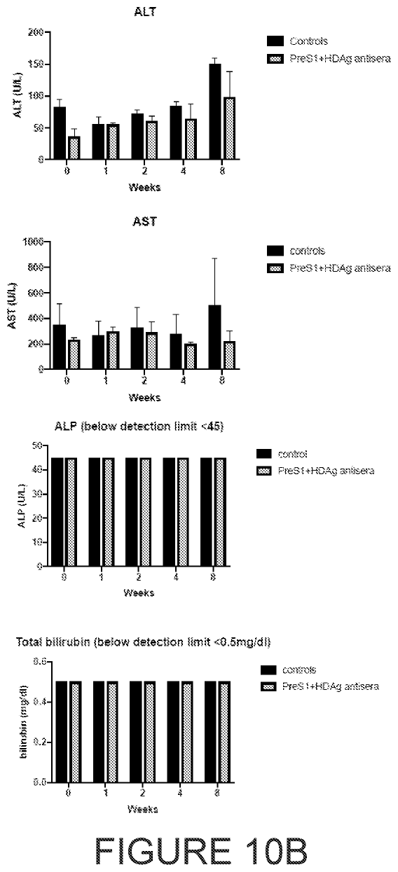

The ability of D4 induced antibodies to neutralize HBV infection in vivo was determined using human-liver chimeric uPA^{+/+}-SCID mouse model as described. Total IgG was purified from D4-immunized and non-immunized rabbits and were injected in uPA^{+/+}-SCID mice repopulated with human hepatocytes three days prior to HBV challenge. The D4-induced PreS1 IgG antibodies protected, or significantly delayed peak viremia in all challenged mice (FIG. 10A). Out of three challenged mice, one was protected (weeks 1 to 3) whereas the other two developed serum levels of HBV<10⁴ IU/ml up to a monthly screening and remained lower compared to the controls up to 7 weeks follow up. The control mice treated with IgG from a naïve rabbit all reached serum HBV DNA levels exceeding 10⁸ IU/ml. There were no significant differences between the groups with respect to serum levels of alanine transferase, asparagine transferase, alkaline phosphatase, or bilirubin (FIG. 10B). In conclusion, passive immunization with D4-specific PreS1 IgG antibodies given as a single dose was able to prevent, or significantly delayed HBV infection in vivo in mice repopulated with human hepatocytes (Table 3). Importantly, the inoculum contains high levels of sub-viral particles SHBsAg showing that these antibodies indeed escape being blocked by SHBsAg. The PreS1 antibodies present at inoculation and during the first weeks clearly block infection, or the first rounds of infection, and limit the number of infected hepatocytes. This limits the viral spread and delay development of peak viremia.

TABLE 3
Adoptive transfer from DNA/protein vaccinated animals protects against HBV/HDV

| | Transfer | | Number | Absence of | Low level | High level |
|---|---|---|---|---|---|---|
| Infection | source | Treatment | of mice | infection | infection | infection |
| HBV | Purified | Control | 3 | 0 | 0 | 3 |
| | rabbit IgG | | | | | |
| HBV | Purified | D4 DNA | 3 | 1 | 2 | 0 |
| | rabbit IgG | | | | | |
| HBV/HDV | Whole | Control | 3 | 0 | 0 | 3 |
| | mouse sera | | | | | |
| HBV/HDV | Whole | D7 Protein/alum | 2 | 2 | 0 | 0 |
| | mouse sera | | | | | |
| HBV/HDV | Whole | D4 DNA +D7/alum | 3 | 3 | 0 | 0 |
| | mouse sera | | | | | |

### Example 8: Challenge of HBV/HDV Peptide Constructs with Different Adjuvants

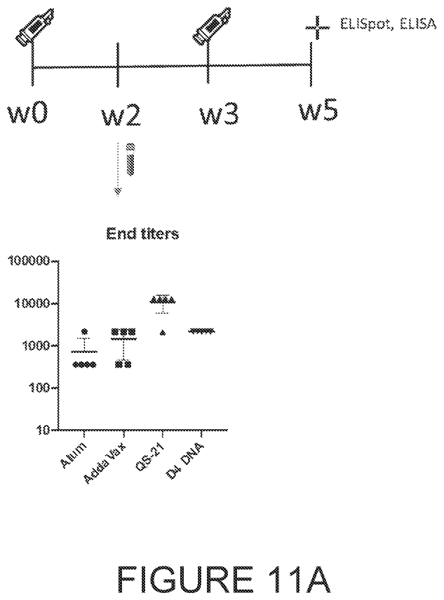

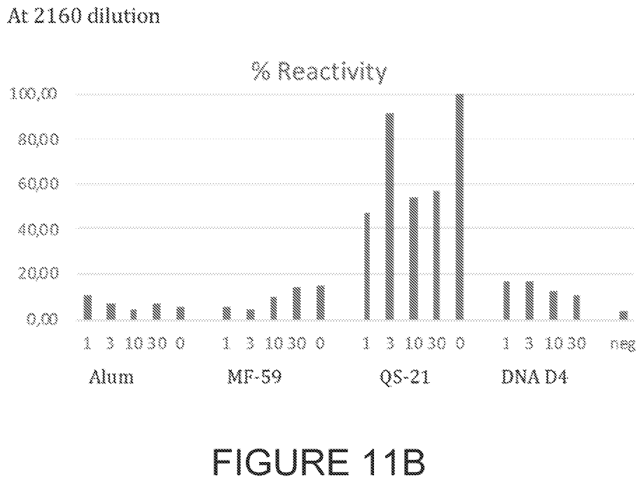

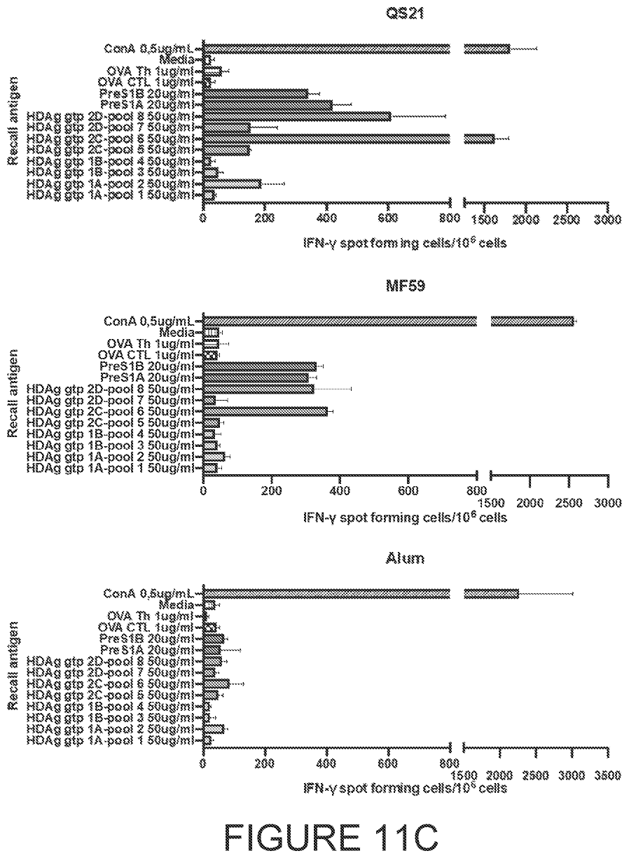

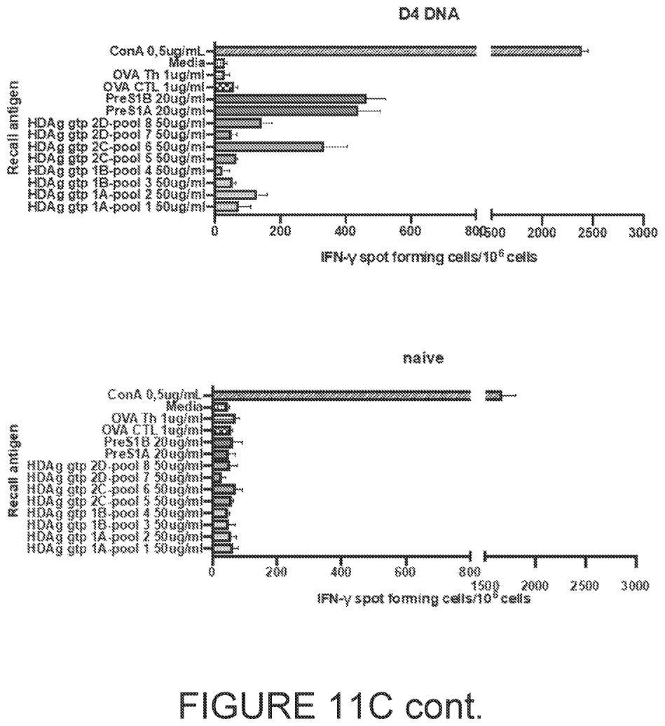

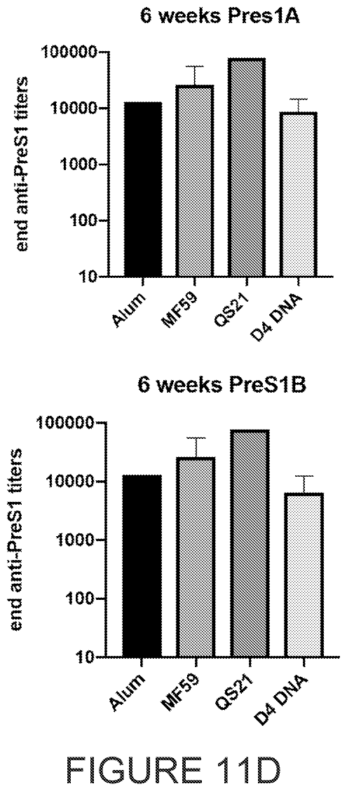

Mixtures of D-7 and D-8 peptides were assessed using different adjuvants. C57BL/6J mice were administered with 2 rounds of a 20 μg mixture of D-7 and D-8 peptides (10 μg each of D-7 and D-8) administered at week 0 and week 3 (FIG. 11A). Peripheral blood samples were taken at week 2 (between the two rounds) to determine end titers for HBV and HDV reactivity by ELISA (FIGS. 11A and 11B), and splenocytes were isolated for HBV and HDV reactivity by ELISpot (FIG. 11C-D) at week 5. Peptide compositions were administered subcutaneously with QS-21, MF59, and alum adjuvants. Naïve mice and mice administered D-4 DNA plasmid by intramuscular electroporation were used as controls. IFNγ ELISpot was performed as described above using pools of HDAg peptides, PreS1A and PreS1B peptides, with OVA peptides and Concanavalin A as controls (FIGS. 11C-D). Compositions administered with QS-21 adjuvant exhibited elevated HDAg reactivity compared to the other adjuvants. 5 mice per group were tested.

### Example 9: Comparison of Exemplary HBV/HDV DNA and/or Peptide Constructs

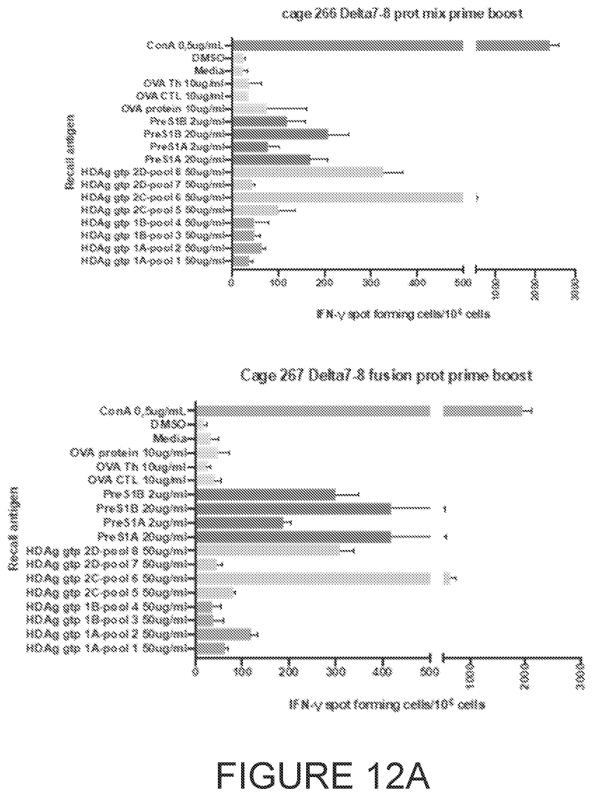

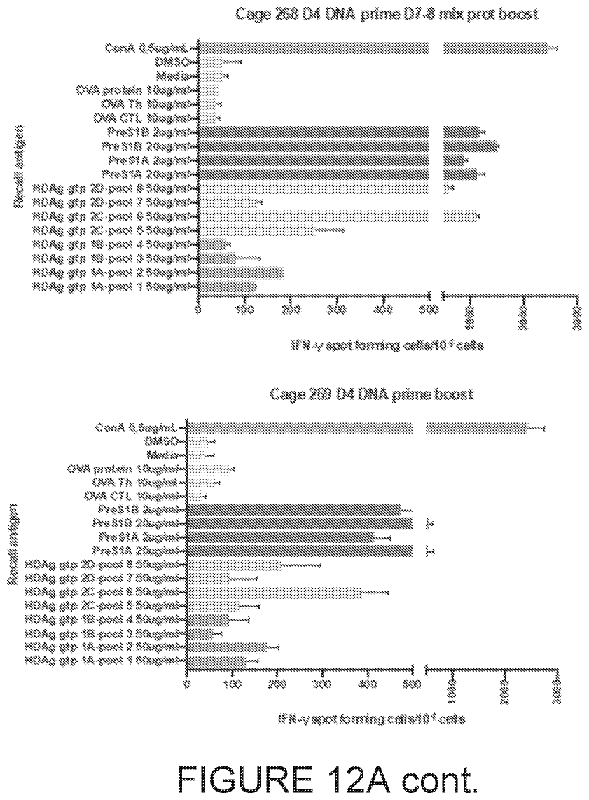

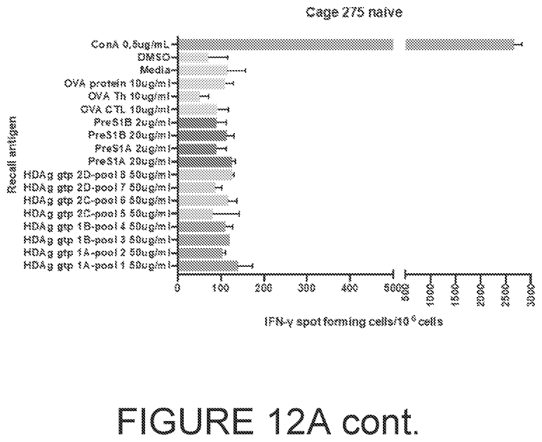

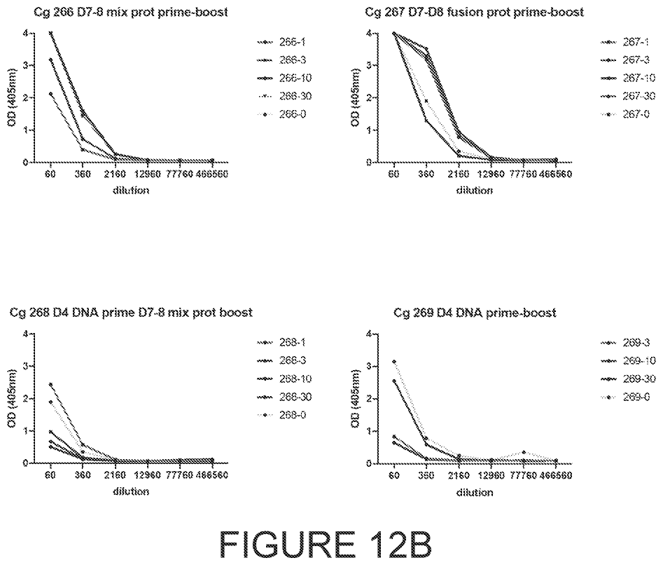

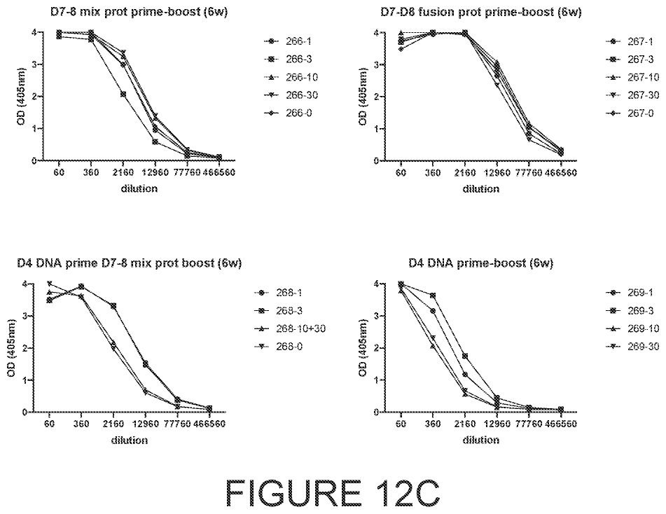

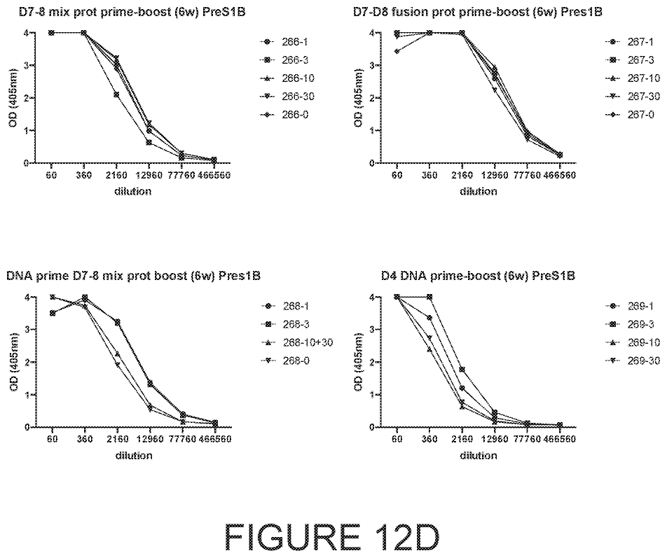

A comparison of immunogenicity for 1) a mixture of D-7 and D-8 peptide only, 2) D-7+D-8 fusion peptide only, 3) D-4 DNA prime and a mixture of D-7 and D-8 peptide boost were tested, with D-4 DNA only and naïve conditions as controls. Mice were administered with either 20 μg of the D-7+D-8 fusion protein or 10 μg each of D-7 and D-8 peptide in the mixed conditions with QS-21 adjuvant, subcutaneously at the tail base in a volume of 100 μL. 2 rounds of administration was performed at weeks 0 and 4. D-4 DNA control was administered at 50 μg intramuscular in 50 μL PBS with electroporation. At week 6 (following two rounds of administration), T cell response to PreS1 and HDV antigen genotypes 1 and 2 were determined by IFNγ ELISpot (FIG. 12A). In addition, at weeks 2 (following one round of administration) and 6 weeks (following two rounds of administration), antibody levels to PreS1A (FIG. 12B-C) and PreS1B (FIG. 12D) consensus peptides were assessed. The greatest HBV and HDV reactivity are observed in the DNA prime, peptide boost condition.

### Example 10: DNA or Protein Prime with DNA or Protein Boost Immunization Against HBV and/or HDV in Human Clinical Trials

The following example describes embodiments of using an immunogenic composition or product combination, optionally comprised of a nucleic acid component and a polypeptide component, used to treat or prevent viral infections caused by viruses such as HBV and HDV.

The DNA prime/protein boost compositions as described in Example 5 are administered to human patients enterally, orally, intranasally, parenterally, subcutaneously, intramuscularly, intradermally, or intravenously. These human patients may be currently infected with HBV and/or HDV, previously infected with HBV and/or HDV, at risk of being infected with HBV and/or HDV, or uninfected with HBV and/or HDV.

The DNA prime doses are administered first, at an amount of 1, 10, 100, 1000 ng, or 1, 10, 50, 100, 200, 300, 400, 500, 600, 700, 800, 900, 1000 μg, or 1, 10, 100, 200, 300, 400, 500, 600, 700, 800, 900, 1000 mg, or any amount within a range defined by any two of the aforementioned amounts, or any other amount appropriate for optimal efficacy in humans. After the first DNA prime dose, 1, 2, 3, 4, or 5 additional DNA prime doses can be administered 1, 2, 3, 4, 5, 6, 7, 8, 9, 10, 11, 12, 24, 36, or 48 days or weeks or any time within a range defined by any two of the aforementioned times after administration of the previous DNA prime dose, e.g., within 1-48 days or 1-48 weeks. The protein boost doses are administered following the DNA prime doses, at an amount of 1, 10, 100, 1000 ng, or 1, 10, 50, 100, 200, 300, 400, 500, 600, 700, 800, 900, 1000 μg, or 1, 10, 100, 200, 300, 400, 500, 600, 700, 800, 900, 1000 mg, or any amount within a range defined by any two of the aforementioned amounts, or any other amount appropriate for optimal efficacy in humans. The first protein boost dose is administered 1, 2, 3, 4, 5, 6, 7, 8, 9, 10, 11, 12, 24, 36, or 48 days or weeks or any time within a range defined by any two of the aforementioned times after administration of the final DNA prime dose. After the first protein boost dose, 1, 2, 3, 4, or 5 additional protein boost doses can be administered 1, 2, 3, 4, 5, 6, 7, 8, 9, 10, 11, 12, 24, 36, or 48 days or weeks or any time within a range defined by any two of the aforementioned times after administration of the previous protein boost dose.

Patients will be monitored for successful response against HBV and/or HDV, for example, production of anti-HBV, anti-HDV, anti-PreS1, or anti-HDAg antibodies in sera, rapid activation of T cells and other immune cells when exposed to HBV and/or HDV antigens, and protection against future HBV and/or HDV infections.

In patients currently infected, previously infected, or at risk for infection with HBV and/or HDV, administration of the DNA prime/protein boost compositions may be performed in conjunction with antiviral therapy. Potential antiviral therapy therapeutics that have been shown to be effective against HBV or HDV include but are not limited to entecavir, tenofovir, lamivudine adefovir, telbivudine, emtricitabine, interferon-α, pegylated interferon-α, or interferon alfa-2b, or any combination thereof. Patients will be monitored for side effects such as dizziness, nausea, diarrhea, depression, insomnia, headaches, itching, rashes, fevers, or other known side effects of the provided antiviral therapeutics.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

### REFERENCES

Razavi-Shearer D, Gamkrelidze I, Nguyen M H, Chen D-S, Van Damme P, Abbas Z, et al. Global prevalence, treatment, and prevention of hepatitis B virus infection in 2016: a modeling study. Lancet Gastroenterol Hepatol 2018; 3:383-403.

Trépo C, Chan H L Y, Lok A. Hepatitis B virus infection. Lancet 2014; 384:2053-2063.

WHO|Global hepatitis report, 2017. WHO 2018.

Mitra B, Thapa R J, Guo H, Block T M. Host functions used by hepatitis B virus to complete its life cycle: Implications for developing host-targeting agents to treat chronic hepatitis B. Antiviral Res 2018; 158:185-198.

Chen H-Y, Shen D-T, Ji D-Z, Han P-C, Zhang W-M, Ma J-F, et al. Prevalence and burden of hepatitis D virus infection in the global population: a systematic review and meta-analysis. Gut 2018; :gutjnl-2018-316601.

Liu J, Li T, Zhang L, Xu A. The Role of Hepatitis B Surface Antigen in Nucleos(t)ide Analogues Cessation among Asian Chronic Hepatitis B Patients: A Systematic Review. Hepatology Published Online First: 18 Dec. 2018. doi:10.1002/hep.30474

Nassal M. HBV cccDNA: viral persistence reservoir and key obstacle for a cure of chronic hepatitis B. Gut 2015; 64:1972-1984.

Papatheodoridis G V., Manolakopoulos S, Touloumi G, Vourli G, Raptopoulou-Gigi M, Vafiadis-Zoumbouli I, et al. Virological suppression does not prevent the development of hepatocellular carcinoma in HBeAg-negative chronic hepatitis B patients with cirrhosis receiving oral antiviral(s) starting with lamivudine monotherapy: results of the nationwide HEPNET. Greece cohort study. Gut 2011; 60:1109-1116.

Zoulim F, Mason W S. Reasons to consider earlier treatment of chronic HBV infections. Gut 2012; 61:333-336.

Heidrich B, Yurdaydin C, Kabaçam G, Ratsch B A, Zachou K, Bremer B, et al. Late HDV RNA relapse after peginterferon alpha-based therapy of chronic hepatitis delta. Hepatology 2014; 60:87-97.

Wedemeyer H, Yurdaydin C, Dalekos G N, Erhardt A, C̨akalo\check{g}lu Y, De\check{g}ertekin H, et al. Peginterferon plus Adefovir versus Either Drug Alone for Hepatitis Delta. N Engl J Med 2011; 364:322-331.

Feld J J, Terrault N A, Lin H S, Belle S H, Chung R T, Tsai N, et al. Entecavir and peginterferon alfa-2a in adults with HB eAg-positive immune tolerant chronic hepatitis B virus infection. Hepatology 2018; :hep.30417.

Chen M, Sällberg M, Thung S N, Hughes J, Jones J, Milich D R. Nondeletional T-cell receptor transgenic mice: model for the CD4(+) T-cell repertoire in chronic hepatitis B virus infection. J Virol 2000; 74:7587-99.

Chen M, Sallberg M, Hughes J, Jones J, Guidotti L G, Chisari F V., et al. Immune Tolerance Split between Hepatitis B Virus Precore and Core Proteins. J Virol 2005; 79:3016-3027.

Chen M T, Billaud J-N, Sallberg M, Guidotti L G, Chisari F V., Jones J, et al. A function of the hepatitis B virus precore protein is to regulate the immune response to the core antigen. Proc. Natl Acad Sci 2004; 101:14913-14918.

Mason W S, Gill U S, Litwin S, Zhou Y, Peri S, Pop O, et al. HBV DNA Integration and Clonal Hepatocyte Expansion in Chronic Hepatitis B Patients Considered Immune Tolerant. Gastroenterology 2016; 151:986-998.e4.

Milich D R. The Concept of Immune Tolerance in Chronic Hepatitis B Virus Infection Is Alive and Well. Gastroenterology 2016; 151:801-804.

Short J M Chen S, Roseman A M, Butler P J G, Crowther R A, Structure of Hepatitis B Surface Antigen from Subviral Tubes Determined by Electron Cryomicroscopy. J Mol Biol 2009; 390:135-141.

Rydell G E, Prakash K, Norder H, Lindh M. Hepatitis B surface antigen on subviral particles reduces the neutralizing effect of anti-HBs antibodies on hepatitis B viral particles in vitro. Virology 2017; 509:67-70.

Dryden K A, Wieland S F, Whitten-Bauer C, Gerin J L, Chisari F V., Yeager M. Native Hepatitis B Virions and Capsids Visualized by Electron Cryomicroscopy. Mol Cell 2006; 22:843-850.

Ni Y, Sonnabend J, Seitz S, Urban S. The Pre-S2 Domain of the Hepatitis B Virus Is Dispensable for Infectivity but Serves a Spacer Function for L-Protein-Connected Virus Assembly. J Virol 2010; 84:3879-3888.

Ni Y, Lempp F A, Mehrle S, Nkongolo S, Kaufman C, Faith M, et al. Hepatitis B and D Viruses Exploit Sodium Taurocholate Co-transporting Polypeptide for Species-Specific Entry into Hepatocytes. Gastroenterology 2014; 146:1070-1083.e6.

Chen A, Ahlen G, Brenndorfer E D, Brass A, Holmstrom F. Chen M, et al. Heterologous T Cells Can Help Restore Function in Dysfunctional Hepatitis C Virus Nonstructural 3/4A-Specific Cells during Therapeutic Vaccination. J Imunol 2011; 186:5107-5118.

Mancini-Bourgine M, Fontaine H, Scott-Algara D. Pol. S, Bréchot C, Michel M-L Induction or expansion of T-cell responses by a hepatitis B DNA vaccine administered to chronic HBV carriers. Hepatology 2004; 40:874-882.

Kosinska A D, Zhang E, Johrden L, Liu J, Seiz P L, Zhang X, et al. Combination of DNA Prime—Adenovirus Boost Immunization with Entecavir Elicits Sustained Control of Chronic Hepatitis B in the Woodchuck Model. PLoS Pathog 2013; 9:e1003391.

Brass A, Frelin L, Milich D R, Sällberg M, Ahlén G. Functional Aspects of Intrahepatic Hepatitis B Virus-specific T Cells Induced by Therapeutic DNA Vaccination. Mol Ther 2015; 23:578-590.

Yuen M F, Gane E J, Kim D J, Weilert F, Chan H L Y, Lalezari J, et al. Antiviral Activity, Safety, and Pharmacokinetics of Capsid Assembly Modulator NVR 3-778 in Patients with Chronic HBV Infection. Gastroenterology Published Online First: 6 Jan. 2019. doi:10.1053/J. GASTRO. 2018 Dec. 23

Liang T J, Block T M, McMahon B J, Ghany M G, Urban S, Guo J-T, et al. Present and future therapies of hepatitis B: From discovery to cure. Hepatology 2015; 62:1893-1908.

Meuleman P, Vanlandschoot P, Leroux-Roels G. A simple and rapid method to determine the zygosity of uPA-transgenic SCID mice. doi:10.1016/S0006-291X(03)01388-3

Meuleman P, Libbrecht L, De Vos R, de Hemptinne B, Gevaert K, Vandekerckhove J, et al. Morphological and biochemical characterization of a human liver in a uPA-SCID mouse chimera. Hepatology 2005; 41:847-856.

Levander S, Holmström F, Frelin L, Ahlén G, Rupp D, Long G, et al. Immune-mediated effects targeting hepatitis C virus in a syngeneic replicon cell transplantation mouse model. Gut 2018; 67:1525-1535.

Ahlén G, Söderholm J, Tjelle Kjeken R, Frelin L, Höglund et al. In vivo electroporation enhances the immunogenicity of hepatitis C virus nonstructural 3/4A DNA by increased local DNA uptake, protein expression, inflammation, and infiltration of CD3+ T cells. J Immunol 2007; 179:4741-53.

Ni Y, Urban S. Hepatitis B Virus Infection of HepaRG Cells, HepaRG-hNTCP Cells, and Primary Human Hepatocytes. In: Methods in molecular biology (Clifton, N.J.,); 2017. pp. 15-25.

Donkers J M, Zehnder B, van Western G J P, Kwakkenbos M J, IJzerman A P, Oude Elferink R P J, et al. Reduced hepatitis B and D viral entry using clinically applied drugs as novel inhibitors of the bile acid transporter NTCP. Sci Rep 2017; 7:15307.

Meulenian P, Lerouxroels G. The human liver-uPA-SCID mouse: A model for the evaluation of antiviral compounds against HBV and HCV. Antiviral Res 2008; 80:231-238.

Wi J, Jeong M S, Hong H J. Construction and characterization of an anti-hepatitis B virus preS1 humanized antibody that binds to the essential receptor binding site. J Microbiol Biotechnol 2017; 27:1336-1344.

Lok A S F, McMahon B J, Brown R S, Wong J B, Ahmed AT, Farah W, et al. Antiviral therapy for chronic hepatitis B viral infection in adults: A systematic review and meta-analysis, Hepatology 2016; 63:284-306.

Revill P A, Chisari V, Block J M, Dandri M, Gehring A J, Guo H, et al. A global scientific strategy to cure hepatitis B. Lancet Gastroenterol Hepatol Published Online First: 10 Apr. 2019. doi:10.1016/52468-1253(19)30119-0

Neurath A R, Kent S B, Strick N, Parker K. Identification and chemical synthesis of a host cell receptor binding site on hepatitis B virus. Cell 1986; 46:429-36.

Gripon P, Le Seyec J, Rumin S, Guguen-Guillouzo C. Myristylation of the Hepatitis B Virus Large Surface Protein Is Essential for Viral Infectivity. Virology 1995; 213:292-299.

Hong H J, Ryu C J, Hur H, Kim S, Oh H K, Oh M S, et al. In vivo neutralization of hepatitis B virus infection by an anti-preS1 humanized antibody in chimpanzees. Virology 2004; 318:134-141.

Bogomolov P, Alexandrov A, Voronkova N, Macievich M, Kokina K, Petrachenkova M, et al. Treatment of chronic hepatitis D with the entry inhibitor myrcludex B: First results of a phase Ib/IIa study. J Hepatol 2016; 65:490-498.

Blank A, Eidam A, Haag M, Hohmann N, Burhenne J, Schwab M, et al. The NTCP-inhibitor Myrcludex B: Effects on Bile Acid. Disposition and Tenofovir Pharmacokinetics. Clin Pharmacol Ther 2018; 103:341-348.

Passioura T, Watashi K, Fukano K, Sureau C, Suga H, Correspondence T W, De Novo Macrocyclic Peptide Inhibitors of Hepatitis B Virus Cellular Entry. Cell Chem Biol 2018; 25:906-915.

Bian Y, Zhang Z, Sun Z, Zhao J, Zhu D, Wang Y, et al. Vaccines Targeting PreS1 Domain Overcome Immune Tolerance in HBV Carrier Mice FIRS Public Access. 2017; 66:1067-1082.

Chen M, Jagya N, Bansal R, Frelin L. Sällberg M. Prospects and progress of DNA vaccines for treating hepatitis B. Expert Rev Vaccines 2016; 15:629-640.

Yalcin K, Danis R, Degertekin H, Alp M N, Tekes S, Budak T. The lack of effect of therapeutic vaccination with a pre-S2/S HBV vaccine in the immune tolerant phase of chronic HBV infection. J Clin Gastroenterol 2003; 37:330-5.

Zhao H-J, Han Q-J, Wang G, Lin A, Xu D-Q, Wang Y-Q, et al. I:C-based rHBVvac therapeutic vaccine eliminates HBV via generatioPolyn of HBV-specific CD8+effector memory T cells. Gut 2019; :gutjnl-2017-315588.

Suslov A, Boldanova T, Wang X, Wieland S, Heim M H Hepatitis B Virus Does Not Interfere With Innate Immune Responses in the Human Liver. Gastroenterology 2018; 154:1778-1790.

1. An immunogenic composition or product combination comprising:
(a) a nucleic acid comprising at least one nucleic acid sequence encoding hepatitis D antigen (HDAg) and at least one nucleic acid sequence encoding PreS1; and
(b) a polypeptide comprising at least one HDAg polypeptide sequence and at least one PreS1 polypeptide sequence.

2-49. (canceled)
